(19) United States
(12) Patent Application Publication
(10) Pub. No.: US 2020/0080045 A1
(43) Pub. Date: Mar. 12, 2020

(54) AUTOMATED CELL GROWTH AND/OR CONCENTRATION MODULES AS STAND-ALONE DEVICES OR FOR USE IN MULTI-MODULE CELL PROCESSING INSTRUMENTATION

(71) Applicant: Inscripta, Inc., Boulder, CO (US)

(72) Inventors: Jorge Bernate, Boulder, CO (US); Don Masquelier, Boulder, CO (US); Phillip Belgrader, Pleasanton, CA (US); Bruce Chabansky, Boulder, CO (US)

(21) Appl. No.: 16/561,701

(22) Filed: Sep. 5, 2019

Related U.S. Application Data
(60) Provisional application No. 62/728,365, filed on Sep. 7, 2018.
(60) Provisional application No. 62/857,599, filed on Jun. 5, 2019.
(60) Provisional application No. 62/867,415, filed on Jun. 27, 2019.

(51) Int. Cl. C12M 1/36; C12M 1/26; C12M 1/12; C12M 1/34

(52) U.S. Cl. CPC C12M 41/48; C12M 41/40; C12M 37/02; C12M 33/12

(57) ABSTRACT

The present disclosure provides a cell growth, buffer exchange, and/or cell concentration/filtration device that may be used as a stand-alone device or as a module configured to be used in an automated multi-module cell processing environment.

### RELATED APPLICATIONS

This application claims priority to U.S. Patent Application Ser. Nos. 62/728,365, filed Sep. 7, 2018; 62/857,599, filed Jun. 5, 2019; and 62/867,415, filed Jun. 27, 2019.

### FIELD OF THE INVENTION

The present disclosure provides a cell growth, buffer exchange, and/or cell concentration device that may be used as a stand-alone device or as a module configured to be used in an automated multi-module cell processing environment.

### BACKGROUND OF THE INVENTION

In the following discussion certain articles and methods will be described for background and introductory purposes. Nothing contained herein is to be construed as an “admission” of prior art. Applicant expressly reserves the right to demonstrate, where appropriate, that the articles and methods referenced herein do not constitute prior art under the applicable statutory provisions.

Genome editing with engineered nucleases is a method in which changes to nucleic acids are made in the genome of a living organism. Certain nucleases create site-specific double-strand breaks at target regions in the genome, which can be repaired by nonhomologous end-joining or homologous recombination resulting in targeted edits. Nucleases can be used to introduce one or more edits into multiple cells simultaneously, allowing for the production of libraries of cells with one or more edits in the cellular genome. These methods, however, generally have not been compatible with automation due to low transformation and editing efficiencies and challenges with cell growth and selection. In addition to genome editing, other multi-step cell processes would benefit from automation, including genome engineering, hybridoma production, and induction of protein synthesis.

In order to obtain an adequate number of cells for transformation or transfection, cells typically are grown to a specific optical density in milliliter or liter volumes in medium appropriate for the growth of the cells of interest; however, for effective transformation or transfection, it is desirable to decrease the volume of the cells as well as render the cells competent via buffer or medium exchange. Thus, one sub-component or module that is essential to cell processing systems for the processes listed above is a module or component that can grow, perform buffer exchange, and/or concentrate cells and render them competent so that they may be transformed or transfected with the nucleic acids needed for engineering or editing the cell's genome.

There is thus a need for automated stand-alone cell growth, buffer exchange, and/or concentration devices as well as cell growth and/or concentration modules that may be one module in a multi-module cell processing instruments where the cell growth and/or concentration modules are capable of growing, concentrating and rendering competent cells in an efficient and automated fashion. The present invention addresses this need.

### SUMMARY OF THE INVENTION

This Summary is provided to introduce a selection of concepts in a simplified form that are further described below in the Detailed Description. This Summary is not intended to identify key or essential features of the claimed subject matter, nor is it intended to be used to limit the scope of the claimed subject matter. Other features, details, utilities, and advantages of the claimed subject matter will be apparent from the following written Detailed Description including those aspects illustrated in the accompanying drawings and defined in the appended claims.

The present disclosure provides a cell growth and/or concentration device that not only grows and concentrates cells, but also in some aspects renders the cells being concentrated competent via medium/buffer exchange. The cell growth and/or concentration device may be used as a stand-alone device or as one module in a multi-module cell processing instrument. Also described are automated multi-module cell processing instruments including the cell growth and/or concentration devices or modules and methods of using the cell growth and/or concentration devices or modules. The cell growth and/or concentration device described herein operates using tangential flow filtration (TFF), also known as crossflow filtration), in which the majority of the feed flows tangentially over the surface of the filter. Tangential flow filtration reduces cake formation compared to dead-end filtration, in which the feed flows into the filter. Secondary flows relative to the main feed are also exploited to generate shear forces that prevent filter cake formation and membrane fouling thus maximizing particle recovery. The terms “cell growth, buffer exchange and/or concentration device”, “cell growth, buffer exchange, and/or concentration module”, “cell growth and/or concentration device”, “cell growth and/or concentration module”, “TFF device”, and “TFF module” are equivalent.

Thus, there is provided a tangential flow filtration (TFF) device comprising 1) a tangential flow assembly comprising: a retentate member comprising an upper surface and a lower surface with a retentate channel structure defining a flow channel disposed on the lower surface of the retentate member and first and second retentate ports wherein the first retentate port is disposed at a first end of the channel structure and the second retentate port is disposed at a second end of the channel structure, and wherein the first and second retentate ports traverse the first member from the lower surface to the upper surface; a permeate member comprising an upper surface and a lower surface with a permeate channel structure defining a flow channel disposed on the upper surface of the permeate member and at least one permeate port, wherein the at least one permeate port is disposed at a first end of the permeate channel structure, wherein the at least one permeate port traverses the permeate member from the lower surface to the upper surface, and wherein the channel structures of the retentate and permeate members mate to form a single flow channel; and a membrane disposed between the retentate and permeate members thereby bifurcating the single flow channel into upper and lower portions; 2) a reservoir assembly comprising a first retentate reservoir fluidically coupled to the first retentate port, a second retentate reservoir fluidically coupled to the second retentate port and a reservoir top disposed over the first and second retentate reservoirs; 3) a pneumatic assembly configured to apply pressure to move liquid through the single flow channel via negative and positive pressure applied to the first and second retentate reservoirs, to monitor pressure in the retentate reservoirs, and to monitor flow in the flow channel; 4) an interface between the pneumatic assembly and the reservoir top; and 5) means to couple the retentate member, the membrane, the permeate member, and the reservoir assembly.

In some aspects of this embodiment of a TFF, wherein the single flow channel has a serpentine configuration and in some aspects, the channel structure has an undulating geometry. In some aspects, the length of the single flow channel is from 100 mm to 500 mm, or from 150 mm to 400 mm, or from 200 mm to 350 mm.

In some embodiments, the reservoir assembly further comprises a first permeate reservoir fluidically coupled to the at least one permeate port. In some aspects, there is a second permeate port disposed at a second end of the permeate channel structure and the second permeate port also is fluidically coupled to the first permeate reservoir.

In some aspects, the reservoir assembly further comprises a buffer reservoir fluidically coupled to at least one of the first and second retentate reservoirs.

In some aspects, the cross section of the flow channel is rectangular or trapezoidal, and in some aspects, the cross section of the flow channel is 300 μm to 700 μm wide and 300 μm to 700 μm high. In yet other aspects, the cross section of the flow channel is generally circular, and the cross section of the flow channel is 300 μm to 700 μm in radius.

In some aspects, the reservoir assembly further comprises a gasket disposed on the reservoir top of the reservoir assembly and the gasket comprises a pneumatic port and a fluid transfer port for each of the first and second retentate reservoirs. In some aspects of this embodiment, the flow channel has a channel structure with a serpentine configuration that crisscrosses the retentate and permeate members, and in some aspects, the channel structure has other curved geometries. In yet other aspects, the TFF device has a serpentine configuration and an undulating geometry. In some aspects, the footprint length of the channel structure is from 10 mm to 1000 mm, from 60 mm to 200 mm, or from 80 mm to 100 mm. In some aspects, the entire footprint width of the channel structure is from 10 mm to 120 mm, from 40 mm to 70 mm, or from 50 mm to 60 mm.

In some aspects, the cross section of the flow channel is rectangular. In some aspects, the cross section of the flow channel is 5 μm to 1000 μm wide and 5 μm to 1000 μm high, 300 μm to 700 μm wide and 300 μm to 700 μm high, or 400 μm to 600 μm wide and 400 μm to 600 μm high. In other aspects, the cross section of the flow channel is circular, elliptical, trapezoidal, or oblong, and is 100 μm to 1000 μm in hydraulic radius, 300 μm to 700 μm in hydraulic radius, or 400 μm to 600 μm in hydraulic radius.

In some aspects, the means to couple or secure the retentate member, permeate member and membrane together is use of a pressure sensitive adhesive. In other aspects, the retentate member, permeate member and membrane are coupled or secured together by fasteners such as screws or clamps. In other aspects, the retentate member, permeate member and membrane are coupled or secured together by solvent bonding. In other aspects, the retentate member, permeate member and membrane are coupled or secured together by ultrasonic welding. In yet other aspects, the retentate member, permeate member and membrane are coupled or secured together by mated fittings.

Again, in some aspects, the channel structure has a serpentine configuration with local curved geometries that crisscrosses the retentate and permeate members; and in some aspects, the TFF device further comprises retentate reservoirs coupled to the retentate ports.

Also provided is an automated multi-module cell processing instrument comprising the tangential flow filtration device, and further comprising a transformation module and an automated liquid handling device configured to move liquids from the TFF device to the transformation module. In some aspects the automated multi-module cell processing system further comprises a reagent cartridge, and in some aspects, the reagent cartridge further comprises the transformation module. In some aspects, the transformation module is a flow-through electroporation device. In some aspects, there is also included in the automated multi-module cell processing instrument an isolation and editing module. In some aspects, the isolation and editing module is a solid wall isolation and editing module. In some aspects of the automated multi-module cell processing instrument, there is a growth module separate from the tangential flow filtration device.

Other embodiments provide method for growing a cell sample, comprising the steps of: providing one of the tangential flow filtration (TFF) devices described herein; providing a cell sample; placing the cell sample into the first retentate reservoir; passing the cell sample through the retentate channel structure for a length of the channel structure until the cell sample is transported into and retained within the second retentate reservoir; collecting filtrate through the permeate port; passing the cell sample from the second reservoir through the retentate channel structure for the length of the retentate channel structure until the cell sample is transported into and retained within the first reservoir; collecting filtrate through the permeate port; monitoring growth of the cell sample in the retentate reservoirs; repeating the passing, collecting, passing, collecting and monitoring steps until the cell sample has reached a desired stage of growth; and collecting the cell sample.

In some aspects, there is further provided the step of bubbling an appropriate gas through the cell culture while the cell culture is in one or both of the first and second reservoirs. In some aspects, growth of the cell sample is measured by optical density. In some aspects, medium is added to the cell sample in the first and/or second retentate reservoir to refresh the medium to enhance cell growth.

Also provided is a method for concentrating a cell sample, comprising the steps of providing tangential flow filtration (TFF) device; providing a cell sample in a first medium; placing the cell sample into the first retentate reservoir; passing the cell sample from the first retentate reservoir through the retentate channel structure for a length of the channel structure until the cell sample is transported into and retained within the second retentate reservoir; collecting filtrate through the permeate port; passing the cell sample from the second retentate reservoir through the retentate channel structure for the length of the channel structure until the cell sample is transported into and retained within the first retentate reservoir; collecting filtrate through the permeate port; and repeating the passing and collecting steps until the cell sample is concentrated to a desired volume.

In some aspects, this method further comprises the steps of adding a second medium to the cells in the first and/or second reservoirs where the second medium is different from the first medium, and repeating the passing and collecting steps until the cell sample is suspended in the second medium.

These aspects and other features and advantages of the invention are described below in more detail.

### BRIEF DESCRIPTION OF THE DRAWINGS

The foregoing and other features and advantages of the present invention will be more fully understood from the following detailed description of illustrative embodiments taken in conjunction with the accompanying drawings in which:

FIG. 1A is a model of tangential flow filtration used in the TFF module presented herein. FIG. 1B depicts a top view of the permeate member of one embodiment of an exemplary TFF device/module. FIG. 1C depicts a top view of retentate and permeate members and the membrane of an exemplary TFF module. FIG. 1D depicts a bottom view of retentate and permeate members of an exemplary TFF module. FIG. 1E depicts a side planar view of an exemplary assembled TFF module comprising retentate and permeate members, a filter, and retentate reservoirs. FIG. 1F depicts a top view of retentate and permeate members and the membrane of an exemplary TFF module with an alternative configuration of reservoirs as those shown in FIG. 1D. FIGS. 1G-1N depict various views of another embodiment of a TFF module having fluidically coupled reservoirs for retentate, filtrate, and exchange buffer. FIG. 1O depicts the circuitry of an exemplary TFF module such as that depicted in FIGS. 1G-1N. FIGS. 1P-1DD depict various views of three other embodiments of TFF modules with tangential flow members with fluidically coupled reservoirs: one embodiment comprising one permeate port and two retentate ports (FIGS. 1P, 1Q, 1BB and 1CC), and the other two embodiments comprising two permeate ports and two retentate ports (FIGS. 1R-1V and 1DD). FIGS. 1Y-1AA depict an exemplary top, with fluidic and pneumatic ports and gasket suitable for the reservoir assemblies described herein. FIG. 1EE is an exemplary pneumatic architecture diagram for the TFF modules described in relation to FIGS. 1R-1V and 1DD.

FIGS. 2A-2E depict various views of an exemplary automated multi-module cell processing instrument comprising a TFF device/module such as those depicted in FIGS. 1B-1EE.

FIG. 3A depicts an exemplary combination reagent cartridge and electroporation device that may be used in a multi-module cell processing instrument. FIG. 3B is a top perspective view of one embodiment of an exemplary flow-through electroporation device that may be part of a reagent cartridge. FIG. 3C depicts a bottom perspective view of one embodiment of an exemplary flow-through electroporation device that may be part of a reagent cartridge. FIGS. 3D-3M depict top perspective views, top views of a cross section, and side perspective view cross sections of various embodiments of FTEP devices described herein.

FIG. 4A depicts one embodiment of a rotating growth vial for use with the cell growth module described herein and in relation to FIGS. 4B-4D. FIG. 4B illustrates a perspective view of one embodiment of a rotating growth device in a cell growth module housing. FIG. 4C depicts a cut-away view of the cell growth module from FIG. 4B. FIG. 4D illustrates the cell growth module of FIG. 4B coupled to LED, detector, and temperature regulating components.

FIGS. 5A-5H depict a different embodiment of a SWIIN module, where the retentate and permeate members are coincident with reservoir assembly. FIG. 5I depicts the embodiment of the SWIIN module in FIGS. 5A-5H further comprising a heater and a heated cover. FIG. 5J is an exemplary pneumatic architecture diagram for the SWIIN module described in relation to FIGS. 5A-5H, with the status of the components for the various steps listed in Tables 4-6.

FIG. 6 is a block diagram of one embodiment of a method for using a TFF module as one module in an automated multi-module cell processing instrument.

FIG. 7 is a simplified process diagram of an exemplary automated multi-module cell processing instrument in which one or more of the TFF modules described herein may be used.

FIG. 8 is a simplified process diagram of a different embodiment of an exemplary automated multi-module cell processing instrument in which one or more of the TFF modules described herein may be used.

FIG. 9 is a simplified process diagram of yet another embodiment of an exemplary automated multi-module cell processing instruments in which one or more of the TFF modules described herein may be used.

FIG. 10A shows plots of cell optical density vs. time for E. coli cell cultures grown in a traditional shaker and in a TFF device. FIG. 10B shows plots of cell optical density vs. time for yeast cell cultures grown in a traditional shaker and in a TFF device.

FIG. 11A is a graph plotting filtrate conductivity against filter processing time for an E. coli culture processed in the cell growth and/or concentration device/module described herein. FIG. 11B is a graph plotting filtrate conductivity against filter processing time for a yeast culture processed in the cell growth and/or concentration device/module described herein.

It should be understood that the drawings are not necessarily to scale, and that like reference numbers refer to like features.

### DETAILED DESCRIPTION

All of the functionalities described in connection with one embodiment are intended to be applicable to the additional embodiments described herein except where expressly stated or where the feature or function is incompatible with the additional embodiments. For example, where a given feature or function is expressly described in connection with one embodiment but not expressly mentioned in connection with an alternative embodiment, it should be understood that the feature or function may be deployed, utilized, or implemented in connection with the alternative embodiment unless the feature or function is incompatible with the alternative embodiment.

The practice of the techniques described herein may employ, unless otherwise indicated, conventional techniques and descriptions of molecular biology (including recombinant techniques), cell biology, biochemistry, and genetic engineering technology, which are within the skill of those who practice in the art. Such conventional techniques and descriptions can be found in standard laboratory manuals such as Green and Sambrook, Molecular Cloning: A Laboratory Manual. 4th, ed., Cold Spring Harbor Laboratory Press, Cold Spring Harbor, N.Y., (2014); Current Protocols in Molecular Biology, Ausubel, et al. eds., (2017); Cell and Tissue Culture: Laboratory Procedures in Biotechnology (Doyle & Griffiths, eds., John Wiley & Sons 1998); Mammalian Chromosome Engineering—Methods and Protocols (G. Hadlaczky, ed., Humana Press 2011); Essential Stem Cell Methods, (Lanza and Klimanskaya, eds., Academic Press 2011); Neumann, et al., Electroporation and Electrofusion in Cell Biology, Plenum Press, New York, 1989; and Chang, et al., Guide to Electroporation and Electrofusion, Academic Press, California (1992), all of which are herein incorporated in their entirety by reference for all purposes. CRISPR-specific techniques can be found in, e.g., Genome Editing and Engineering from TALENs and CRISPRs to Molecular Surgery, Appasani and Church (2018); and CRISPR: Methods and Protocols, Lindgren and Charpentier (2015); both of which are herein incorporated in their entirety by reference for all purposes.

Note that as used herein and in the appended claims, the singular forms “a,” “an,” and “the” include plural referents unless the context clearly dictates otherwise. Thus, for example, reference to “a cell” refers to one or more cells, and reference to “the instrument” includes reference to equivalent steps, methods and devices known to those skilled in the art, and so forth.

Unless defined otherwise, all technical and scientific terms used herein have the same meaning as commonly understood by one of ordinary skill in the art to which this invention belongs. All publications mentioned herein are incorporated by reference for the purpose of describing and disclosing devices, formulations and methodologies that may be used in connection with the presently described invention.

Where a range of values is provided, it is understood that each intervening value, between the upper and lower limit of that range and any other stated or intervening value in that stated range is encompassed within the invention. The upper and lower limits of these smaller ranges may independently be included in smaller ranges, and are also encompassed within the invention, subject to any specifically excluded limit in the stated range. Where the stated range includes one or both of the limits, ranges excluding either or both of those included limits are also included in the invention.

In the following description, numerous specific details are set forth to provide a more thorough understanding of the present invention. However, it will be apparent to one of skill in the art that the present invention may be practiced without one or more of these specific details. In other instances, features and procedures well known to those skilled in the art have not been described in order to avoid obscuring the invention. The terms used herein are intended to have the plain and ordinary meaning as understood by those of ordinary skill in the art.

### The Invention in General

The present disclosure relates to a cell growth, concentration and medium exchange device/module for growing and concentrating cells and, in some embodiments, rendering cells competent. The cell growth and/or concentration device/module (e.g., tangential flow filtration module or TFF module) can be used as a stand-alone device or as one module as part of an automated multi-module cell processing instrument. The automated multi-module cell processing instrument can be used to process many different types of cells in a controlled, contained, and reproducible manner, including bacterial cells, yeast cells, mammalian cells, other non-mammalian eukaryotic cells, plant cells, fungi, and the like. The cell processes that may be performed include genome engineering, cell transformation, cell culture and/or selection, genome editing and recursive editing, protein production and production of hybridomas.

### The Cell Growth, Buffer Exchange, and Concentration TFF Device or Module

The present disclosure provides a cell growth, buffer exchange, and/or concentration device (module) that not only grows and concentrates cells, but also in some aspects renders the cells being concentrated competent via medium/buffer exchange. The tangential flow filtration device or TFF device may be used as a stand-alone device or, in some embodiments, as one module in a multi-module cell processing instrument. Also described are automated multi-module cell processing instruments and systems including the TFF devices or modules and methods of using the TFF devices or modules. The TFF cell growth and/or concentration device described herein operates using tangential flow filtration (TFF), also known as crossflow filtration, in which the majority of the feed flows tangentially over or across the surface of the filter thereby reducing cake (retentate) formation as compared to dead-end filtration, in which the feed flows into the filter. Secondary flows relative to the main feed are also exploited to generate shear forces that prevent filter cake formation and membrane fouling thus maximizing particle recovery, as described below.

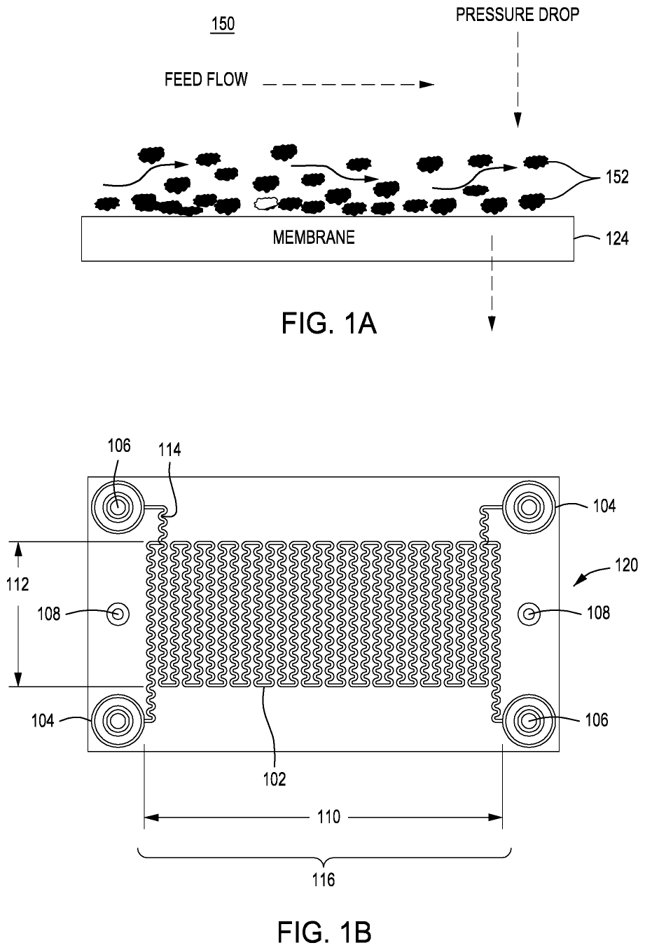

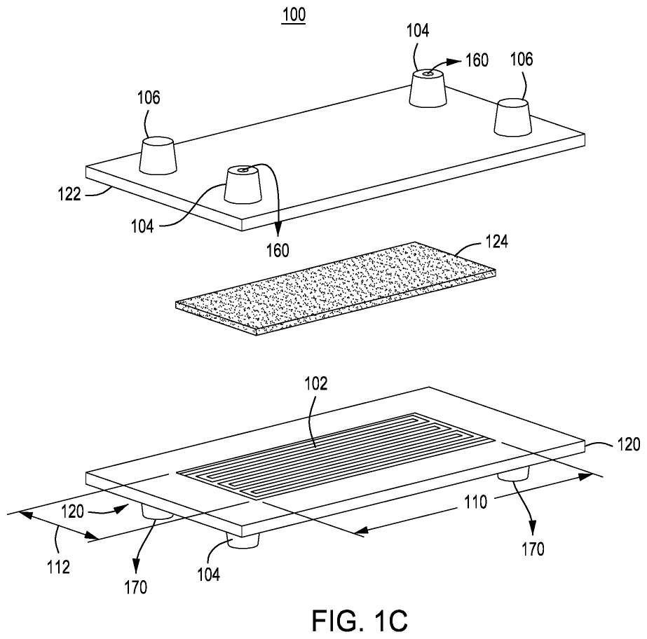

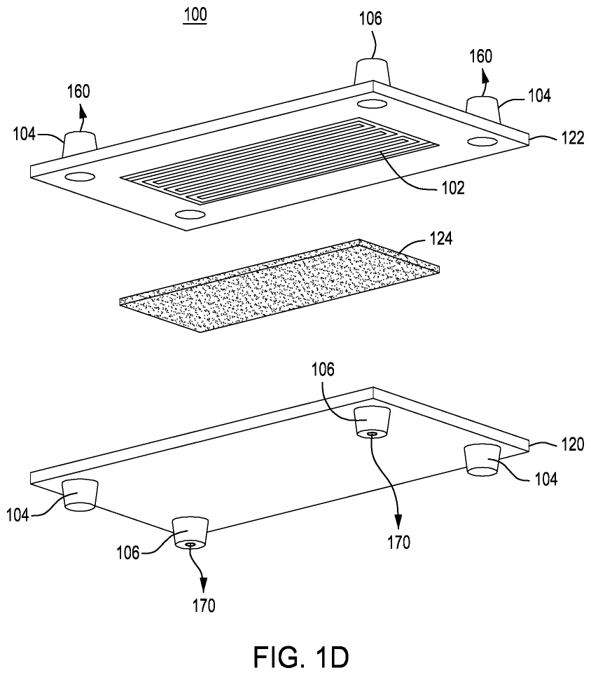

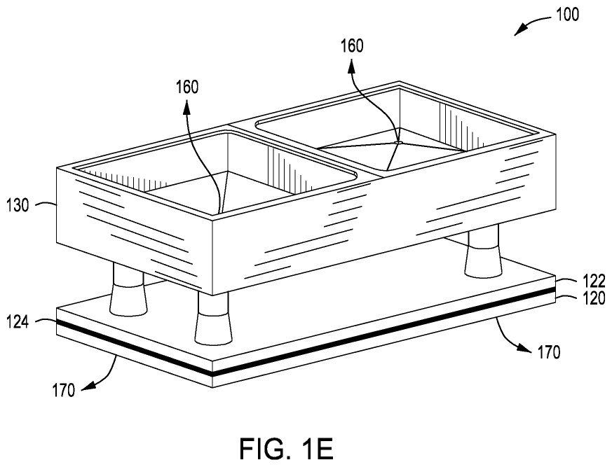

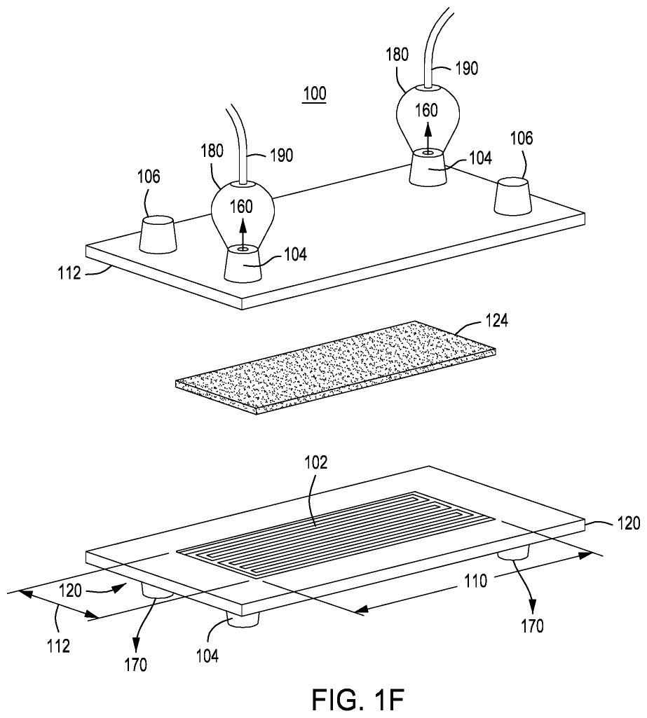

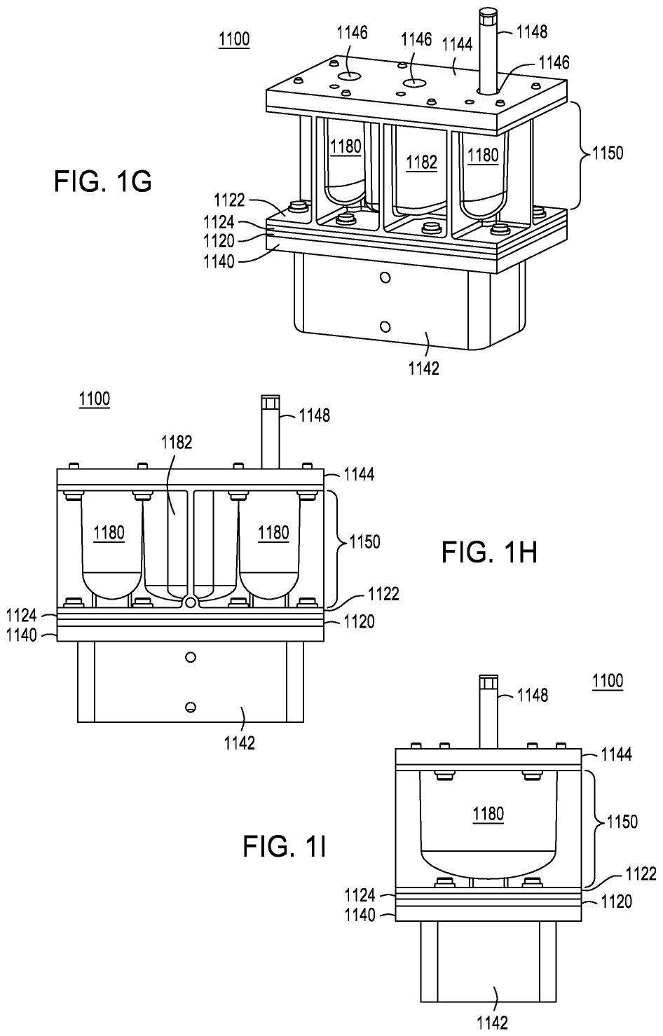

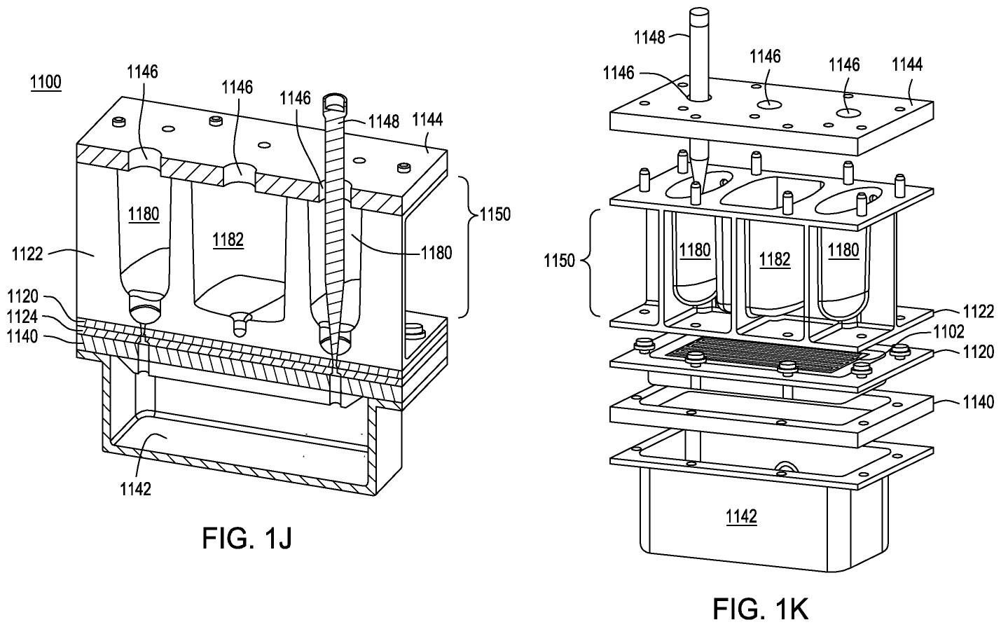

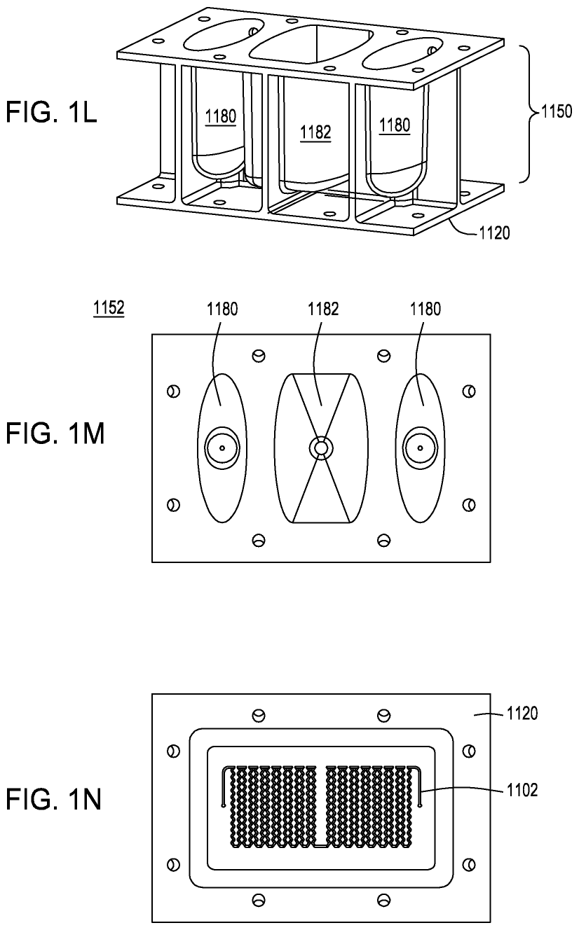

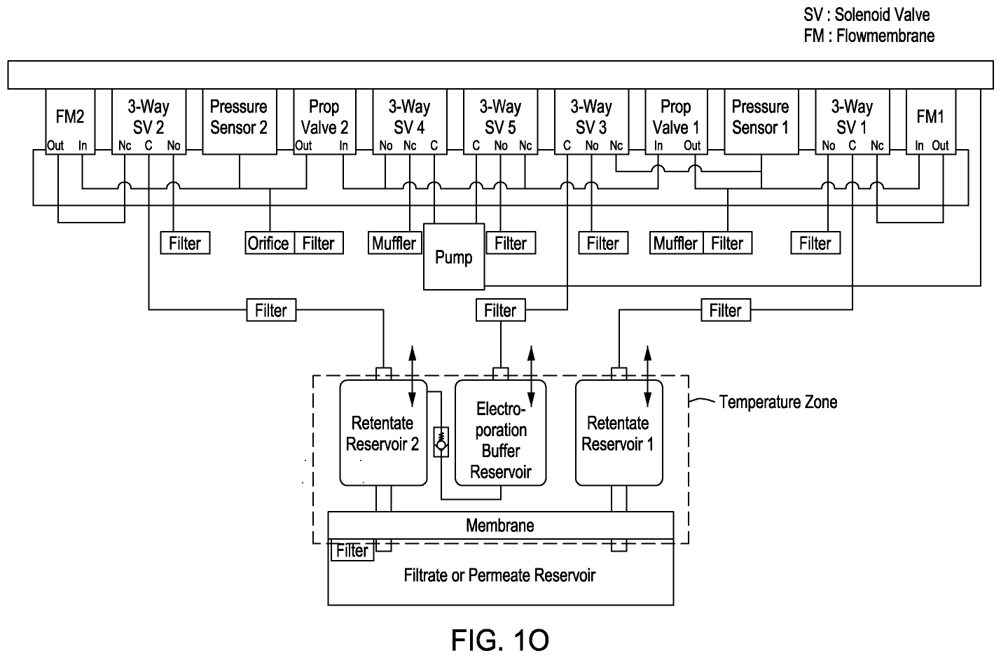

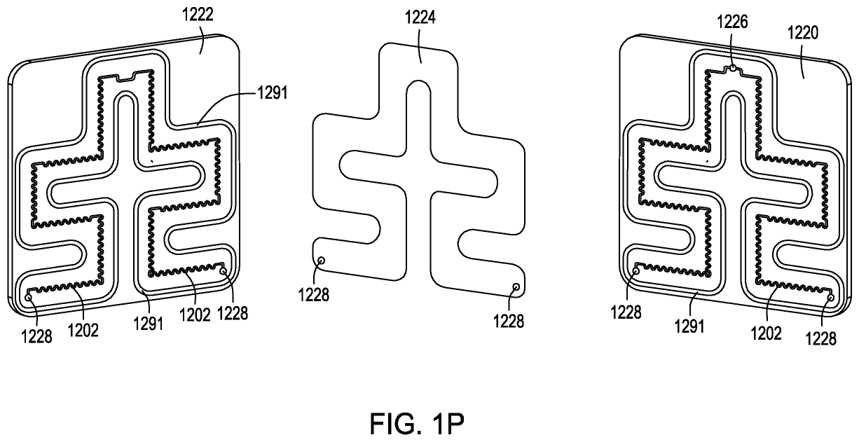

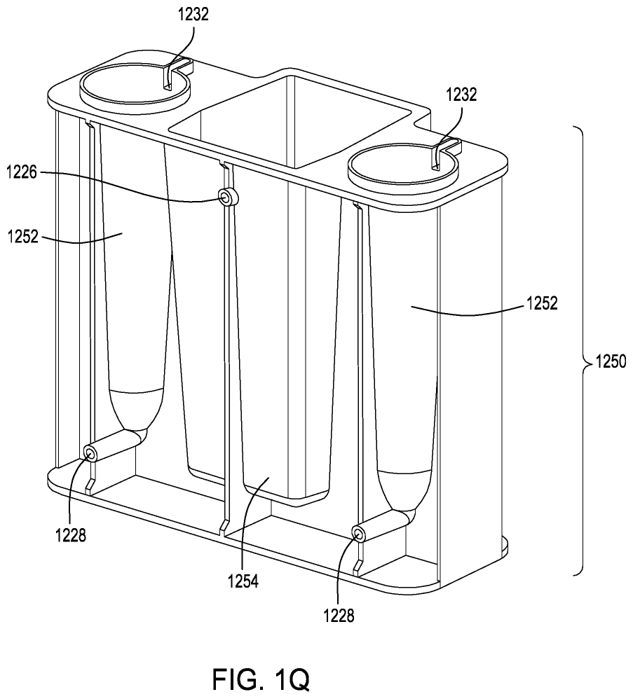

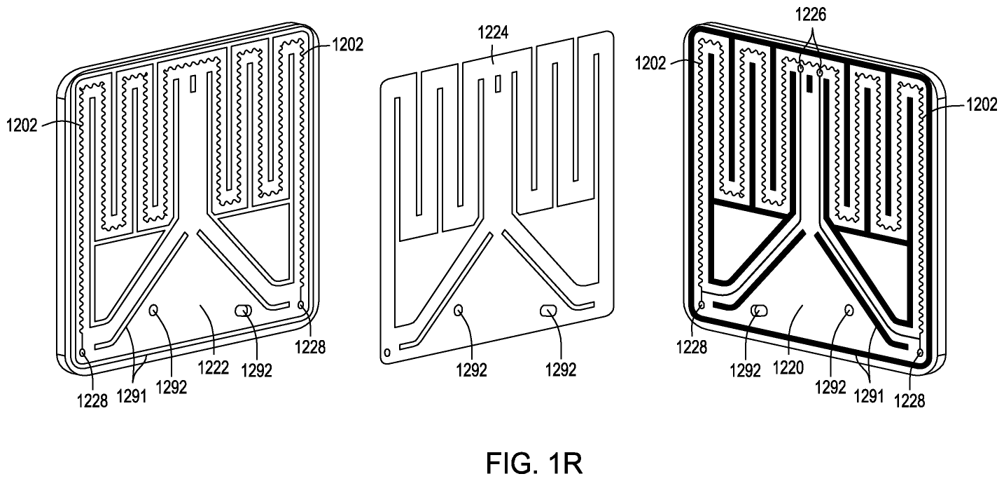

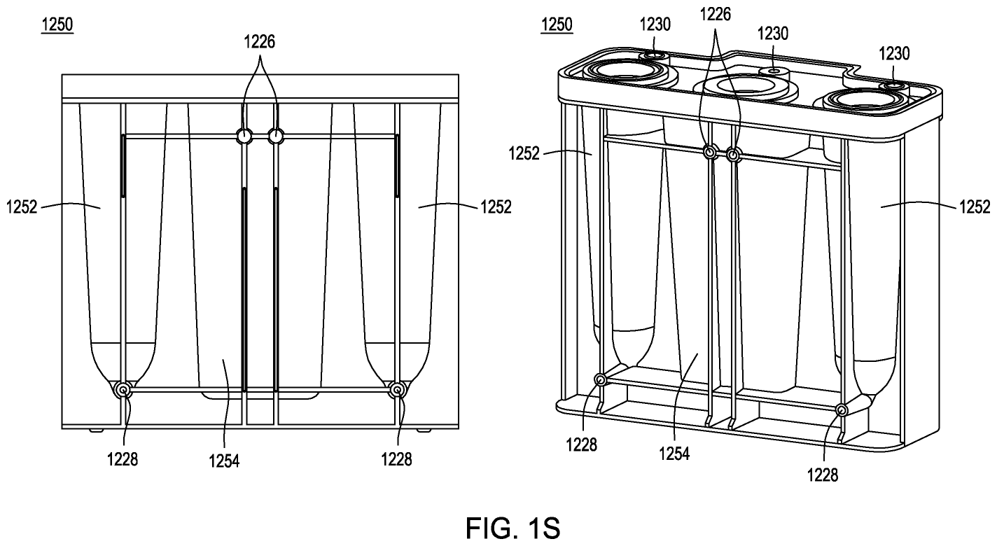

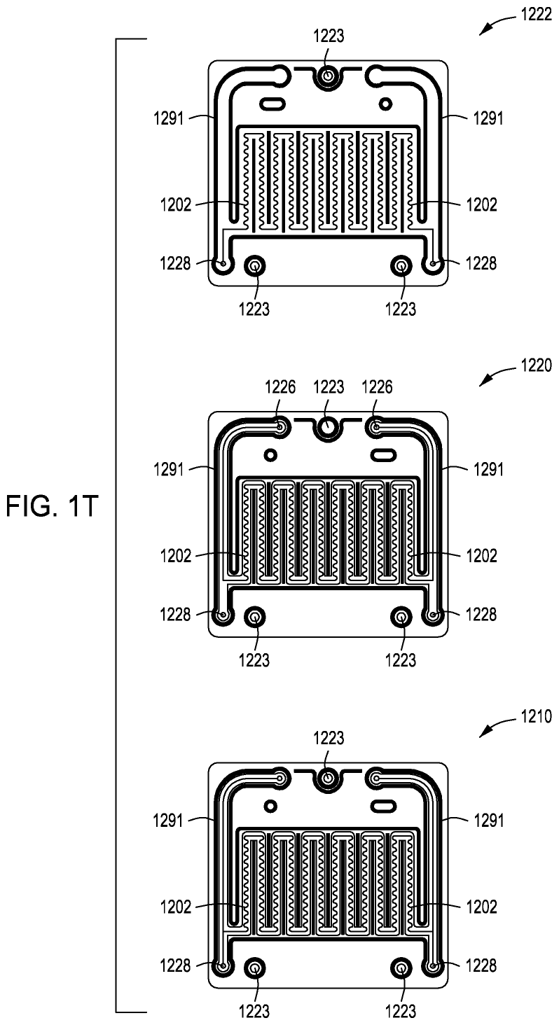

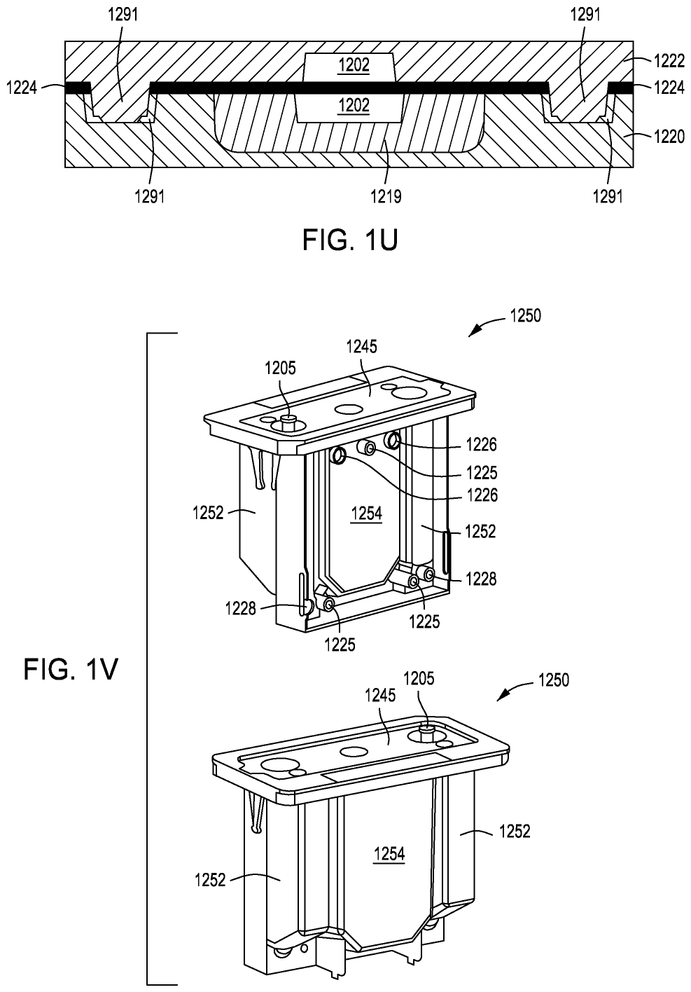

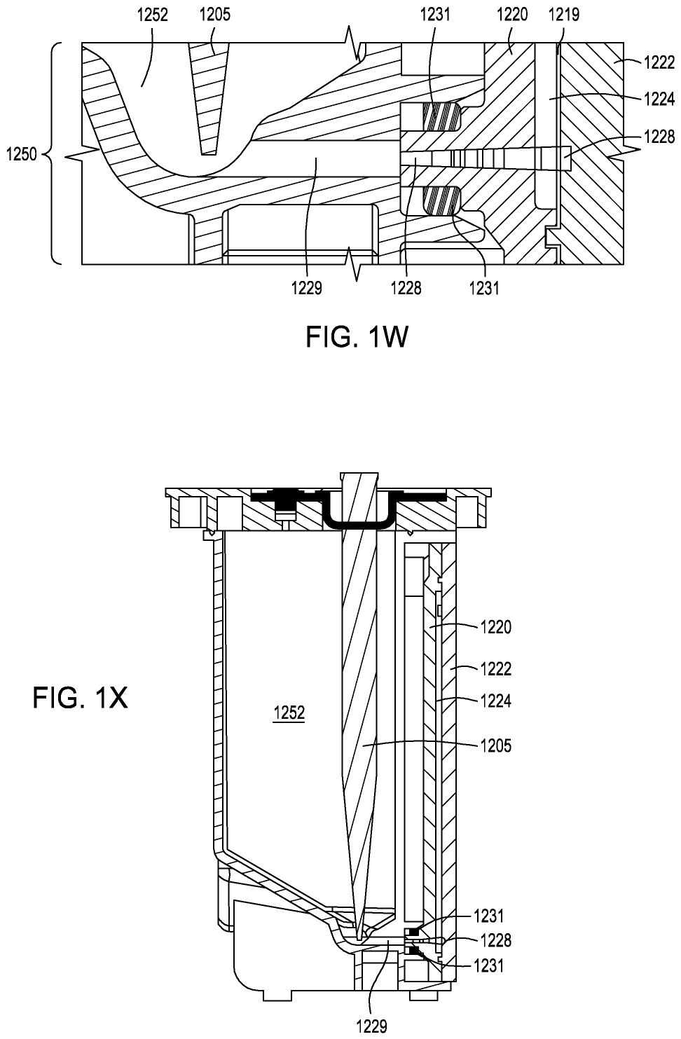

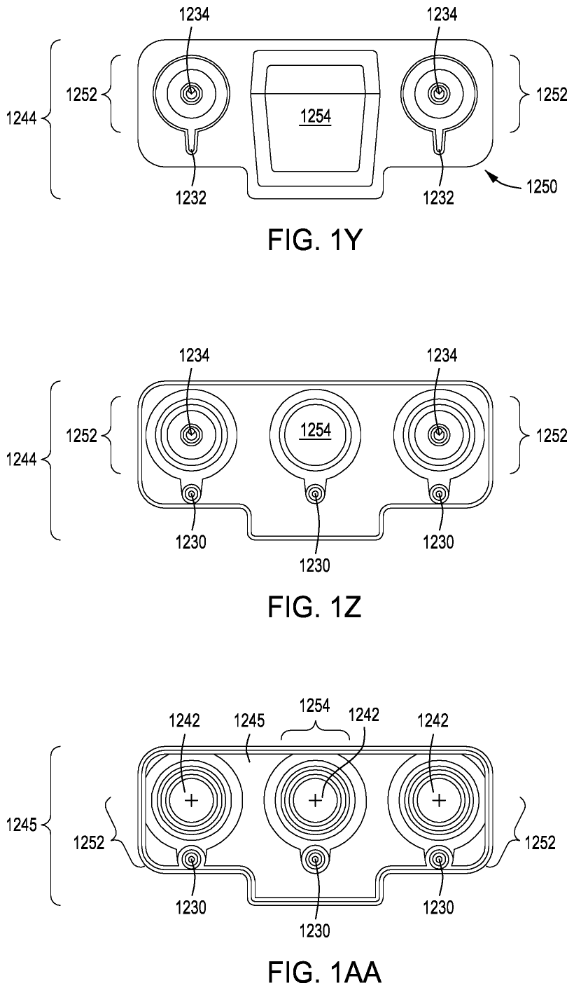

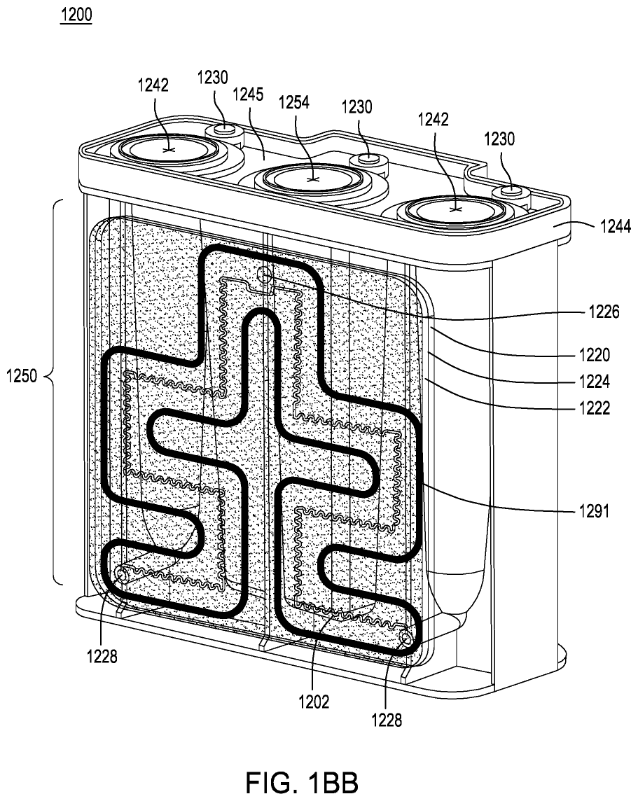

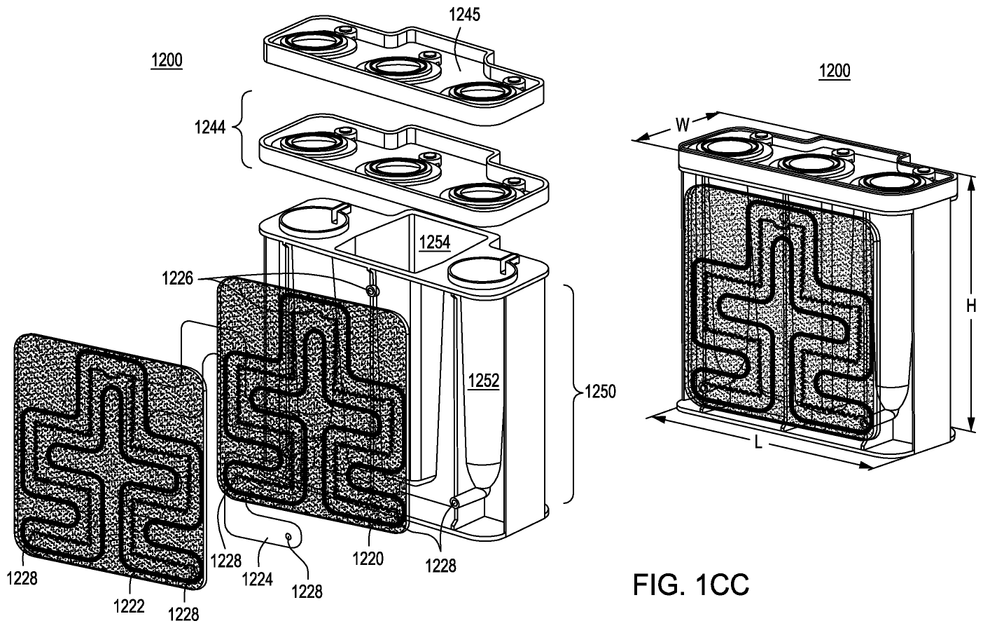

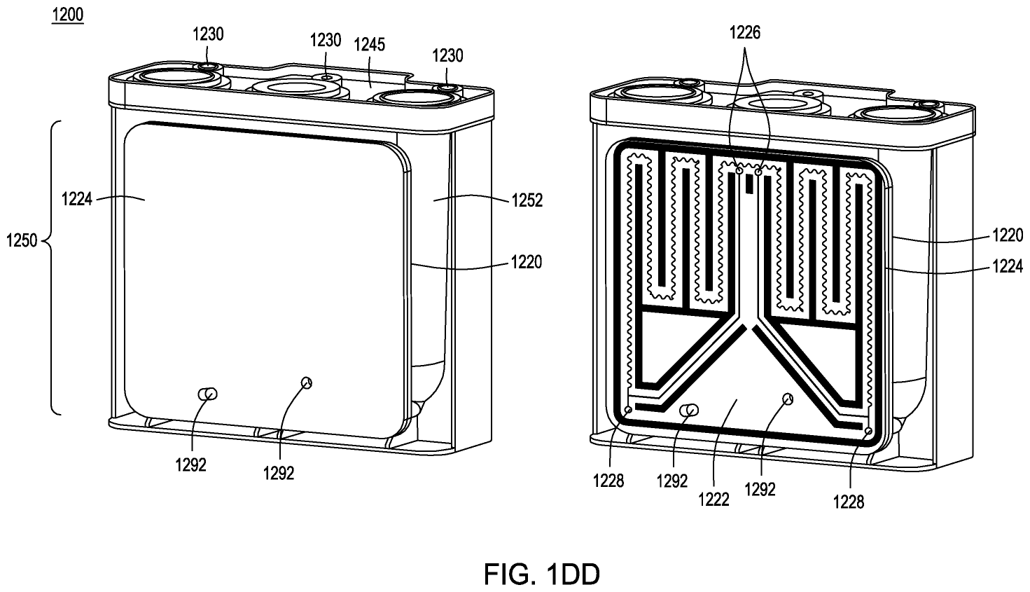

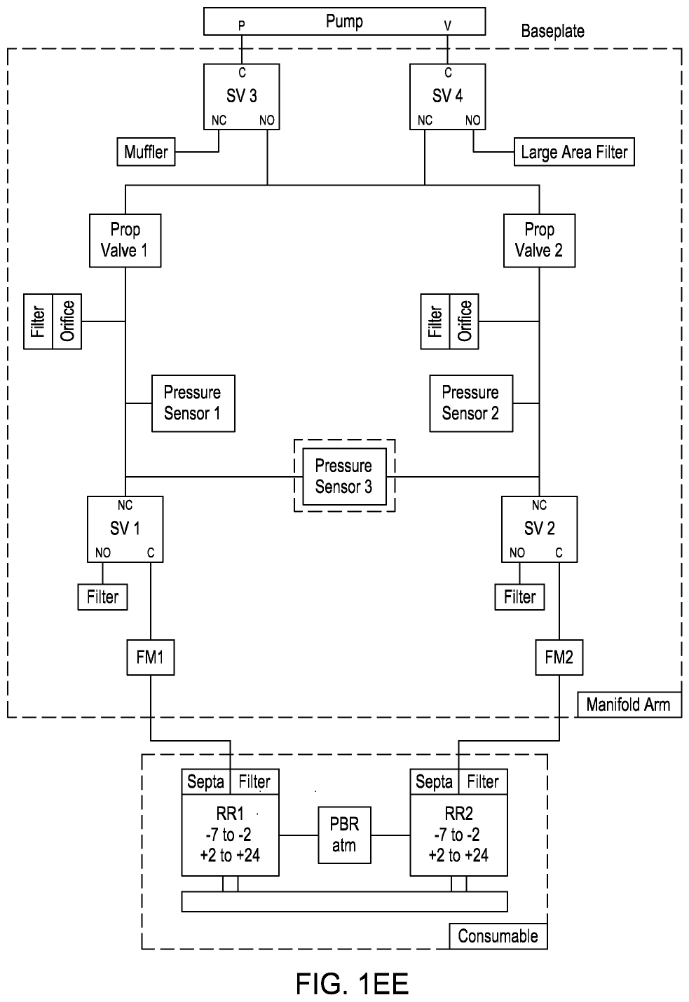

The TFF device described herein was designed to take into account two primary design considerations. First, the geometry of the TFF device leads to filtration of a cell culture over a large surface area so as to minimize processing time. Second, the design of the TFF device is configured to minimize filter fouling. FIG. 1A is a general model 150 of tangential flow filtration. FIG. 1A shows cells flowing over (rather than directly through) a membrane 124, where the feed flow of the cells 152 in medium or buffer is parallel to the membrane 124. TFF is different from dead-end filtration where both the feed flow and the pressure drop are perpendicular to a membrane or filter.

FIG. 1B depicts a top view of one embodiment of the permeate member 120 of a TFF device/module providing tangential flow filtration. As can be seen in the embodiment of the TFF device of FIG. 1B, TFF permeate member 120 comprises a channel structure 116 comprising a flow channel 102 through which a cell culture is flowed. The channel structure 116 comprises a single flow channel 102 that is horizontally bifurcated by a membrane (not shown) through which buffer or medium may flow, but cells cannot. This particular embodiment comprises an undulating serpentine geometry 114 (i.e., the small “wiggles” in the flow channel 102) and a serpentine “zig-zag” pattern where the flow channel 102 crisscrosses the device from one end at the left of the device to the other end at the right of the device. The serpentine pattern allows for filtration over a high surface area relative to the device size and total channel volume, while the undulating contribution creates a secondary inertial flow to enable effective membrane regeneration preventing membrane fouling. Although an undulating geometry and serpentine pattern are exemplified here, other channel configurations may be used as long as the channel can be bifurcated by a membrane and as long as the channel configuration provides for flow through the TFF module in alternating directions. In addition to the flow channel 102, ports 104 and 106 as part of the channel structure 116 can be seen, as well as recesses 108. Ports 104 collect cells passing through the channel on one side of a membrane (not shown) (e.g., the “retentate” side of the membrane), and ports 106 collect the medium (“filtrate” or “permeate”) passing through the channel on the opposite side of the membrane (not shown) (e.g., the “permeate” side of the membrane). In this embodiment, recesses 108 accommodate screws or other fasteners (not shown) that allow the components of the TFF device to be secured to one another.

The length 110 and width 112 of the channel structure 116 may vary depending on the volume of the cell culture to be grown and the optical density of the cell culture to be concentrated. The length 110 of the channel structure 116 typically is from 1 mm to 300 mm, or from 50 mm to 250 mm, or from 60 mm to 200 mm, or from 70 mm to 150 mm, or from 80 mm to 100 mm. The width of the channel structure 116 typically is from 1 mm to 120 mm, or from 20 mm to 100 mm, or from 30 mm to 80 mm, or from 40 mm to 70 mm, or from 50 mm to 60 mm. The cross-section configuration of the flow channel 102 may be round, elliptical, oval, square, rectangular, trapezoidal, or irregular. If square, rectangular, or another shape with generally straight sides, the cross section may be from about 10 μm to 1000 μm wide, or from 200 μm to 800 μm wide, or from 300 μm to 700 μm wide, or from 400 μm to 600 μm wide; and from about 10 μm to 1000 μm high, or from 200 μm to 800 μm high, or from 300 μm to 700 μm high, or from 400 μm to 600 μm high. If the cross section of the flow channel 102 is generally round, oval or elliptical, the radius of the channel may be from about 50 μm to 1000 μm in hydraulic radius, or from 5 μm to 800 μm in hydraulic radius, or from 200 μm to 700 μm in hydraulic radius, or from 300 μm to 600 μm wide in hydraulic radius, or from about 200 to 500 μm in hydraulic radius. Moreover, the volume of the channel in the retentate 122 and permeate 120 members may be different depending on the depth of the channel in each member.

When looking at the top view of the TFF device/module of FIG. 1B, note that there are two retentate ports 104 and two permeate/filtrate ports 106, where there is one of each type port at both ends (e.g., the narrow edge) of permeate member 120. In other embodiments, retentate and permeate/filtrate ports may be configured differently. Unlike other TFF devices that operate continuously—e.g., flowing cells from an entry port to an exit port—the TFF device/module described herein uses an alternating method for passing cells through the TFF and for concentrating cells.

The overall work flow for cell growth comprises loading a cell culture to be grown into a first retentate reservoir (not shown), optionally bubbling air or an appropriate gas through the cell culture, passing or flowing the cell culture through the first retentate port then tangentially through the TFF channel structure while collecting medium or buffer through one or both of the permeate ports 106, collecting the cell culture through a second retentate port 104 into a second retentate reservoir (not shown), optionally adding additional or different medium to the cell culture and optionally bubbling air or gas through the cell culture, then repeating the process, all while measuring, e.g., the optical density of the cell culture in the retentate reservoirs continuously or at desired intervals. Measurements of optical densities (OD) at programmed time intervals are accomplished using a 600 nm Light Emitting Diode (LED) that has been columnated through an optic into the retentate reservoir(s) containing the growing cells. The light continues through a collection optic to the detection system which consists of a (digital) gain-controlled silicone photodiode. Generally, optical density is shown as the absolute value of the logarithm with base 10 of the power transmission factors of an optical attenuator: OD=−log 10 (Power out/Power in). Since OD is the measure of optical attenuation—that is, the sum of absorption, scattering, and reflection—the TFF device OD measurement records the overall power transmission, so as the cells grow and become denser in population, the OD (the loss of signal) increases. The OD system is pre-calibrated against OD standards with these values stored in an on-board memory accessible by the measurement program.

In the channel structure, the membrane bifurcating the flow channels retains the cells on one side of the membrane (the retentate side) and allows unwanted medium or buffer to flow across the membrane into a filtrate or permeate side (e.g., permeate member 120) of the device. Bubbling air or other appropriate gas through the cell culture both aerates and mixes the culture to enhance cell growth. During the process, medium that is removed during the flow through the channel structure is removed through the permeate/filtrate ports 106. Alternatively, cells can be grown in one reservoir with bubbling or agitation without passing the cells through the TFF channel from one reservoir to the other.

The overall work flow for cell concentration using the TFF device/module involves flowing a cell culture or cell sample tangentially through the channel structure. As with the cell growth process, the membrane bifurcating the flow channels retains the cells on one side of the membrane and allows unwanted medium or buffer to flow across the membrane into a permeate/filtrate side (e.g., permeate member 120) of the device. In this process, a fixed volume of cells in medium or buffer is driven through the device until the cell sample is collected into one of the retentate ports 104, and the medium/buffer that has passed through the membrane is collected through one or both of the permeate/filtrate ports 106. All types of prokaryotic and eukaryotic cells—both adherent and non-adherent cells—can be grown in the TFF device. Adherent cells may be grown on beads or other cell scaffolds suspended in medium that flow through the TFF device.

The medium or buffer used to suspend the cells in the cell concentration device/module may be any suitable medium or buffer for the type of cells being transformed or transfected, such as SOC, MEM, DMEM, IMDM, RPMI, Hanks', PBS and Ringer's solution, where the media may be provided in a reagent cartridge as part of a kit. For culture of adherent cells, cells may be disposed on beads, microcarriers, or other type of scaffold suspended in medium. Most normal mammalian tissue-derived cells except those derived from the hematopoietic system—are anchorage dependent and need a surface or cell culture support for normal proliferation. In the rotating growth vial described herein, microcarrier technology is leveraged. Microcarriers of particular use typically have a diameter of 100-300 μm and have a density slightly greater than that of the culture medium (thus facilitating an easy separation of cells and medium for, e.g., medium exchange) yet the density must also be sufficiently low to allow complete suspension of the carriers at a minimum stirring rate in order to avoid hydrodynamic damage to the cells. Many different types of microcarriers are available, and different microcarriers are optimized for different types of cells. There are positively charged carriers, such as Cytodex 1 (dextran-based, GE Healthcare), DE-52 (cellulose-based, Sigma-Aldrich Labware), DE-53 (cellulose-based, Sigma-Aldrich Labware), and HLX 11-170 (polystyrene-based); collagen- or ECM- (extracellular matrix) coated carriers, such as Cytodex 3 (dextran-based, GE Healthcare) or HyQ-sphere Pro-F 102-4 (polystyrene-based, Thermo Scientific); non-charged carriers, like HyQ-sphere P 102-4 (Thermo Scientific); or macroporous carriers based on gelatin (Cultisphere, Percell Biolytica) or cellulose (Cytopore, GE Healthcare).

In both the cell growth and concentration processes, passing the cell sample through the TFF device and collecting the cells in one of the retentate ports 104 while collecting the medium in one of the permeate/filtrate ports 106 is considered “one pass” of the cell sample. The transfer between retentate reservoirs “flips” the culture. The retentate and permeate ports collecting the cells and medium, respectively, for a given pass reside on the same end of TFF device/module 100 with fluidic connections arranged so that there are two distinct flow layers for the retentate and permeate/filtrate sides, but if the retentate port 104 resides on the retentate member of device/module 100 (that is, the cells are driven through the channel above the membrane and the filtrate (medium) passes to the portion of the channel below the membrane), the permeate/filtrate port 106 will reside on the permeate member of device/module 100 and vice versa (that is, if the cell sample is driven through the channel below the membrane, the filtrate (medium) passes to the portion of the channel above the membrane). Due to the high pressures used to transfer the cell culture and fluids through the flow channel of the TFF device, the effect of gravity is negligible. The TFF device or module can be seen more clearly in FIGS. 1C-1F, where the retentate flows 160 from the retentate ports 104 and the filtrate flows 170 from the permeate/filtrate ports 106.

At the conclusion of a “pass” in either of the growth and concentration processes, the cell sample is collected by passing through the retentate port 104 and into the retentate reservoir (not shown). To initiate another “pass”, the cell sample is passed again through the TFF device, this time in a flow direction that is reversed from the first pass. The cell sample is collected by passing through the retentate port 104 and into retentate reservoir (not shown) on the opposite end of the device/module from the retentate port 104 that was used to collect cells during the first pass. Likewise, the medium/buffer that passes through the membrane on the second pass is collected through the permeate port 106 on the opposite end of the device/module from the permeate port 106 that was used to collect the filtrate during the first pass, or through both ports. Alternatively, there may be a single permeate reservoir configured to collect the permeate fluid as is the case with the TFF embodiment depicted in FIGS. 1P and 1Q. This alternating process of passing the retentate (the concentrated cell sample) through the device/module is repeated until the cells have been grown to a desired optical density, and/or concentrated to a desired volume, and both permeate ports (i.e., if there are more than one) can be open during the passes to reduce operating time. In addition, buffer exchange may be effected by adding a desired buffer (or fresh medium) to the cell sample in the retentate reservoir, before initiating another “pass”, and repeating this process until the old medium or buffer is diluted and filtered out and the cells reside in fresh medium or buffer. Note that buffer exchange and cell growth may (and typically do) take place simultaneously, and buffer exchange and cell concentration may (and typically do) take place simultaneously.

FIG. 1C depicts a top view of retentate (122) and permeate (120) members of an exemplary TFF module. Again, ports 104 and 106 are seen. As noted above, recesses—such as the recesses 108 seen in FIG. 1B—provide a means to secure the components (retentate member 122, permeate member 120, and membrane 124) of the TFF device/membrane to one another during operation via, e.g., screws or other like fasteners. However, in alternative embodiments an adhesive, such as a pressure sensitive adhesive, or ultrasonic welding, or solvent bonding may be used to couple the retentate member 122, permeate member 120, and membrane 124 together. Indeed, one of ordinary skill in the art given the guidance of the present disclosure can find yet other configurations for coupling the components of the TFF device, such as e.g., clamps; mated fittings disposed on the retentate and permeate members; combinations of adhesives, welding, solvent bonding, and mated fittings; and other such fasteners and couplings.

Note that there is one retentate port and one permeate port on each “end” (e.g., the narrow edges) of the TFF device/module. The retentate and permeate ports on the left side of the device/module will collect cells (flow path at 160) and medium (flow path at 170), respectively, for the same pass. Likewise, the retentate and permeate ports on the right side of the device/module will collect cells (flow path at 160) and medium (flow path at 170), respectively, for the same pass. In this embodiment, the retentate is collected from ports 104 on the top surface of the TFF device, and filtrate is collected from ports 106 on the bottom surface of the device. The cells are maintained in the TFF flow channel above the membrane 124, while the filtrate (medium) flows through membrane 124 and then through ports 106; thus, the top/retentate ports and bottom/filtrate ports configuration is practical. It should be recognized, however, that other configurations of retentate and permeate ports may be implemented such as positioning both the retentate and permeate ports on the side (as opposed to the top and bottom surfaces) of the TFF device.

In FIG. 1C, the channel structure 102 can be seen on the bottom member 120 of the TFF device 100. Also seen in FIG. 1C is membrane or filter 124. Filters or membranes appropriate for use in the TFF device/module are those that are solvent resistant, are contamination free during filtration, and are able to retain the types and sizes of cells of interest. For example, in order to retain small cell types such as bacterial cells, pore sizes can be as low as 0.2 μm, however for other cell types, the pore sizes can be as high as 20 μm or more. Indeed, the pore sizes useful in the TFF device/module include filters with sizes from 0.20 μm, 0.21 μm, 0.22 μm, 0.23 μm, 0.24 μm, 0.25 μm, 0.26 μm, 0.27 μm, 0.28 μm, 0.29 μm, 0.30 μm, 0.31 μm, 0.32 μm, 0.33 μm, 0.34 μm, 0.35 μm, 0.36 μm, 0.37 μm, 0.38 μm, 0.39 μm, 0.40 μm, 0.41 μm, 0.42 μm, 0.43 μm, 0.44 μm, 0.45 μm, 0.46 μm, 0.47 μm, 0.48 μm, 0.49 μm, 0.50 μm, 1.0 μm, 5.0 μm, 10.0 μm. 20.0 μm, 25.0 μm, 30.o μm, 40.0 μm, 50.0 μm and larger. The filters may be fabricated from any suitable non-reactive material including cellulose mixed ester (cellulose nitrate and acetate) (CME), polycarbonate (PC), polyvinylidene fluoride (PVDF), polyethersulfone (PES), polytetrafluoroethylene (PTFE), nylon, glass fiber, or metal substrates as in the case of laser or electrochemical etching. The TFF device shown in FIGS. 1C, 1D, and 1F do not show a seat in the retentate 112 and permeate 120 members where the filter 124 can be seated or secured (for example, a seat half the thickness of the filter in each of retentate 112 and permeate 120 members); however, such a seat is contemplated in some embodiments.

FIG. 1D depicts a bottom view of retentate and permeate members of the exemplary TFF module shown in FIG. 1C. FIG. 1D depicts a bottom view of retentate (122) and permeate (120) components of an exemplary TFF module. Again ports 104 and 106 are seen. Note again that there is one retentate port and one permeate/filtrate port on each end of the device/module. The retentate and permeate ports on the left side of the device/module will collect cells (flow path at 160) and medium (flow path at 170), respectively, for the same pass. Likewise, the retentate and permeate ports on the right side of the device/module will collect cells (flow path at 160) and medium (flow path at 170), respectively, for the same pass. In FIG. 1D, the channel structure 102 can be seen on the retentate member 122 of the TFF device 100. Thus, looking at FIGS. 1C and 1D, note that there is a channel structure 102 in both the retentate and permeate members, with a membrane 124 between the upper and lower portions of the channel structure. The channel structure 102 of the retentate 122 and permeate 120 members mate to create the flow channel with the membrane 124 positioned horizontally between the retentate and permeate members of the flow channel thereby bifurcating the flow channel.

FIG. 1E depicts a side planar view of an exemplary assembled TFF module comprising retentate and permeate members (122 and 120, respectively), a filter or membrane 124 sandwiched between the retentate 122 and permeate 120 members, permeate/filtrate ports, and retentate ports where the retentate ports are coupled to retentate reservoir 130. The flow path of the cells (retentate) is shown at 160. Retentate reservoir 130 collects the cells at each pass of the cells though the TFF device/module 100, whether during the growth phase (and/or buffer exchange) of the cell culture or during the concentration/buffer exchange phase of the cell culture. Note that buffer exchange and cell growth may (and typically do) take place simultaneously, and buffer exchange and cell concentration may (and typically do) take place simultaneously. The permeate/filtrate ports 106 are on the bottom surface of the permeate member 120 of the device/module 100; and the filtrate flow is shown at 170. Because the filtrate (medium/buffer) most typically comprises waste, it is not necessarily collected. Instead, the filtrate can be carried away from the TFF device/module 100 by, e.g., tubing (not shown), to a waste reservoir (also not shown).

FIG. 1F depicts a top view of retentate (122) and permeate (120) members of an exemplary TFF module with an alternative reservoir configuration. Again, ports 104 and 106 are seen. As noted above, recesses—such as the recesses 108 seen in FIG. 1B—provide a means to secure the components (retentate member 122, permeate member 120, and membrane 124) of the TFF device/membrane to one another during operation via, e.g., screws or other like fasteners. However, in alterative embodiments an adhesive, such as a pressure sensitive adhesive, or ultrasonic welding, solvent bonding, or a combination thereof may be used to couple the retentate member 122, permeate member 120, and membrane 124 together. Indeed, one of ordinary skill in the art given the guidance of the present disclosure can find yet other configurations for coupling the components of the TFF device, such as e.g., clamps, mated fittings disposed on the retentate and permeate members, and other such fasteners.

Again, there is one retentate port and one permeate/filtrate port on each “end” (e.g., the narrow edges) of this embodiment of a TFF device/module. The retentate and permeate/filtrate ports on the left side of the device/module will collect cells (flow path at 160) and medium (flow path at 170), respectively, for the same pass. Likewise, the retentate and permeate/filtrate ports on the right side of the device/module will collect cells (flow path at 160) and medium (flow path at 170), respectively, for the same pass. In this embodiment, the retentate is collected from ports 104 on the top surface of the TFF device, and filtrate is collected from ports 106 on the bottom surface of the device. The cells are maintained in the TFF flow channel above the membrane 124, while the filtrate (medium) flows through membrane 124 and then through ports 106. In FIG. 1F the retentate reservoirs are seen at 180, collecting retentate 160, and retentate reservoirs 180 comprise tube fittings (not shown) and tubes 190 which allow air or gas to enter the reservoirs to assist in cell growth, and/or allow medium or an exchange buffer to be added to retentate reservoirs 180. In FIG. 1F, the channel structure 102 can be seen on the bottom member 120 of the TFF device 100. However, in other embodiments, retentate and permeate/filtrate ports can reside on the same of the TFF device. Also seen in FIG. 1F is membrane or filter 124.

Medium exchange (during cell growth) or buffer exchange (during cell concentration or rendering the cells competent) is performed on the TFF device/module by adding fresh medium to growing cells (that is, refreshing medium to replace depleted nutrients) or by adding a desired buffer to the cells concentrated to a desired volume; for example, after the cells have been concentrated at least 20-fold, 30-fold, 40-fold, 50-fold, 60-fold, 70-fold, 80-fold, 90-fold, 100-fold, 150-fold, 200-fold or more. A desired exchange medium or exchange buffer is added to the cells either by addition to the retentate reservoir (e.g., to cells in retentate reservoir 130) or medium or buffer may be added through the membrane from the permeate/filtrate side and the process of passing the cells through the TFF device 100 is repeated until the cells have been grown to a desired optical density or concentrated to a desired volume in the exchange medium or buffer. This process can be repeated any number of desired times so as to achieve a desired level of exchange of the buffer and a desired volume of cells. As described in the Example 2, in the context of cell concentration, the exchange buffer may comprise, e.g., glycerol or sorbitol thereby rendering the cells competent for transformation in addition to decreasing the overall volume of the cell sample.

The TFF device 100 may be fabricated from any robust material in which channels and channel branches may be milled including stainless steel, silicon, glass, aluminum, or plastics including cyclic-olefin copolymer (COC), cyclo-olefin polymer (COP), polystyrene, polyvinyl chloride, polyethylene, polyamide, polyethylene, polypropylene, acrylonitrile butadiene, polycarbonate, polyetheretheketone (PEEK), poly(methyl methylacrylate) (PMMA), polysulfone, and polyurethane, and co-polymers of these and other polymers. If the TFF device/module is disposable, preferably it is made of plastic. In some embodiments, the material used to fabricate the TFF device/module is thermally-conductive so that the cell culture may be heated or cooled to a desired temperature. In certain embodiments, the TFF device is formed by precision mechanical machining, laser machining, electro discharge machining (for metal devices); wet or dry etching (for silicon devices); dry or wet etching, powder or sandblasting, photostructuring (for glass devices); or thermoforming, injection molding, hot embossing, or laser machining (for plastic devices) using the materials mentioned above that are amenable to this mass production techniques.

FIG. 1G depicts an alternative configuration of an assembled TFF device, where, like the other configurations, the retentate member and permeate member in combination form a channel structure with a membrane disposed between the retentate and permeate members; however in this configuration—in addition to the retentate reservoirs—there is a buffer or medium reservoir positioned between the retentate reservoirs, and a lower single filtrate or permeate reservoir. In the TFF device 1100 configuration shown in FIG. 1G, 1144 is the top or cover of the TFF device 1100, having three ports 1146, where there is a pipette tip 1148 disposed in the right-most port 1146. The top 1144 of the TFF device 1100 is adjacent to and in operation is coupled with a combined reservoir and retentate member structure 1150. Combined reservoir and retentate member structure 1150 comprises a top surface that is adjacent the top or cover 1144 of the TFF device, a bottom surface which comprises the retentate member 1122 of the TFF device, where the retentate member 1122 of the TFF device defines the upper portion of the flow channel (not shown) disposed on the bottom surface of the retentate member 1122 of the combined reservoir and retentate member structure 1150. Additionally, combined reservoir and retentate member structure 1150 comprises two retentate reservoirs 1180 and buffer or medium reservoir 1182.

The retentate reservoirs are fluidically coupled to the upper portion of the flow channel (e.g., the portion of the flow channel disposed in the retentate member), and the buffer or medium reservoir is fluidically coupled to the retentate reservoirs. Also seen in this assembled view of TFF device 1100 is membrane 1124, permeate member 1120 which, as described previously, comprises on its top surface the lower portion of the tangential flow channel (not shown), where the channel structures of the retentate member 1122 and permeate member 1120 (neither shown in this view) mate to form a single flow channel. Beneath and adjacent to permeate member 1120 is a gasket 1140, which is interposed between permeate member 1120 and a filtrate (or permeate) reservoir 1142. The permeate/filtrate reservoir 1142 is in fluid connection with the lower portion of the flow channel (e.g., the portion of the flow channel disposed in the permeate member) as a receptacle for the filtrate or permeate that is removed from the cell culture. In operation, top 1144, combined reservoir and retentate member structure 1150, membrane 1124, permeate member 1120, gasket 1140, and permeate/filtrate reservoir 1142 are coupled and secured together to be fluid- and air-tight. The assembled TFF device 1100 typically is from 4 to 25 cm in height, or from 5 to 20 cm in height, or from 7 to 15 cm in height; from 5 to 30 cm in length, or from 8 to 25 cm in length, or from 10 to 20 cm in length; and is from 3 to 15 cm in depth, or from 5 to 10 cm in depth. An exemplary TFF device is 11 cm in height, 12 cm in length, and 8 cm in depth. The retentate reservoirs, buffer or medium reservoir, and tangential flow channel-forming structures may be configured to be cooled to 4° C. for cell maintenance, and 30° C. for cell growth. The dimensions for the serpentine channel recited above, as well as the specifications and materials for the filter and the TFF device apply to the embodiment of the device shown in FIGS. 1G-1N. In embodiments including the present embodiment, up to 120 mL of cell culture can be grown and/or filtered, or up to 100 mL, 90 mL, 80 mL, 70 mL, 60 mL, 50 mL, 40 mL, 30 mL or 20 mL of cell culture can be grown and/or filtered.

FIG. 1H depicts a cross section of the long side of TFF device 1100, showing the same basic structures seen in the cross-sectional view of assembled TFF device 1100 depicted in FIG. 1G. Seen in this cross-sectional view is top or cover 1144, where the top 1144 has three ports (not seen) and where there is a pipette tip 1148 disposed in the right-most port. Again, the top 1144 of the TFF device 1100 is adjacent to and in operation is coupled with a combined reservoir and retentate member structure 1150. Combined reservoir and retentate member structure 1150 comprises a top surface that is adjacent the top or cover 1144 of the TFF device, a bottom surface which comprises the retentate member 1122 of the TFF device, where the retentate member 1122 of the TFF device defines the upper portion of the flow channel (not shown) disposed on the bottom surface of the retentate member 1122 of the combined reservoir and retentate member structure 1150. Additionally, combined reservoir and retentate member structure 1150 comprises two retentate reservoirs 1180 and buffer or medium reservoir 1182. The retentate reservoirs are fluidically coupled to the upper portion of the flow channel (e.g., the portion of the flow channel disposed in the retentate member), and the buffer or medium reservoir is fluidically coupled to the retentate reservoirs. Also seen in this assembled view of TFF device 1100 is membrane 1124, permeate member 1120 which, as described previously comprises on its top surface the lower portion of the tangential a flow channel (e.g., the portion of the flow channel disposed in the permeate member) (not shown), where the upper and lower flow channel structures (neither shown in this view) of the retentate member 1122 and permeate member 1120, respectively, mate to form a single tangential flow channel. Beneath and adjacent to permeate member 1120 is gasket 1140, which is interposed between the bottom surface of permeate member 1120 and a filtrate (or permeate) reservoir 1142. Filtrate reservoir 1142 collects the filtrate or permeate removed from the cell culture. In operation, top 1144, combined reservoir and retentate member structure 1150, membrane 1124, permeate member 1120, gasket 1140, and permeate/filtrate reservoir 1142 are coupled and secured together to be fluid- and air-tight.

FIG. 1I depicts a cross section of the short end side of TFF device 1100, also showing the same basic structures in cross-sectional view seen in the assembled TFF device 1100 depicted in FIG. 1G and the cross-sectional view of the long side of TFF device 1100 seen in FIG. 1H. Seen in this cross-sectional view is top or cover 1144. The ports are not seen; however, there is a pipette tip 1148 disposed in one port. Again, the bottom surface of top 1144 of the TFF device 1100 is adjacent to and in operation is coupled with a combined reservoir and retentate member structure 1150. Combined reservoir and retentate member structure 1150 comprises a top surface that is adjacent top or cover 1144 of the TFF device, a bottom surface which comprises the retentate member 1122 of the TFF device, where the retentate member 1122 of the TFF device defines on its lower surface the upper portion of the tangential flow channel (not shown). In this cross-sectional view of the end of TFF device 1100, only a single retentate reservoir 1180 can be seen. Also seen in this cross-sectional view of TFF device 1100 is membrane 1124, permeate member 1120 which, as described previously comprises on its top surface the lower portion of the tangential flow channel (not shown), where the upper and lower portions of the flow channel structures (neither shown in this view) of the retentate member 1122 and permeate member 1120, respectively, mate to form a single flow channel. In operation, the mated upper and lower portions of the tangential flow channel are separated by a membrane or filter. Beneath and adjacent to permeate member 1120 is gasket 1140, which is interposed between the bottom surface of permeate member 1120 and a filtrate (or permeate) reservoir 1142, which collects filtrate or permeate removed from the cell culture. In operation, top 1144, combined reservoir and retentate member structure 1150, membrane 1124, permeate member 1120, gasket 1140, and permeate/filtrate reservoir 1142 are coupled and secured together to be fluid- and air-tight.

FIG. 1J depicts a perspective cross-sectional view of the long side of TFF device 1100, similar to the cross-sectional view shown in FIG. 1H. Like FIGS. 1G-1I, the TFF device in FIG. 1J comprises top or cover 1144, where the top 1144 has three ports 1146 and where there is a pipette tip 1148 disposed in the right-most port 1146 and right-most retentate reservoir 1180. Again, the top 1144 of the TFF device 1100 is adjacent to and in operation is coupled with a combined reservoir and retentate member structure 1150. Combined reservoir and retentate member structure 1150 comprises a top surface that is adjacent the top or cover 1144 of the TFF device, a bottom surface which comprises the retentate member 1122 of the TFF device, where the retentate member 1122 of the TFF device defines the upper portion of the flow channel (not shown). Additionally, combined reservoir and retentate member structure 1150 comprises two retentate reservoirs 1180 and buffer or medium reservoir 1182. The retentate reservoirs are fluidically coupled to the upper portion of the flow channel, and the buffer or medium reservoir is fluidically coupled to the retentate reservoirs. Also seen in this assembled view of TFF device 1100 is membrane 1124 and permeate member 1120 which, as described previously, comprises on its top surface the lower portion of the tangential flow channel (not shown). The flow channel structures (neither shown in this view) of the retentate member 1122 and permeate member 1120 mate to form a single flow channel with a filter or membrane positioned between the upper and lower channel portions. Beneath and adjacent to permeate member 1120 is gasket 1140, which is interposed between the bottom surface of permeate member 1120 and a filtrate (or permeate) reservoir 1142. In operation, top 1144, combined reservoir and retentate member structure 1150, membrane 1124, permeate member 1120, gasket 1140, and permeate/filtrate reservoir 1142 are coupled and secured together to be fluid- and air-tight.

FIG. 1K depicts an exploded perspective view of TFF device 1100. In this configuration, 1144 is the top or cover of the TFF device 1100, having three ports 1146, where there is a pipette tip 1148 disposed in the left-most port 1146. The top 1144 of the TFF device 1100 is, in operation, coupled with a combined reservoir and retentate member structure 1150. Combined reservoir and retentate member structure 1150 comprises a top surface that, in operation, is adjacent the top or cover 1144 of the TFF device, a bottom surface which comprises the retentate member 1122 of the TFF device, where the retentate member 1122 of the TFF device defines the upper portion of the tangential flow channel (not shown). Combined reservoir and retentate member structure 1150 comprises two retentate reservoirs 1180 and buffer or medium reservoir 1182. The retentate reservoirs are fluidically coupled to the upper portion of the flow channel, and the buffer or medium reservoir is fluidically coupled to the retentate reservoirs.

Also seen in this exploded view of TFF device 1100 is permeate member 1120 which, as described previously comprises on its top surface the lower portion of the tangential flow channel 1102 (seen on the top surface of permeate member 1120), where the upper and lower portions of the channel structures of the retentate member 1122 and permeate member 1120, respectively, when coupled mate to form a single flow channel (the membrane that is interposed between the retentate member 1122 and permeate member 1120 in operation is not shown). Beneath permeate member 1120 is gasket 1140, which in operation is interposed between permeate member 1120 and a filtrate (or permeate) reservoir 1142. In operation, top 1144, combined reservoir and retentate member structure 1150, membrane (not shown), permeate member 1120, gasket 1140, and permeate/filtrate reservoir 1142 are coupled and secured together to be fluid- and air-tight. In FIG. 1K, fasteners are shown that can be used to couple the various structures (top 1144, combined reservoir and retentate member structure 1150, membrane (not shown), permeate member 1120, gasket 1140, and permeate/filtrate reservoir 1142) together. However, as an alternative to screws or other like fasteners, the various structures of TFF device 1100 can be coupled using an adhesive, such as a pressure sensitive adhesive; ultrasonic welding; or solvent bonding. Further, a combination of fasteners, adhesives, and/or welding types may be employed to couple the various structures of the TFF device. One of ordinary skill in the art given the guidance of the present disclosure could find yet other configurations for coupling the components of TFF device 1100, such as e.g., clamps, mated fittings, and other such fasteners.

FIG. 1L depicts combined reservoir and retentate member structure 1150, comprising two retentate reservoirs 1180 and buffer or medium reservoir 1182, as well as retentate member 1120, which is disposed on the bottom of combined reservoir and retentate member structure 1150. Retentate member 1122 of the TFF device defines the upper portion of the tangential flow channel (not shown) disposed on the bottom surface of the combined reservoir and retentate member structure 1150. FIG. 1M is a top-down view of the upper surface 1152 of combined reservoir and retentate member structure 1150, depicting the top of retentate reservoirs 1180 and buffer or medium reservoir 1182. The retentate reservoirs are fluidically coupled to the upper portion of the flow channel, and the buffer or medium reservoir is fluidically coupled to the retentate reservoirs. FIG. 1N is a bottom-up view of the lower surface of combined reservoir and retentate member structure 1150, showing the retentate member 1120 with the upper portion of the tangential flow channel 1102 disposed on the bottom surface of retentate member 1120. The flow channel 1102 disposed on the bottom surface of retentate member 1120 in operation is mated to the bottom portion of the tangential flow channel disposed on the top surface of the permeate member (not shown in this view, but see FIG. 1K), where the upper and lower portions of the flow channel structure mate to form a single flow channel.

FIG. 1O is an exemplary architecture diagram showing, along with Tables 1 and 2, one embodiment of pneumatics and volumes employed to concentrate cells in the TFF module, as well as perform buffer exchange. Looking at FIG. 1O, two retentate reservoirs are seen (RR1 and RR2), as is the buffer reservoir (located between the retentate reservoirs), and the permeate reservoir (located beneath the TFF flow channel assembly). There are two flow meters (FM1 and FM2), five solenoid 3-way valves, two pressure sensors, two proportional valves, a pump capable of delivering pressures of −5 to 30 psi and filters positioned in between the pneumatics and the reservoirs and where the 3-way solenoid valves vent to atmosphere. The designation NC is for “normally closed”, NO is for “normally open”, and C is “common”. Table 1 provides, for each step of the cell concentration process, the status of each valve shown in FIG. 1O and the pressure detected by pressure sensors 1 and 2. In Table 1, for the pump, 1=on, and 0=off. For the solenoid valves, 1+energized, and 0=de-energized. Table 2 provides, for each step of the cell concentration process, the volume in mL of liquid in each reservoir (i.e., both retentate reservoirs, the buffer reservoir and the permeate or filtrate reservoir). The process assumes that the initial cell culture sample is loaded into retentate reservoir 1 (RR1).

For growing E. coli cells, the TFF is chilled to 4° C. prior to loading the cell sample into RR1, and the cells are passed through the TFF flow channel with aeration (bubbling) in the retentate reservoirs. Once the proper OD is reached, the E. coli cells are concentrated and buffer exchange is performed to render the cells competent with, e.g., glycerol-containing buffer. For growing yeast cells, the TFF is heated to 30° C. for growth in the TFF device with aeration. Once the desired OD is reached, the yeast cells are conditioned with aeration and then are concentrated and resuspended in buffer, such as buffer containing lithium acetate and DTT (dithiothreitol) (or DTT/TCEP (tris(2-carboxyethyl)phosphine)) to render the yeast cells competent. In either example, the cells are loaded into the TFF device, electroporation buffer is loaded into the buffer reservoir. During concentration, electroporation buffer is added to the retentate reservoirs from the buffer reservoirs and the cells are both concentrated and rendered electrocompetent. During a “pass”, air pressure and flow rate are monitored. When fluid has been “pushed” into a reservoir, the flow rate spikes because fluid is no longer being pushed in the system and air begins flowing through the retentate channel, thus signaling the end of a pass. The process of transferring fluid from one reservoir to the other reservoir is a “pass”, and one to many passes may be performed to arrive at the proper buffer exchange and/or concentration desired (e.g., a concentration “round”). In some embodiments, fluid on the permeate side of the channel may be pulled across the membrane to assist in dislodging cells from the membrane on the retentate side of the membrane. After dislodging the cells, buffer may be added to one of the reservoirs and pressure applied to “sweep” the cells into the opposite reservoir.

In one embodiment, the TFF device or module constantly measures cell culture growth, and in some aspects, cell culture growth is measured via optical density (OD) of the cell culture in one or both of the retentate reservoirs and/or in the flow channel of the TFF device. Optical density may be measured continuously (kinetic monitoring) or at specific time intervals; e.g., every 5, 10, 15, 20, 30 45, or 60 seconds, or every 1, 2, 3, 4, 5, 6, 7, 8, 9, or 10 or so on minutes. Alternatively, OD can be measured at specific time intervals early in the cell growth cycle, and continuously after the OD of the cell culture reaches a set point OD. The TFF module is controlled by a processor, which can be programmed to measure OD constantly or at intervals as defined by a user. A script on, e.g., the reagent cartridge(s) may also specify the frequency for reading OD, as well as the target OD and target time. Additionally, a user manually can set a target time at which the user desires the cell culture hit a target OD. To accomplish reaching the target OD at the target time, the processor measures the OD of the growing cells, calculates the cell growth rate in real time, and predicts the time the target OD will be reached. The processor then automatically adjusts the temperature of the TFF module (and the cell culture) as needed. Lower temperatures slow growth, and higher temperatures increase growth. In addition, the processor may be programmed to inform a user of the progress of cell growth, buffer exchange, and/or cell concentration by altering the user via, e.g., cell phone or other personal digital device. Aside from OD, other properties of the cell culture can be measured, such as impedance of the culture, measurement of metabolic by-products or measurement of other cellular characteristics that correlate with the rate of growth of the cell culture.

FIGS. 1P-1X depict three alternative embodiments of a tangential flow filtration (TFF) device/module, where these embodiments have the advantage of a reduced footprint, in, e.g., an automated multi-module cell processing instrument. One embodiment comprises one permeate port and two retentate ports (see FIGS. 1P, 1Q, 1BB, and 1CC) and the other two embodiments feature two permeate ports and two retentate ports (see FIGS. 1R-1X and 1DD). FIG. 1P depicts a configuration of retentate member 1222 (on left), a membrane or filter 1224 (middle), and a permeate member 1220 (on the right) that is an alternative to that depicted in FIGS. 1C and 1D which are used in the cell concentration devices/modules of FIGS. 1E-1N. In the configurations shown in FIGS. 1P-1Y, the retentate member 1222 is no longer “upper” and the permeate member 1220 is no longer “lower”, as the retentate member 1222 and permeate member 1220 are instead coupled side-to-side as seen in FIGS. 1BB, 1CC and 1DD.

In FIG. 1P, retentate member 1222 comprises a tangential flow channel 1202, which has a serpentine configuration that initiates at one lower corner of retentate member 1222—specifically at retentate port 1228—traverses across and up then down and across retentate member 1222, ending in the other lower corner of retentate member 1222 at a second retentate port 1228. Also seen on retentate member 1222 is energy director 1291, which circumscribes the region where membrane or filter 1224 is seated. Energy director 1291 in this embodiment mates with and serves to facilitate ultrasonic wending or bonding of retentate member 1222 with permeate member 1220 via the energy director component on permeate member 1220. Also seen is membrane or filter 1224 having through-holes for retentate ports 1228, where the membrane is configured to seat within the circumference of energy directors 1291 between the retentate member 1222 and the permeate member 1220. Permeate member 1220 comprises, in addition to energy director 1291, through-holes for retentate port 1228 at each bottom corner (which mate with the through-holes for retentate ports 1228 at the bottom corners of membrane 1224 and retentate ports 1228 in retentate member 1222), as well as a tangential flow channel 1202 and a single permeate/filtrate port 1226 positioned at the top and center of permeate member 1220. The retentate member, membrane and permeate member in combination form a tangential flow assembly.

As in the tangential flow channel 102 configuration seen in FIG. 1B, the tangential flow channel 1202 structure in this embodiment has a serpentine configuration and an undulating geometry, although other geometries may be used. In some aspects, the length of the tangential flow channel is from 10 mm to 1000 mm, from 60 mm to 200 mm, or from 80 mm to 100 mm. In some aspects, the width of the channel structure is from 10 mm to 120 mm, from 40 mm to 70 mm, or from 50 mm to 60 mm. In some aspects, the cross section of the tangential flow channel 1202 is rectangular. In some aspects, the cross section of the tangential flow channel 1202 is 5 μm to 1000 μm wide and 5 μm to 1000 μm high, 300 μm to 700 μm wide and 300 μm to 700 μm high, or 400 μm to 600 μm wide and 400 μm to 600 μm high. In other aspects, the cross section of the tangential flow channel 1202 is circular, elliptical, trapezoidal, or oblong, and is 100 μm to 1000 μm in hydraulic radius, 300 μm to 700 μm in hydraulic radius, or 400 μm to 600 μm in hydraulic radius. Again, geometries are used that facilitate a large volume of filtering over a small area or footprint and the geometry must allow for the cell culture to be transferred back and forth through the flow channel.

FIG. 1Q is a side perspective view of a reservoir assembly 1250, which is similar to the combined reservoir and retentate member structure 1150 of FIG. 1G; however, in the embodiment of FIG. 1Q, the tangential flow assembly (comprising the retentate member, membrane and permeate member) is separate from the reservoir assembly. Instead, the reservoir assembly of FIG. 1Q is configured to be used with a retentate member, membrane and permeate member (tangential flow assembly) such as that seen in FIG. 1P. Reservoir assembly 1250 comprises retentate reservoirs 1252 on either side of a single permeate reservoir 1254. Retentate reservoirs 1252 are used to contain the cells and medium as the cells are transferred through the flow channel and into the retentate reservoirs during cell concentration and/or growth. Permeate reservoir 1254 is used to collect the filtrate fluids removed from the cell culture during cell concentration or old buffer or medium during cell growth. There is not a buffer reservoir equivalent to that of buffer or medium reservoir 1182 (seen in FIGS. 1G, 1H and 1J-1M). Instead in the embodiment depicted in FIGS. 1P-1DD, buffer or medium is supplied to the retentate member from a reagent reservoir separate from the TFF module. Additionally seen in FIG. 1Q are grooves 1232 to accommodate pneumatic ports (not seen), a single permeate/filtrate port 1226, and retentate port through-holes 1228. The retentate reservoirs are fluidically coupled to the retentate ports 1228, which in turn are fluidically coupled to the portion of the tangential flow channel disposed 1202 in the retentate member 1222 (not shown but see FIG. 1P). The permeate reservoir is fluidically coupled to the single permeate port 1226 which in turn is fluidically coupled to the portion of the tangential flow channel disposed in permeate member (not shown but see FIG. 1P), where the portions of the mated tangential flow channels are bifurcated by the membrane (not shown). In embodiments including the present embodiment, up to 120 mL of cell culture can be grown and/or filtered, or up to 100 mL, 90 mL, 80 mL, 70 mL, 60 mL, 50 mL, 40 mL, 30 mL or 20 mL of cell culture can be grown and/or concentrated.

As described above, the overall work flow for cell growth comprises loading a cell culture to be grown into a first retentate reservoir 1252, optionally bubbling air or an appropriate gas through the cell culture, passing or flowing the cell culture through the first retentate port 1228 then tangentially through the TFF channel structure while collecting medium or buffer through one (or both, depending on the embodiment) of the permeate/filtrate ports 1226, collecting the cell culture through a second retentate port 1228 into a second retentate reservoir 1252, optionally adding additional or different medium to the cell culture and optionally bubbling air or gas through the cell culture, then repeating the process, all while measuring, e.g., the optical density of the cell culture in the retentate reservoirs continuously or at desired intervals. Measurements of optical densities (OD) at programmed time intervals are accomplished using a 600 nm Light Emitting Diode (LED) that has been columnated through an optic into the retentate reservoir(s) containing the growing cells. In the channel structure, the membrane bifurcating the flow channels retains the cells on one side of the membrane (the retentate side) and allows unwanted medium or buffer to flow across the membrane into a filtrate or permeate side of the device. Bubbling air or other appropriate gas through the cell culture both aerates and mixes the culture to enhance cell growth. During the process, medium that is removed during the flow through the channel structure is removed through the permeate/filtrate port(s) 1226 and is collected in the permeate reservoir 1254. Alternatively, cells can be grown in one reservoir with bubbling or agitation without passing the cells through the TFF channel from one reservoir to the other.

The overall work flow for cell concentration using the TFF device/module involves flowing a cell culture or cell sample tangentially through the channel structure. As with the cell growth process, the membrane bifurcating the flow channels retains the cells on one side of the membrane (retentate) and allows unwanted medium or buffer (permeate) to flow across the membrane into a permeate side (e.g., permeate member 1220) of the device. In this process, a fixed volume of cells in medium or buffer is driven through the device until the cell sample is collected into one of the retentate reservoirs 1252, and the medium/buffer that has passed through the membrane is collected through one (or both, depending on the embodiment) of the permeate/filtrate port(s) 1226 into permeate reservoir 1254.

FIG. 1R, like FIG. 1P, depicts a configuration of a retentate member 1222 (at left), a membrane or filter 1224 (middle), and a permeate member 1220 (at right) that also is an alternative to that depicted in FIGS. 1C and 1D. Again, in the configurations shown in FIGS. 1P-1DD, the retentate member 1222 is no longer “upper” and the permeate/filtrate member 1220 is no longer “lower”, as the retentate member 1222 and permeate/filtrate member 1220 are coupled side-to-side as seen in FIGS. 1BB-1DD. In FIG. 1R, retentate member 1222 comprises a tangential flow channel 1202, which has a serpentine configuration that initiates at one lower corner of retentate member 1222—specifically at retentate port 1228—traverses across and up then down and across retentate member 1222, ending in the other lower corner of retentate member 1222 at a second retentate port 1228. Also seen on retentate member 1222 are energy directors 1291, which circumscribe the region where membrane or filter 1224 is seated, as well as interdigitate between areas of the channel. Energy directors 1291 in this embodiment—as with the embodiment in FIG. 1P—mate with and serve to facilitate ultrasonic welding or bonding of retentate member 1222 with permeate/filtrate member 1220 via the energy director component 1291 on permeate/filtrate member 1220 (at right). Additionally, pin slot alignment elements 1292 are depicted.

Membrane or filter 1224 is seen at center in FIG. 1R, where member 1224 is configured to seat within the circumference of energy directors 1291 between the retentate member 1222 and the permeate member 1220. Permeate member 1220 comprises, in addition to energy director 1291, through-holes for retentate ports 1228 at each bottom corner (which mate with the through-holes for retentate ports 1228 at the bottom corners of retentate member 1222), as well as a tangential flow channel 1202 and two permeate ports 1226 positioned at the top and center of permeate member 1220. As in the tangential flow channel configuration seen in FIGS. 1B and 1P, the tangential flow channel 1202 structure in this embodiment has a serpentine configuration and an undulating geometry, although other geometries may be used. As described above, the length of the tangential flow channel is from 10 mm to 1000 mm, from 60 mm to 200 mm, or from 80 mm to 100 mm. In some aspects the width of the channel structure is from 10 mm to 120 mm, from 40 mm to 70 mm, or from 50 mm to 60 mm. In some aspects the cross section of the tangential flow channel 1202 is rectangular, and in some aspects the cross section of the tangential flow channel 1202 is 5 μm to 1000 μm wide and 5 μm to 1000 μm high, 300 μm to 700 μm wide and 300 μm to 700 μm high, or 400 μm to 600 μm wide and 400 μm to 600 μm high. In other aspects, the cross section of the tangential flow channel 1202 is circular, elliptical, trapezoidal, or oblong, and is 100 μm to 1000 μm in hydraulic radius, 300 μm to 700 μm in hydraulic radius, or 400 μm to 600 μm in hydraulic radius.

FIG. 1S is a side view (left) and a side perspective view (right) of a reservoir assembly 1250, which is similar to the reservoir assembly 1250 of FIG. 1Q. Like the embodiment of FIG. 1Q, the tangential flow assembly including the retentate member, membrane and permeate member is separate from the reservoir assembly (in contrast to the embodiment shown in FIGS. 1J-1N). In both views of reservoir assembly 1250, the reservoir assembly comprises retentate reservoirs 1252 on either side of a single permeate reservoir 1254. Retentate reservoirs 1252 are used to contain the cells and medium as the cells are transferred through the cell concentration/growth device or module and into the retentate reservoirs during cell concentration and/or growth. Permeate reservoir 1254 is used to collect the filtrate fluids (e.g., waster) removed from the cell culture during cell concentration or old buffer or medium during cell growth. As with the reservoir embodiment seen in FIG. 1Q, there is not a buffer reservoir equivalent to that of buffer or medium reservoir 1182 (seen in FIGS. 1G, 1H and 1J-1M). Instead in the embodiment depicted in FIGS. 1P-1DD, buffer or medium is supplied to the retentate member from a reagent reservoir separate from the device module.

Additionally seen in FIG. 1S in reservoir assembly 1250 there are two permeate ports 1226, and retentate port through-holes 1228. The retentate reservoirs are fluidically coupled to the retentate ports 1228, which in turn are fluidically coupled to the portion of the tangential flow channel disposed in the retentate member (not shown). The permeate reservoirs are fluidically coupled to the permeate ports 1226 which in turn are fluidically coupled to the portion of the tangential flow channel disposed in permeate member (not shown), where the portions of the tangential flow channels are bifurcated by membrane 1224 (not shown). In embodiments including the present embodiment, up to 120 mL of cell culture can be grown and/or filtered, or up to 100 mL, 90 mL, 80 mL, 70 mL, 60 mL, 50 mL, 40 mL, 30 mL or 20 mL of cell culture can be grown and/or concentrated.

FIG. 1T is similar to FIGS. 1P and 1R; however, FIG. 1T does not show the retentate member, membrane and permeate member, but instead shows the retentate member 1222 (top), permeate member 1220 (middle) and a tangential flow assembly 1210 (bottom) comprising the retentate member 1222, membrane 1224 (not seen in FIG. 1T), and permeate member 1220 (also not seen). In FIG. 1T, retentate member 1222 comprises a tangential flow channel 1202, which has a serpentine configuration that initiates at one lower corner of retentate member 1222—specifically at retentate port 1228—traverses across and up then down and across retentate member 1222, ending in the other lower corner of retentate member 1222 at a second retentate port 1228. Also seen on retentate member 1222 are energy directors 1291, which circumscribe the region where a membrane or filter (not seen in this FIG. 1T) is seated, as well as interdigitate between areas of channel 1202. Energy directors 1291 in this embodiment—as with the embodiment in FIGS. 1P and 1R—mate with and serve to facilitate ultrasonic welding or bonding of retentate member 1222 with permeate/filtrate member 1220 via the energy director component 1291 on permeate/filtrate member 1220 (at right). Additionally, countersinks 1223 can be seen, two on the bottom one at the top middle of retentate member 1222. Countersinks 1223 are used to couple and tangential flow assembly 1210 to a reservoir assembly (not seen in this FIG. 1T but see FIG. 1V).

Permeate/filtrate member 1220 is seen in the middle of FIG. 1T and comprises, in addition to energy director 1291, through-holes for retentate ports 1228 at each bottom corner (which mate with the through-holes for retentate ports 1228 at the bottom corners of retentate member 1222), as well as a tangential flow channel 1202 and two permeate/filtrate ports 1226 positioned at the top and center of permeate member 1220. As with the tangential flow channel configuration seen and described previously, the tangential flow channel 1202 structure in this embodiment has a serpentine configuration and an undulating geometry, although other geometries may be used. Permeate member 1220 also comprises countersinks 1223, coincident with the countersinks 1223 on retentate member 1220. As described above, the length of the tangential flow channel is from 10 mm to 1000 mm, from 60 mm to 200 mm, or from 80 mm to 100 mm. In some aspects the width of the channel structure is from 10 mm to 120 mm, from 40 mm to 70 mm, or from 50 mm to 60 mm. In some aspects the cross section of the tangential flow channel 1202 is rectangular, and in some aspects the cross section of the tangential flow channel 1202 is 5 μm to 1000 μm wide and 5 μm to 1000 μm high, 300 μm to 700 μm wide and 300 μm to 700 μm high, or 400 μm to 600 μm wide and 400 μm to 600 μm high. In other aspects, the cross section of the tangential flow channel 1202 is circular, elliptical, trapezoidal, or oblong, and is 100 μm to 1000 μm in hydraulic radius, 300 μm to 700 μm in hydraulic radius, or 400 μm to 600 μm in hydraulic radius.

The bottom figure of FIG. 1T is a tangential flow assembly 1210 comprising the retentate member 1222 and permeate member 1220 seen in this FIG. 1T. In this view, retentate member 1222 is “on top” of the view, a membrane (not seen in this view of the assembly) would be adjacent and under retentate member 1222 and permeate member 1220 (also not seen in this view of the assembly) is adjacent to and beneath the membrane. Again countersinks 1223 are seen, where the countersinks in the retentate member 1222 and the permeate member 1220 are coincident and configured to mate with threads or mating elements for the countersinks disposed on a reservoir assembly (not seen in FIG. 1T but see FIG. 1V).

FIG. 1U is a cross-sectional side view of an embodiment of the tangential flow assembly depicted at left of FIG. 1T. Looking from top to bottom is retentate member 1222 comprising tangential flow channel 1202 and energy directors 1291, a membrane 1224, an over mold 1219, which surrounds tangential flow channel 1202 disposed in permeate member 1220, and energy directors 1291 in permeate member 1220. Over mold 1219 here is added to permeate member 1220 but may be instead disposed on retentate member 1222 or be disposed on both permeate member 1220 and retentate member 1222. Over mold 1219 serves the purpose of ensuring a fluid-tight coupling of the two sides of tangential flow channel 1202 and membrane 1224. Over mold 1219 may be comprised of a compressible material such as neoprene rubber, silicone rubber, polyurethane rubber, buna-n-rubber, EPDM rubber, SBR rubber, natural rubber, VITON® fluoroelastomer rubber, aflas rubber, santoprene rubber, butyl rubber, kalrez rubber or fluorosilicone rubber, with a durometer of 10-90, or from 20-80, or from 30-70 and may be from 100 μm to 800 μm thick, or from 200 μm to 700 μm thick, or from 300 μm to 600 μm thick with a, e.g., 10% additional thickness to allow for compression. Over mold 1219 may be added to the retentate or permeate members by first injection molding the retentate or permeate member, then injection molding the over mold in designated areas over the injection molded retentate and/or permeate members.

FIG. 1V shows front perspective (right) and rear perspective (left) views of a reservoir assembly 1250 configured to be used with the tangential flow assembly 1210 seen in FIG. 1T. Seen in the front perspective view (e.g., “front” being the side of reservoir assembly 1250 that is coupled to the tangential flow assembly 1210 seen in FIG. 1T) are retentate reservoirs 1252 on either side of permeate reservoir 1254. As in the embodiments shown in FIGS. 1Q and 1S, there is no buffer reservoir or reserve; instead a buffer reservoir is configured to be apart from the TFF module. Also seen are permeate ports 1226, retentate ports 1228, and three threads or mating elements 1225 for countersinks 1223 (countersinks 1223 not seen in this FIG. 1V). Threads or mating elements 1225 for countersinks 1223 are configured to mate or couple the tangential flow assembly 1210 (seen in FIG. 1T) to reservoir assembly 1250. Alternatively or in addition, fasteners, sonic welding or heat stakes may be used to mate or couple the tangential flow assembly 1210 to reservoir assembly 1250. In addition is seen gasket 1245 covering the top of reservoir assembly 1250. Gasket 1245 is described in detail in relation to FIG. 1AA. At left in FIG. 1V is a rear perspective view of reservoir assembly 1250, where “rear” is the side of reservoir assembly 1250 that is not coupled to the tangential flow assembly. Seen are retentate reservoirs 1252, permeate reservoir 1254, and gasket 1245.

FIG. 1W is a cross-sectional view of the bottom of a retentate reservoir 1252 with a portion of a pipette tip 1205 disposed therein, a cross section of retentate port channel 1229, retentate port 1228, permeate member 1220, membrane 1224, and retentate member 1222. In addition, a cross section of O-ring 1231 is seen surrounding retentate port 1228 where retentate port 1228 in permeate member 1220 is coupled to reservoir assembly 1250.

FIG. 1X is a cross-sectional side view of the reservoir assembly depicted in FIG. 1V coupled to the tangential flow assembly depicted in FIG. 1T. Seen moving from left to right are retentate member 1222, membrane 1224, permeate member 1220, retentate reservoir 1252 with pipette tip 1205 disposed therein, retentate port 1228, O-ring 1231 and retentate port channel 1229. Note that the bottom of retentate reservoir 1252 is asymmetrically sloped to aid in recovering all liquid in retentate reservoir 1252.

FIG. 1Y depicts a top-down view of the reservoir assemblies 1250 shown in FIGS. 1Q, 1S and 1V. FIG. 1Z depicts a cover 1244 for reservoir assembly 1250 shown in FIGS. 1Q, 1S, 1V and 1AA depicts a gasket 1245 that in operation is disposed on cover 1244 of reservoir assemblies 1250 shown in FIGS. 1Q, 1S and 1V. FIG. 1Y is a top-down view of reservoir assembly 1250, showing the tops of the two retentate reservoirs 1252, one on either side of permeate reservoir 1254. Also seen are grooves 1232 that will mate with a pneumatic port (not shown), and fluid channels 1234 that reside at the bottom of retentate reservoirs 1252, which fluidically couple the retentate reservoirs 1252 with the retentate ports 1228 (not shown), via the through-holes for the retentate ports in permeate member 1220 and membrane 1224 (also not shown). FIG. 1Z depicts a cover 1244 that is configured to be disposed upon the top of reservoir assembly 1250. Cover 1244 has round cut-outs at the top of retentate reservoirs 1252 and permeate/filtrate reservoir 1254. Again at the bottom of retentate reservoirs 1252 fluid channels 1234 can be seen, where fluid channels 1234 fluidically couple retentate reservoirs 1252 with the retentate ports 1228 (not shown). Also shown are three pneumatic ports 1230 for each retentate reservoir 1252 and permeate/filtrate reservoir 1254. FIG. 1AA depicts a gasket 1245 that is configures to be disposed upon the cover 1244 of reservoir assembly 1250. Seen are three fluid transfer ports 1242 for each retentate reservoir 1252 and for permeate/filtrate reservoir 1254. Again, three pneumatic ports 1230, for each retentate reservoir 1252 and for permeate/filtrate reservoir 1254, are shown.

FIG. 1BB depicts an embodiment of an assembled TFF module 1200. Again, note that in this embodiment of a TFF module the retentate member 1222 is no longer “upper”, and the permeate member 1220 is no longer “lower”, as the retentate member 1222 and permeate member 1220 are coupled side-to-side with membrane 1224 sandwiched between retentate member 1222 and permeate member 1220. Also, retentate member 1222, membrane member 1224, and permeate member 1220 are coupled side-to-side with reservoir assembly 1250. Seen are two retentate ports 1228, which couple the tangential flow channel 1202 in retentate member 1222 to the two retentate reservoirs (not shown), and one permeate port 1226, which couples the tangential flow channel 1202 in permeate member 1220 to the permeate reservoir (not shown). Also seen is tangential flow channel 1202, which is formed by the mating of retentate member 1222 and permeate member 1220, with membrane 1224 sandwiched between and bifurcating tangential flow channel 1202. Energy director 1291 is also present, which in this FIG. 1BB has been used to ultrasonically weld or couple retentate member 1222 and permeate/filtrate member 1220, surrounding membrane 1224. Cover 1244 can be seen on top of reservoir assembly 1250, and gasket 1245 is disposed upon cover 1244. Gasket 1245 engages with and provides a fluid-tight seal and pneumatic connections through a pneumatic actuator with fluid transfer ports 1242 and pneumatic ports 1230, respectively.

FIG. 1CC depicts, on the left, an exploded view of the TFF module 1200 shown in FIG. 1BB. Seen are components reservoir assembly 1250, a cover 1244 to be disposed on reservoir assembly 1250, a gasket 1245 to be disposed on cover 1244, retentate member 1222, membrane or filter 1224, and permeate member 1220. Also seen is permeate port 1226, which mates with permeate port 1226 on permeate reservoir 1254, as well as two retentate ports 1228, which mate with retentate ports 1228 on retentate reservoirs 1252 (where only one retentate reservoir 1252 can be seen clearly in this FIG. 1CC). Also seen are through-holes for retentate ports 1228 in membrane 1224 and permeate member 1220. FIG. 1CC depicts on the left the assembled TFF module 1200 showing length, height, and width dimensions. The assembled TFF device 1200 typically is from 50 to 175 mm in height, or from 75 to 150 mm in height, or from 90 to 120 mm in height; from 50 to 175 mm in length, or from 75 to 150 mm in length, or from 90 to 120 mm in length; and is from 30 to 90 mm in depth, or from 40 to 75 mm in depth, or from about 50 to 60 mm in depth. An exemplary TFF device is 110 mm in height, 120 mm in length, and 55 mm in depth.

FIG. 1DD depicts, on the left, an assembled view of the TFF module 1200 without retentate member 1222, and on the right, an assembled view of the TFF module 1200 with retentate member 1222. FIGS. 1BB and 1CC differ from FIG. 1DD in that the embodiments shown in FIGS. 1BB and 1CC have a single permeate port 1226 and the embodiment shown in FIG. 1DD has two permeate ports 1226. Seen are components reservoir assembly 1250, a gasket 1245 to be disposed on reservoir assembly 1250, retentate member 1222, membrane or filter 1224, and, only seen as a layer beneath membrane 1224, permeate member 1220. Also seen are permeate ports 1226 (seen at right), which mate with permeate ports 1226 on permeate reservoir 1254 (not seen), as well as two retentate ports 1228, which mate with retentate ports 1228 on retentate reservoirs 1252 (where only one retentate reservoir 1252 can be seen clearly in this FIG. 1DD). Pin slot alignment elements are seen at 1292. Also seen are through-holes for retentate ports 1228 in permeate/filtrate member 1220. As with FIG. 1CC, right, the left the assembled TFF module 1200 in FIG. 1DD typically is from 50 to 175 mm in height, or from 75 to 150 mm in height, or from 90 to 120 mm in height; from 50 to 175 mm in length, or from 75 to 150 mm in length, or from 90 to 120 mm in length; and is from 30 to 90 mm in depth, or from 40 to 75 mm in depth, or from about 50 to 60 mm in depth. An exemplary TFF device is 110 mm in height, 120 mm in length, and 55 mm in depth.

Like in other embodiments described herein, the TFF device or module depicted in FIGS. 1P-1DD can constantly measure cell culture growth, and in some aspects cell culture growth is measured via optical density (OD) of the cell culture in one or both of the retentate reservoirs and/or in the flow channel of the TFF device. Optical density may be measured continuously (real-time monitoring) or at specific time intervals; e.g., every 5, 10, 15, 20, 30 45, or 60 seconds, or every 1, 2, 3, 4, 5, 6, 7, 8, 9, or 10 or so on minutes. Further, the TFF module can adjust growth parameters (temperature, aeration) to have the cells at a desired optical density at a desired time.

FIG. 1EE is an exemplary pneumatic block diagram suitable for the TFF module depicted in FIGS. 1P-1DD. The pump is connected to two solenoid valves (SV3 and SV4) delivering positive pressure (P) or negative pressure (V). The two solenoid valves SV3 and SV4 couple the pump to the manifold arm, and two solenoid valves, SV1 and SV2, are connected to retentate reservoirs RR1 and RR2, respectively. There is a proportional valve (PV1 and PV2) and a pressure sensor (Pressure Sensors 1 and 2) positioned between the pump and each of solenoid valves SV1 and SV2. The pressure sensors and prop valves work in concert in a feedback loop to maintain a required pressure. Flow meters FM1 and FM2 are positioned between solenoid valve SV1 and retentate reservoir RR1 and solenoid valve SV2 and retentate reservoir RR2, respectively.

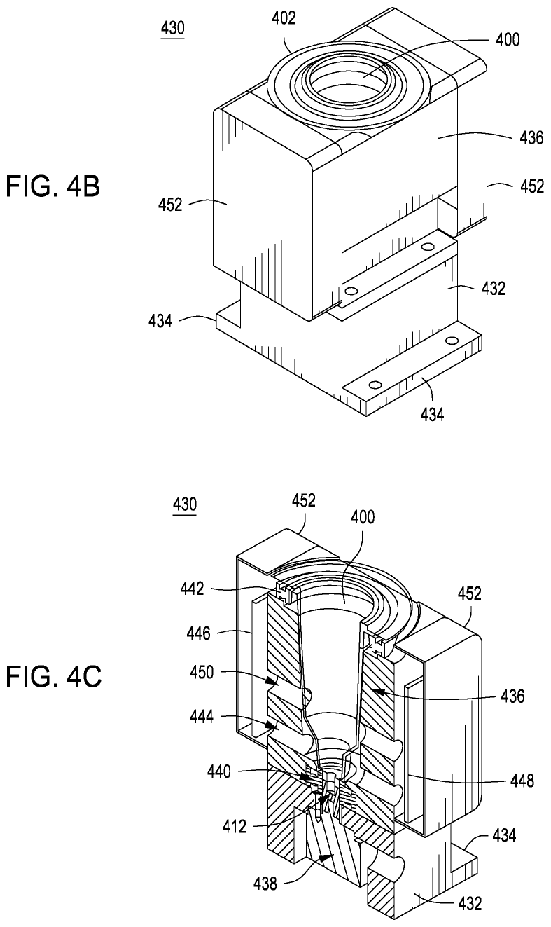

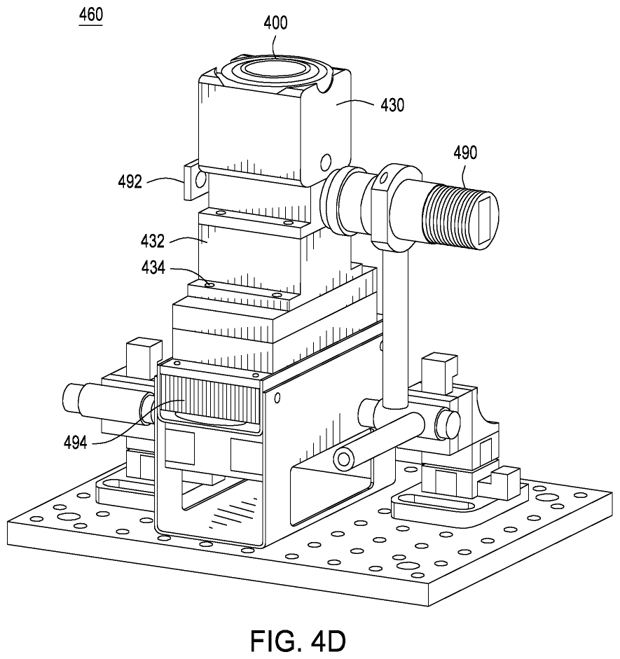

In a summary of steps for concentrating E. coli cells, cells that have been grown to a desired OD are transferred from, e.g., a cell growth device such as seen in FIGS. 4A-4D into retentate reservoir 1 (RR1). Alternatively, if the cells are grown in the TFF device itself, no transfer is necessary. In the first concentration round, initially pressure is applied to RR1 and RR2 individually, and then a different pressure is applied to each retentate reservoir simultaneously. The cells are passed through the TFF between reservoirs until the cell culture has been concentrated to a desired volume. RR1 is then loaded with 3ML buffer and RR2 is loaded with 15 mL buffer. The buffer from RR2 is transferred to RR1 to mix the cells and buffer in RR1, and a second concentration round is performed. The processes of concentration passes and buffer exchange are repeated until buffer exchange is complete and the cells have been concentrated to a desired volume. In the elution concentration round, pressure is applied to the two retentate reservoirs individually and the cells are lifted from the permeate membrane between each concentration pass and pulled in the direction of the previous concentration pass. Concentration passes are performed until a desired volume is attained and the cells are then swept into one of the retentate reservoirs. See Table 3 for the system state program for the system shown in FIG. 1EE.

In the present system, the flow meter that is coupled directly to the retentate reservoir to which the cell culture is being transferred is monitored to determine when the cells have been thoroughly transferred to that reservoir. That is, flow meter FM2 is read to ascertain whether the cell culture has been completely transferred to RR2, rather than FM1 being monitored to ascertain whether the cell culture has been entirely transferred from RR1. This practice is different from how such monitoring is accomplished typically. The reason behind this practice is that at times the volume of the cell culture is quite small and a good deal of the culture may have evacuated a retentate reservoir, but reside primarily within the TFF flow channel. By monitoring the flow meter coupled to the retentate reservoir to which the cell culture is being transferred (again, monitoring FM1 when RR1 is the retentate reservoir to which the cell culture is being transferred, and monitoring FM2 when RR2 is the retentate reservoir to which the cell culture is being transferred), one can detect when the entirety of the cell culture has been transferred from the transferring reservoir, through the flow channel and into the receiving reservoir.

### The Automated Multi-Module Cell Processing Instrument

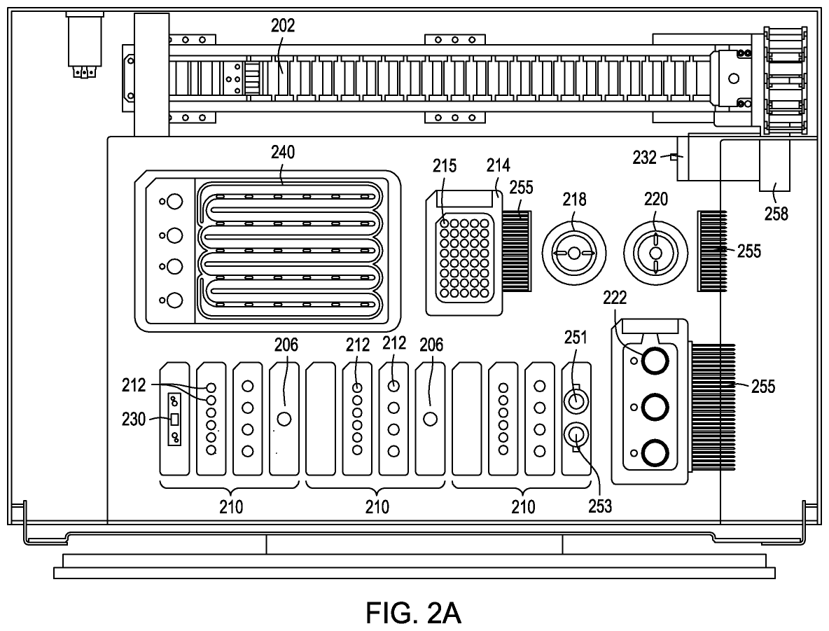

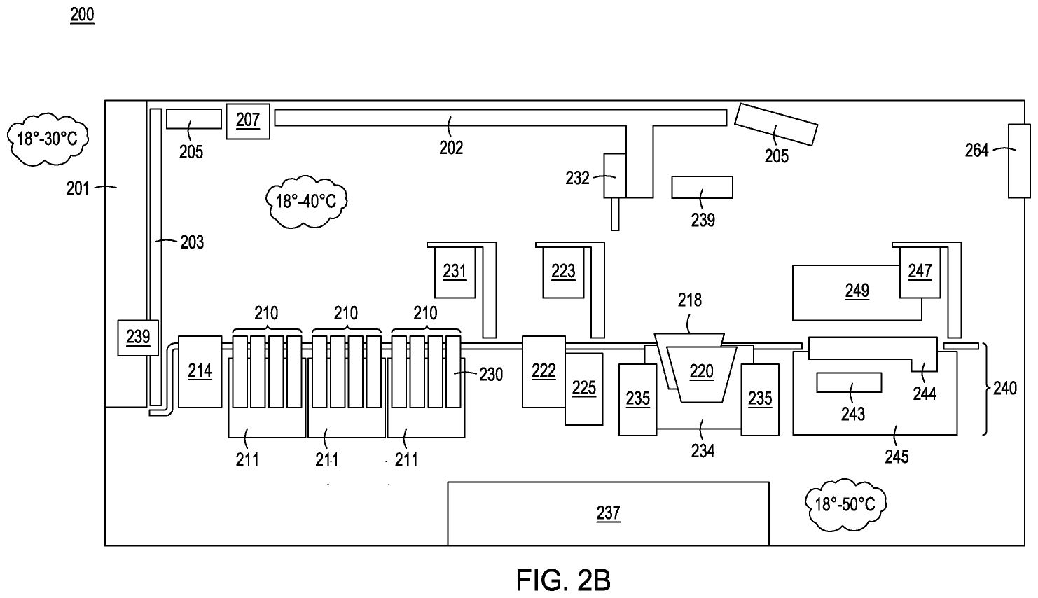

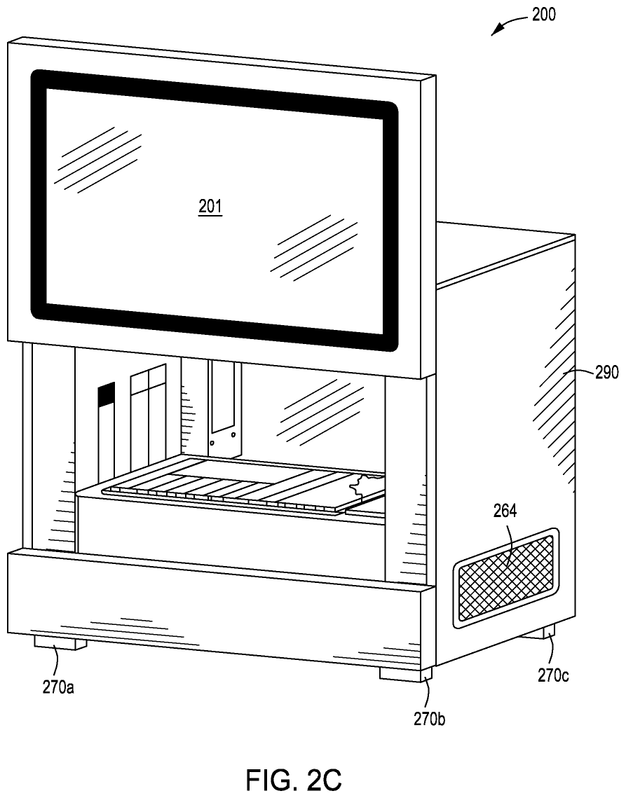

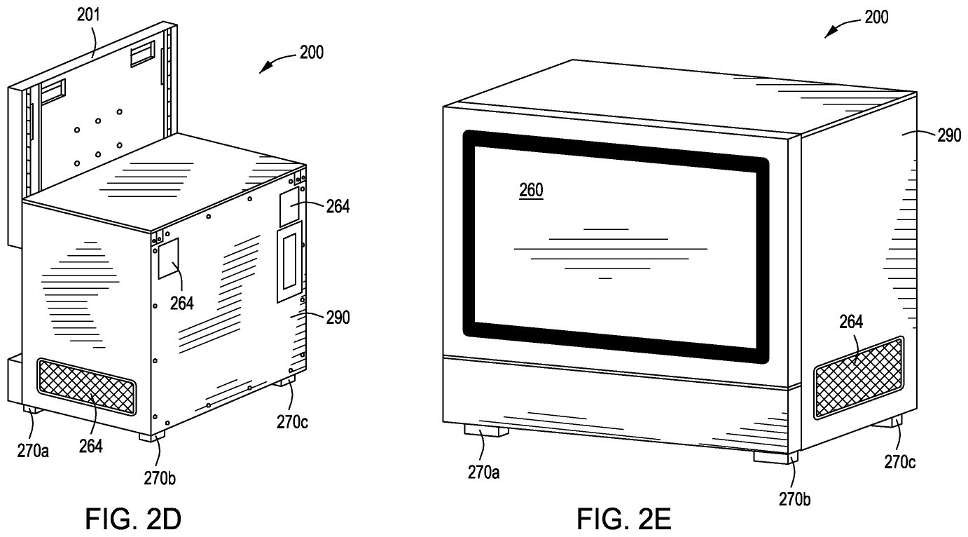

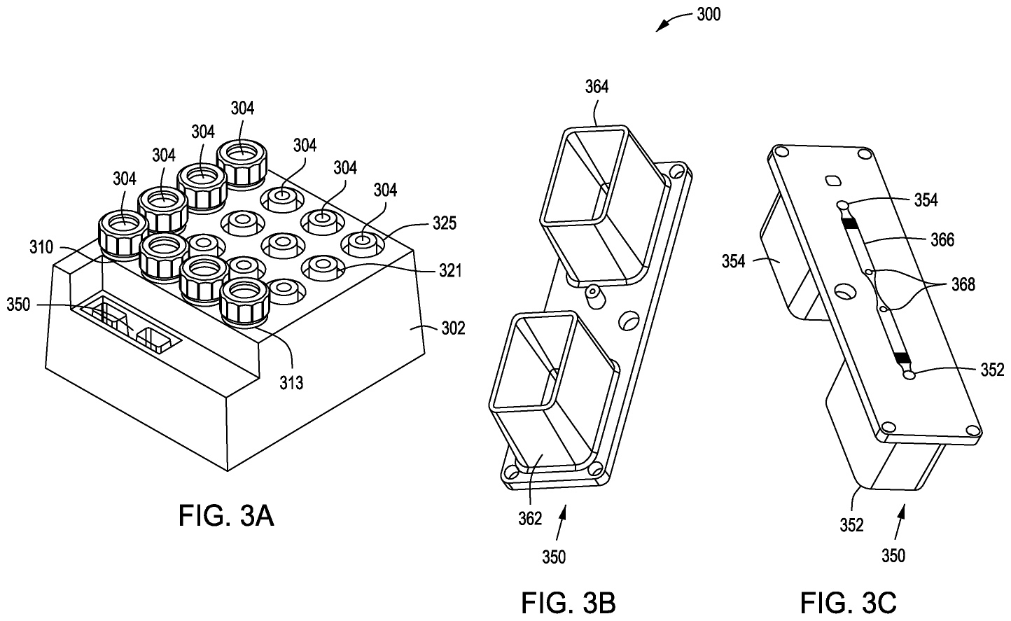

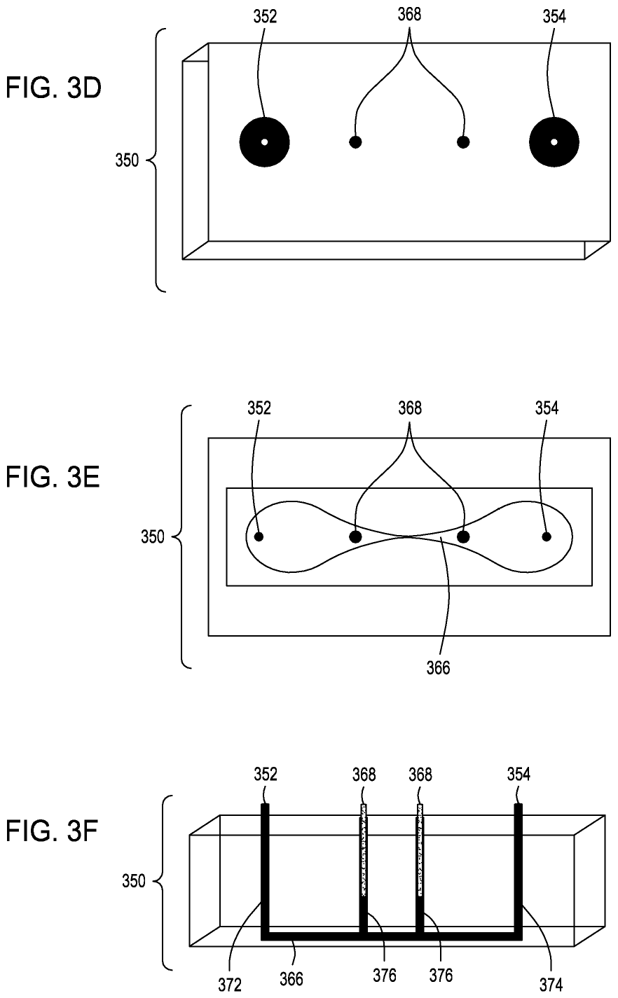

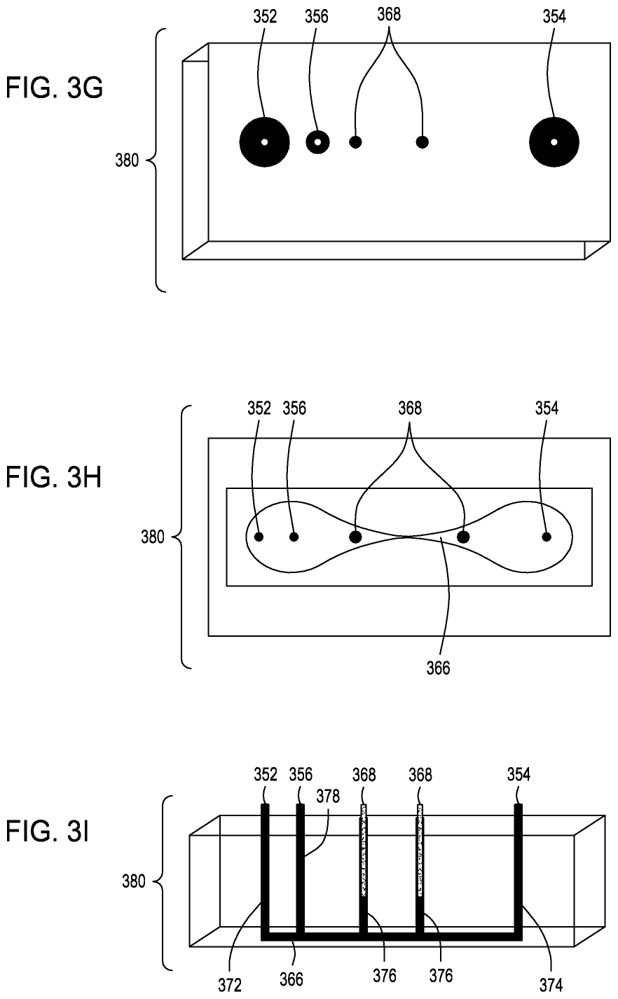

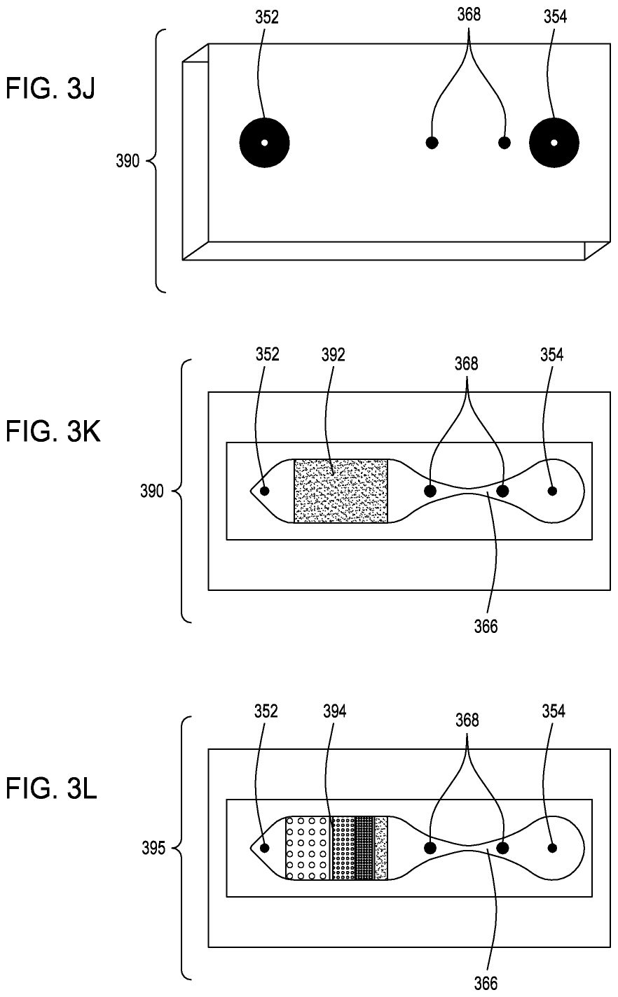

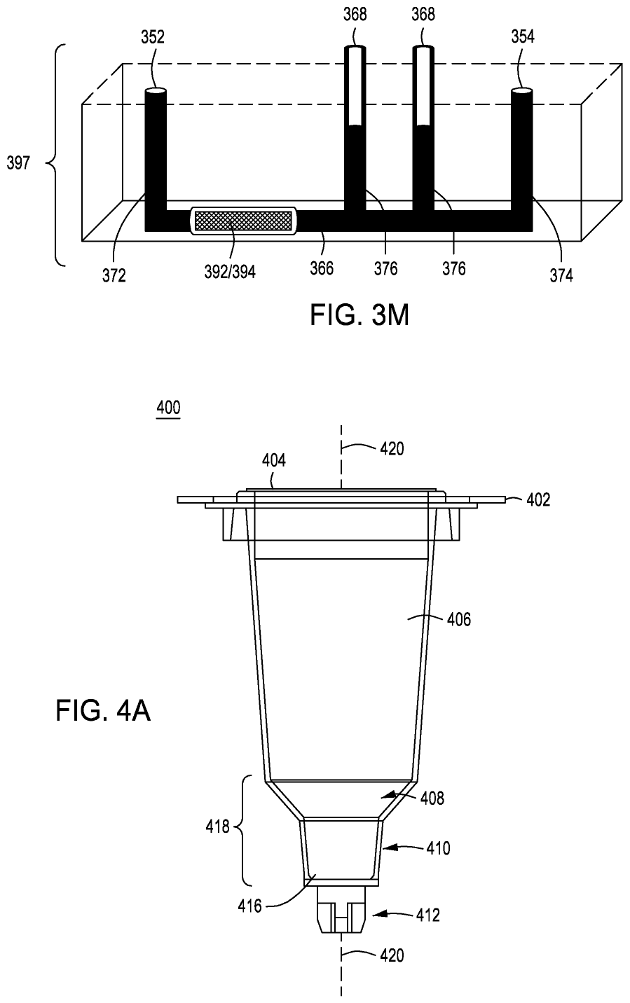

FIG. 2A depicts an exemplary automated multi-module cell processing instrument 200 to, e.g., perform one of the exemplary workflows described infra, comprising one or more tangential flow filtration modules as described herein. The instrument 200, for example, may be and preferably is designed as a stand-alone desktop instrument for use within a laboratory environment. The instrument 200 may incorporate a mixture of reusable and disposable components for performing the various integrated processes in conducting automated genome cleavage and/or editing in cells without human intervention. Illustrated is a gantry 202, providing an automated mechanical motion system (actuator) (not shown) that supplies XYZ axis motion control to, e.g., an automated (i.e., robotic) liquid handling system 258 including, e.g., an air displacement pipettor 232 which allows for cell processing among multiple modules without human intervention. In some automated multi-module cell processing instruments, the air displacement pipettor 232 is moved by gantry 202 and the various modules and reagent cartridges remain stationary; however, in other embodiments, the liquid handling system 258 may stay stationary while the various modules and reagent cartridges are moved. Also included in the automated multi-module cell processing instrument 200 are reagent cartridges 210 comprising reservoirs 212 and transformation module 230 (e.g., a flow-through electroporation device as described in detail in relation to FIGS. 3A-3M), as well as wash reservoirs 206, cell input reservoir 251 and cell output reservoir 253. The wash reservoirs 206 may be configured to accommodate large tubes, for example, wash solutions, or solutions that are used often throughout an iterative process. Although two of the reagent cartridges 210 comprise a wash reservoir 206 in FIG. 2A, the wash reservoirs instead could be included in a wash cartridge where the reagent and wash cartridges are separate cartridges. In such a case, the reagent cartridge 210 and wash cartridge 204 may be identical except for the consumables (reagents or other components contained within the various inserts) inserted therein.

In some implementations, the reagent cartridges 210 are disposable kits comprising reagents and cells for use in the automated multi-module cell processing/editing instrument 200. For example, a user may open and position each of the reagent cartridges 210 comprising various desired inserts and reagents within the chassis of the automated multi-module cell editing instrument 200 prior to activating cell processing. Further, each of the reagent cartridges 210 may be inserted into receptacles in the chassis having different temperature zones appropriate for the reagents contained therein.

Also illustrated in FIG. 2A is the robotic liquid handling system 258 including the gantry 202 and air displacement pipettor 232. In some examples, the robotic handling system 258 may include an automated liquid handling system such as those manufactured by Tecan Group Ltd. of Mannedorf, Switzerland, Hamilton Company of Reno, Nev. (see, e.g., WO2018015544A1), or Beckman Coulter, Inc. of Fort Collins, Colo. (see, e.g., US20160018427A1). Pipette tips may be provided in a pipette transfer tip supply (not shown) for use with the air displacement pipettor 232.

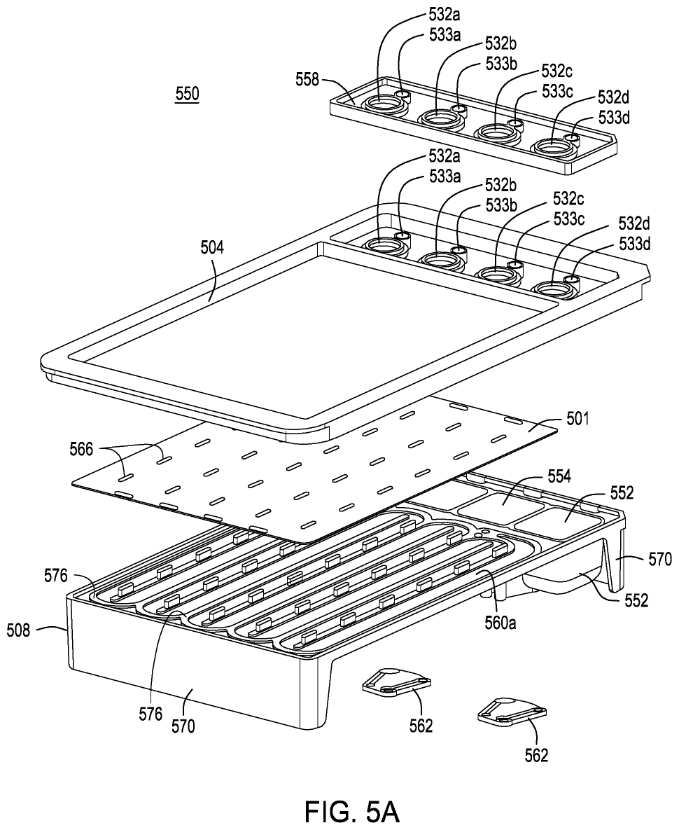

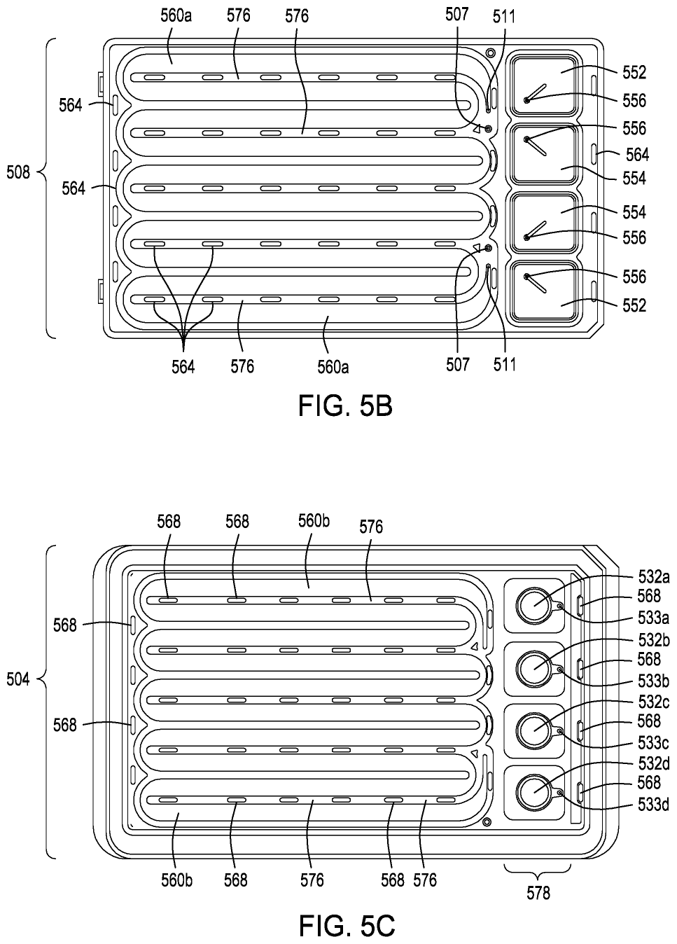

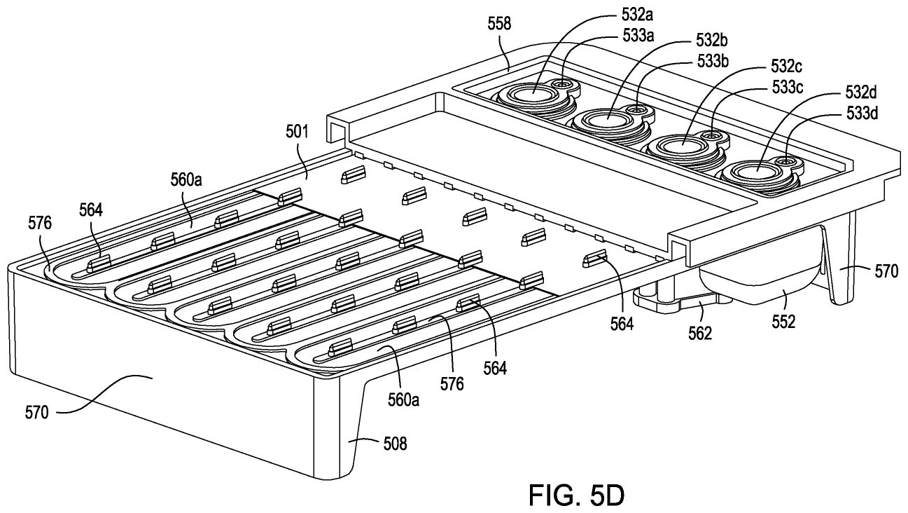

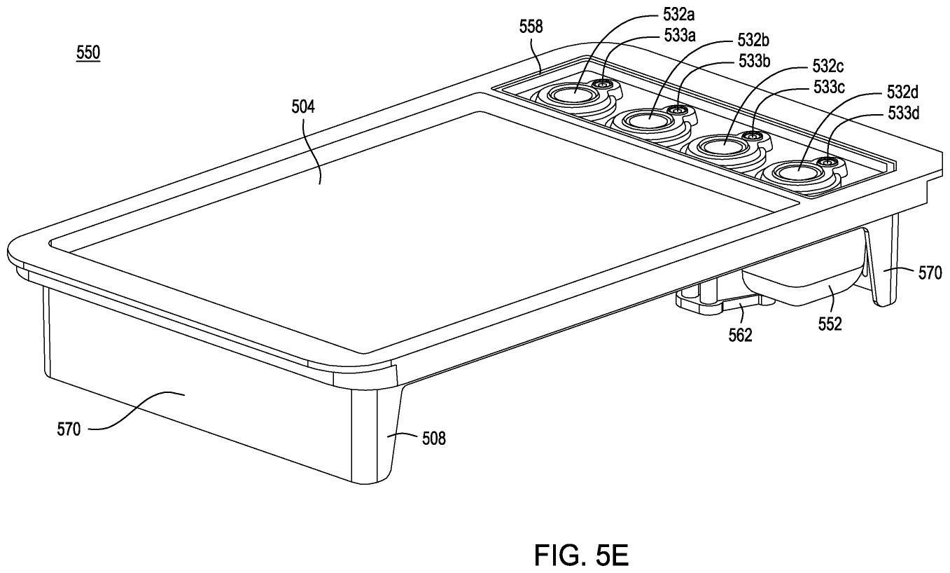

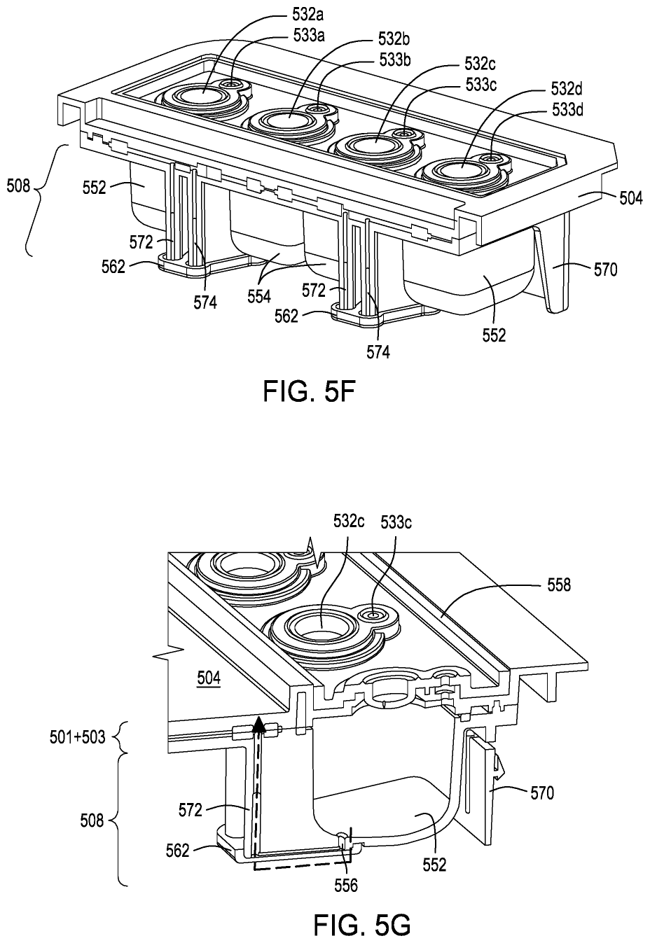

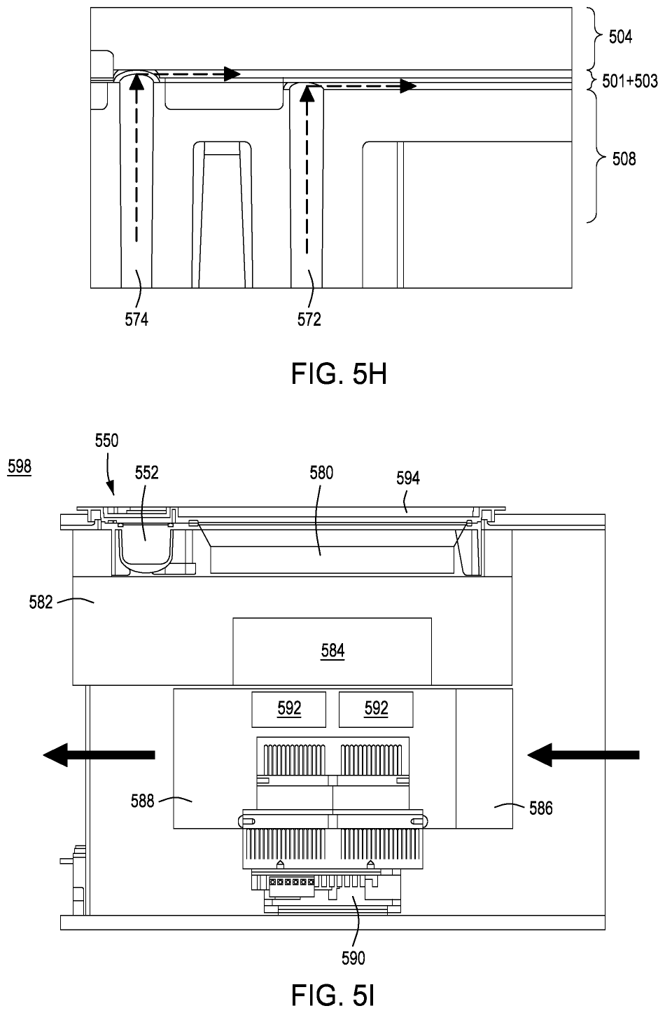

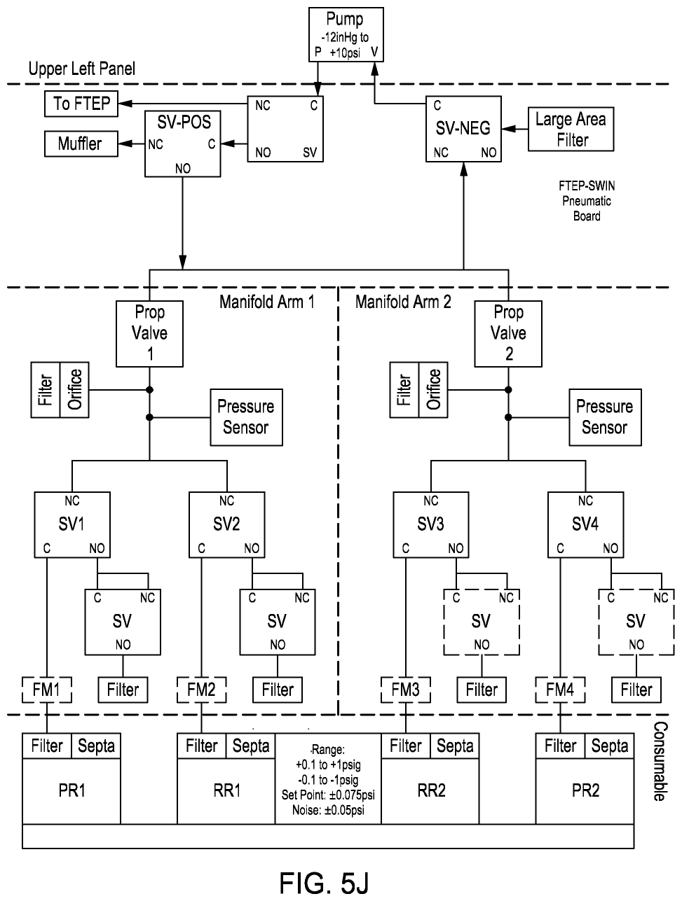

Inserts or components of the reagent cartridges 210, in some implementations, are marked with machine-readable indicia (not shown), such as bar codes, for recognition by the robotic handling system 258. For example, the robotic liquid handling system 258 may scan one or more inserts within each of the reagent cartridges 210 to confirm contents. In other implementations, machine-readable indicia may be marked upon each reagent cartridge 210, and a processing system (not shown, but see element 237 of FIG. 2B) of the automated multi-module cell editing instrument 200 may identify a stored materials map based upon the machine-readable indicia. In the embodiment illustrated in FIG. 2A, a cell growth module comprises two cell growth vials 218, 220 (described in greater detail below in relation to FIGS. 4A-4D). Additionally seen is the TFF module 222 (described above in detail in relation to FIGS. 1P-1EE). Also illustrated as part of the automated multi-module cell processing instrument 200 of FIG. 2A is a singulation module 240 (e.g., a solid wall isolation, incubation and normalization device (SWIIN device) is shown here) described herein in relation to FIGS. 5A-5J, served by, e.g., robotic liquid handing system 258 and air displacement pipettor 232. Also note the placement of three heatsinks 255.

FIG. 2B is a simplified representation of the contents of the exemplary multi-module cell processing instrument 200 depicted in FIG. 2A. Cartridge-based source materials (such as in reagent cartridges 210), for example, may be positioned in designated areas on a deck of the instrument 200 for access by an air displacement pipettor 232. The deck of the multi-module cell processing instrument 200 may include a protection sink such that contaminants spilling, dripping, or overflowing from any of the modules of the instrument 200 are contained within a lip of the protection sink. Also seen are reagent cartridges 210, which are shown disposed with thermal assemblies 211 which can create temperature zones appropriate for different regions. Note that one of the reagent cartridges also comprises a flow-through electroporation device 230 (FTEP), served by FTEP interface (e.g., manifold arm) and actuator 231. Also seen is TFF module 222 with adjacent thermal assembly 225, where the TFF module is served by TFF interface (e.g., manifold arm) and actuator 233. Thermal assemblies 225, 235, and 245 encompass thermal electric devices such as Peltier devices, as well as heatsinks, fans and coolers. The two rotating growth vials 218 and 220 are within a growth module 234, where the growth module is served by two thermal assemblies 235. Also seen is the SWIIN module 240, comprising a SWIIN cartridge 241, where the SWIIN module also comprises a thermal assembly 245, illumination 243 (in this embodiment, backlighting), evaporation and condensation control 249, and where the SWIIN module is served by SWIIN interface (e.g., manifold arm) and actuator 247. Also seen in this view is touch screen display 201, display actuator 203, illumination 205 (one on either side of multi-module cell processing instrument 200), and cameras 239 (one illumination device on either side of multi-module cell processing instrument 200). Finally, element 237 comprises electronics, such as circuit control boards, high-voltage amplifiers, power supplies, and power entry; as well as pneumatics, such as pumps, valves and sensors.

FIGS. 2C through 2E illustrate front perspective (door open), side perspective, and front perspective (door closed) views, respectively, of multi-module cell processing instrument 200 for use in as a desktop version of the automated multi-module cell editing instrument 200. For example, a chassis 290 may have a width of about 24-48 inches, a height of about 24-48 inches and a depth of about 24-48 inches. Chassis 290 may be and preferably is designed to hold all modules and disposable supplies used in automated cell processing and to perform all processes required without human intervention; that is, chassis 290 is configured to provide an integrated, stand-alone automated multi-module cell processing instrument. As illustrated in FIG. 2C, chassis 290 includes touch screen display 201, cooling grate 264, which allows for air flow via an internal fan (not shown). The touch screen display provides information to a user regarding the processing status of the automated multi-module cell editing instrument 200 and accepts inputs from the user for conducting the cell processing. In this embodiment, the chassis 290 is lifted by adjustable feet 270a, 270b, 270c and 270d (feet 270a-270c are shown in this FIG. 2C). Adjustable feet 270a-270d, for example, allow for additional air flow beneath the chassis 290.

Inside the chassis 290, in some implementations, will be most or all of the components described in relation to FIGS. 2A and 2B, including the robotic liquid handling system disposed along a gantry, reagent cartridges 210 including a flow-through electroporation device, one or more rotating growth vials 218, 220 in a cell growth module 234, a tangential flow filtration module 222, a SWIIN module 240 as well as interfaces and actuators for the various modules. In addition, chassis 290 houses control circuitry, liquid handling tubes, air pump controls, valves, sensors, thermal assemblies (e.g., heating and cooling units) and other control mechanisms. FIG. 2C is a side perspective view of automated multi-module cell editing instrument 200, showing chassis 290, touch screen display 201, adjustable feel 270b, 270c, and 270d, and cooling grates 264. FIG. 2D is a front perspective view of automated multi-module cell editing instrument 200 with the touch screen display (e.g., front door) 201 closed. Again seen are chassis 290, cooling grate 264, and adjustable feet 270a, 270b and 270c. For examples of multi-module cell editing instruments, see U.S. Pat. No. 10,253,316, issued 9 Apr. 2019; U.S. Pat. No. 10,329,559, issued 25 Jun. 2019; and U.S. Pat. No. 10,323,242, issued 18 Jun. 2019; and U.S. Ser. No. 16/412,175, filed 14 May 2019; Ser. No. 16/412,195, filed 14 May 2019; and Ser. No. 16/423,289, filed 28 May 2019, all of which are herein incorporated by reference in their entirety.

FIG. 3A depicts an exemplary combination reagent cartridge and electroporation device 300 (“cartridge”) that may be used in an automated multi-module cell processing instrument along with the TFF module. In addition, in certain embodiments the material used to fabricate the cartridge is thermally-conductive, as in certain embodiments the cartridge 300 contacts a thermal device (not shown), such as a Peltier device or thermoelectric cooler, that heats or cools reagents in the reagent reservoirs or reservoirs 304. Reagent reservoirs or reservoirs 304 may be reservoirs into which individual tubes of reagents are inserted as shown in FIG. 3A, or the reagent reservoirs may hold the reagents without inserted tubes. Additionally, the reservoirs in a reagent cartridge may be configured for any combination of tubes, co-joined tubes, and direct-fill of reagents.

In one embodiment, the reagent reservoirs or reservoirs 304 of reagent cartridge 300 are configured to hold various size tubes, including, e.g., 250 ml tubes, 25 ml tubes, 10 ml tubes, 5 ml tubes, and Eppendorf or microcentrifuge tubes. In yet another embodiment, all reservoirs may be configured to hold the same size tube, e.g., 5 ml tubes, and reservoir inserts may be used to accommodate smaller tubes in the reagent reservoir. In yet another embodiment—particularly in an embodiment where the reagent cartridge is disposable—the reagent reservoirs hold reagents without inserted tubes. In this disposable embodiment, the reagent cartridge may be part of a kit, where the reagent cartridge is pre-filled with reagents and the receptacles or reservoirs sealed with, e.g., foil, heat seal acrylic or the like and presented to a consumer where the reagent cartridge can then be used in an automated multi-module cell processing instrument. As one of ordinary skill in the art will appreciate given the present disclosure, the reagents contained in the reagent cartridge will vary depending on work flow; that is, the reagents will vary depending on the processes to which the cells are subjected in the automated multi-module cell processing instrument, e.g., protein production, cell transformation and culture, cell editing, etc.

Reagents such as cell samples, enzymes, buffers, nucleic acid vectors, expression cassettes, proteins or peptides, reaction components (such as, e.g., MgCl₂, dNTPs, nucleic acid assembly reagents, gap repair reagents, and the like), wash solutions, ethanol, and magnetic beads for nucleic acid purification and isolation, etc. may be positioned in the reagent cartridge at a known position. In some embodiments of cartridge 300, the cartridge comprises a script (not shown) readable by a processor (not shown) for dispensing the reagents. Also, the cartridge 300 as one component in an automated multi-module cell processing instrument may comprise a script specifying two, three, four, five, ten or more processes to be performed by the automated multi-module cell processing instrument. In certain embodiments, the reagent cartridge is disposable and is pre-packaged with reagents tailored to performing specific cell processing protocols, e.g., genome editing or protein production. Because the reagent cartridge contents vary while components/modules of the automated multi-module cell processing instrument or system may not, the script associated with a particular reagent cartridge matches the reagents used and cell processes performed. Thus, e.g., reagent cartridges may be pre-packaged with reagents for genome editing and a script that specifies the process steps for performing genome editing in an automated multi-module cell processing instrument, or, e.g., reagents for protein expression and a script that specifies the process steps for performing protein expression in an automated multi-module cell processing instrument.

For example, the reagent cartridge may comprise a script to pipette competent cells from a reservoir, transfer the cells to a transformation module, pipette a nucleic acid solution comprising a vector with expression cassette from another reservoir in the reagent cartridge, transfer the nucleic acid solution to the transformation module, initiate the transformation process for a specified time, then move the transformed cells to yet another reservoir in the reagent cassette or to another module such as a cell growth module in the automated multi-module cell processing instrument. In another example, the reagent cartridge may comprise a script to transfer a nucleic acid solution comprising a vector from a reservoir in the reagent cassette, nucleic acid solution comprising editing oligonucleotide cassettes in a reservoir in the reagent cassette, and a nucleic acid assembly mix from another reservoir to the nucleic acid assembly/desalting module, if present. The script may also specify process steps performed by other modules in the automated multi-module cell processing instrument. For example, the script may specify that the nucleic acid assembly/desalting reservoir be heated to 50° C. for 30 min to generate an assembled product; and desalting and resuspension of the assembled product via magnetic bead-based nucleic acid purification involving a series of pipette transfers and mixing of magnetic beads, ethanol wash, and buffer.

As described in relation to FIGS. 3B and 3C below, the exemplary reagent cartridges for use in the automated multi-module cell processing instruments may include one or more electroporation devices, preferably flow-through electroporation (FTEP) devices. In yet other embodiments, the reagent cartridge is separate from the transformation module. Electroporation is a widely-used method for permeabilization of cell membranes that works by temporarily generating pores in the cell membranes with electrical stimulation. Applications of electroporation include the delivery of DNA, RNA, siRNA, peptides, proteins, antibodies, drugs or other substances to a variety of cells such as mammalian cells (including human cells), plant cells, archea, yeasts, other eukaryotic cells, bacteria, and other cell types. Electrical stimulation may also be used for cell fusion in the production of hybridomas or other fused cells. During a typical electroporation procedure, cells are suspended in a buffer or medium that is favorable for cell survival. For bacterial cell electroporation, low conductance mediums, such as water, glycerol solutions and the like, are often used to reduce the heat production by transient high current. In traditional electroporation devices, the cells and material to be electroporated into the cells (collectively “the cell sample”) are placed in a cuvette embedded with two flat electrodes for electrical discharge. For example, Bio-Rad (Hercules, Calif.) makes the GENE PULSER XCELL™ line of products to electroporate cells in cuvettes. Traditionally, electroporation requires high field strength; however, the flow-through electroporation devices included in the reagent cartridges achieve high efficiency cell electroporation with low toxicity. The reagent cartridges of the disclosure allow for particularly easy integration with robotic liquid handling instrumentation that is typically used in automated instruments and systems such as air displacement pipettors. Such automated instrumentation includes, but is not limited to, off-the-shelf automated liquid handling systems from Tecan (Mannedorf, Switzerland), Hamilton (Reno, Nev.), Beckman Coulter (Fort Collins, Colo.), etc.

FIGS. 3B and 3C are top perspective and bottom perspective views, respectively, of an exemplary FTEP device 350 that may be part of (e.g., a component in) reagent cartridge 300 in FIG. 3A or may be a stand-alone module; that is, not a part of a reagent cartridge or other module. FIG. 3B depicts an FTEP device 350. The FTEP device 350 has wells that define cell sample inlets 352 and cell sample outlets 354. FIG. 3C is a bottom perspective view of the FTEP device 350 of FIG. 3B. An inlet well 352 and an outlet well 354 can be seen in this view. Also seen in FIG. 3C are the bottom of an inlet 362 corresponding to well 352, the bottom of an outlet 364 corresponding to the outlet well 354, the bottom of a defined flow channel 366 and the bottom of two electrodes 368 on either side of flow channel 366. The FTEP devices may comprise push-pull pneumatic means to allow multi-pass electroporation procedures; that is, cells to electroporated may be “pulled” from the inlet toward the outlet for one pass of electroporation, then be “pushed” from the outlet end of the FTEP device toward the inlet end to pass between the electrodes again for another pass of electroporation. Further, this process may be repeated one to many times. For additional information regarding FTEP devices, see, e.g., U.S. Ser. No. 16/147,120, filed 28 Sep. 2018; Ser. No. 16/147,353, filed 28 Sep. 2019; Ser. No. 16/426,310, filed 30 May 2019; and Ser. No. 16/147,871, filed 30 Sep. 2018; and U.S. Pat. No. 10,323,258, issued 18 Jun. 2019. Further, other embodiments of the reagent cartridge may provide or accommodate electroporation devices that are not configured as FTEP devices, such as those described in U.S. Ser. No. 16/109,156, filed 22 Aug. 2018. For reagent cartridges useful in the present automated multi-module cell processing instruments, see, e.g., U.S. Pat. No. 10,376,889, issued 13 Aug. 2019; and U.S. Ser. No. 16,451,601, filed 25 Jun. 2019.

Additional details of the FTEP devices are illustrated in FIGS. 3D-3M. Note that in the FTEP devices in FIGS. 3D-3M the electrodes are placed such that a first electrode is placed between an inlet and a narrowed region of the flow channel, and the second electrode is placed between the narrowed region of the flow channel and an outlet. FIG. 3D shows a top planar view of an FTEP device 350 having an inlet 352 for introducing a fluid containing cells and exogenous material into FTEP device 350 and an outlet 354 for removing the transformed cells from the FTEP following electroporation. The electrodes 368 are introduced through channels (not shown) in the device. FIG. 3E shows a cutaway view from the top of the FTEP device 350, with the inlet 352, outlet 354, and electrodes 368 positioned with respect to a flow channel 366. FIG. 3F shows a side cutaway view of FTEP device 350 with the inlet 352 and inlet channel 372, and outlet 354 and outlet channel 374. The electrodes 368 are positioned in electrode channels 376 so that they are in fluid communication with the flow channel 366, but not directly in the path of the cells traveling through the flow channel 366. Note that the first electrode is placed between the inlet and the narrowed region of the flow channel, and the second electrode is placed between the narrowed region of the flow channel and the outlet. The electrodes 368 in this aspect of the device are positioned in the electrode channels 376 which are generally perpendicular to the flow channel 366 such that the fluid containing the cells and exogenous material flows from the inlet channel 372 through the flow channel 366 to the outlet channel 374, and in the process fluid flows into the electrode channels 376 to be in contact with the electrodes 368. In this aspect, the inlet channel, outlet channel and electrode channels all originate from the same planar side of the device. In certain aspects, however, the electrodes may be introduced from a different planar side of the FTEP device than the inlet and outlet channels.

In the FTEP devices of the disclosure, the toxicity level of the transformation results in greater than 30% viable cells after electroporation, preferably greater than 35%, 40%, 45%, 50%, 55%, 60%, 70%, 75%, 80%, 85%, 90%, 95% or even 99% viable cells following transformation, depending on the cell type and the nucleic acids being introduced into the cells.

The housing of the FTEP device can be made from many materials depending on whether the FTEP device is to be reused, autoclaved, or is disposable, including stainless steel, silicon, glass, resin, polyvinyl chloride, polyethylene, polyamide, polystyrene, polyethylene, polypropylene, acrylonitrile butadiene, polycarbonate, polyetheretheketone (PEEK), polysulfone and polyurethane, co-polymers of these and other polymers. Similarly, the walls of the channels in the device can be made of any suitable material including silicone, resin, glass, glass fiber, polyvinyl chloride, polyethylene, polyamide, polyethylene, polypropylene, acrylonitrile butadiene, polycarbonate, polyetheretheketone (PEEK), polysulfone and polyurethane, co-polymers of these and other polymers. Preferred materials include crystal styrene, cyclo-olefin polymer (COP) and cyclic olephin co-polymers (COC), which allow the device to be formed entirely by injection molding in one piece with the exception of the electrodes and, e.g., a bottom sealing film if present.

The FTEP devices described herein (or portions of the FTEP devices) can be created or fabricated via various techniques, e.g., as entire devices or by creation of structural layers that are fused or otherwise coupled. For example, for metal FTEP devices, fabrication may include precision mechanical machining or laser machining; for silicon FTEP devices, fabrication may include dry or wet etching; for glass FTEP devices, fabrication may include dry or wet etching, powderblasting, sandblasting, or photostructuring; and for plastic FTEP devices fabrication may include thermoforming, injection molding, hot embossing, or laser machining. The components of the FTEP devices may be manufactured separately and then assembled, or certain components of the FTEP devices (or even the entire FTEP device except for the electrodes) may be manufactured (e.g., using 3D printing) or molded (e.g., using injection molding) as a single entity, with other components added after molding. For example, housing and channels may be manufactured or molded as a single entity, with the electrodes later added to form the FTEP unit. Alternatively, the FTEP device may also be formed in two or more parallel layers, e.g., a layer with the horizontal channel and filter, a layer with the vertical channels, and a layer with the inlet and outlet ports, which are manufactured and/or molded individually and assembled following manufacture.

In specific aspects, the FTEP device can be manufactured using a circuit board as a base, with the electrodes, filter and/or the flow channel formed in the desired configuration on the circuit board, and the remaining housing of the device containing, e.g., the one or more inlet and outlet channels and/or the flow channel formed as a separate layer that is then sealed onto the circuit board. The sealing of the top of the housing onto the circuit board provides the desired configuration of the different elements of the FTEP devices of the disclosure. Also, two to many FTEP devices may be manufactured on a single substrate, then separated from one another thereafter or used in parallel. In certain embodiments, the FTEP devices are reusable and, in some embodiments, the FTEP devices are disposable. In additional embodiments, the FTEP devices may be autoclavable.

The electrodes 408 can be formed from any suitable metal, such as copper, stainless steel, titanium, aluminum, brass, silver, rhodium, gold or platinum, or graphite. One preferred electrode material is alloy 303 (UNS330300) austenitic stainless steel. An applied electric field can destroy electrodes made from of metals like aluminum. If a multiple-use (i.e., non-disposable) flow-through FTEP device is desired—as opposed to a disposable, one-use flow-through FTEP device—the electrode plates can be coated with metals resistant to electrochemical corrosion. Conductive coatings like noble metals, e.g., gold, can be used to protect the electrode plates.

As mentioned, the FTEP devices may comprise push-pull pneumatic means to allow multi-pass electroporation procedures; that is, cells to electroporated may be “pulled” from the inlet toward the outlet for one pass of electroporation, then be “pushed” from the outlet end of the flow-through FTEP device toward the inlet end to pass between the electrodes again for another pass of electroporation. This process may be repeated one to many times.

Depending on the type of cells to be electroporated (e.g., bacterial, yeast, mammalian) and the configuration of the electrodes, the distance between the electrodes in the flow channel can vary widely. For example, where the flow channel decreases in width, the flow channel may narrow to between 10 μm and 5 mm, or between 25 μm and 3 mm, or between 50 μm and 2 mm, or between 75 μm and 1 mm. The distance between the electrodes in the flow channel may be between 1 mm and 10 mm, or between 2 mm and 8 mm, or between 3 mm and 7 mm, or between 4 mm and 6 mm. The overall size of the FTEP device may be from 3 cm to 15 cm in length, or 4 cm to 12 cm in length, or 4.5 cm to 10 cm in length. The overall width of the FTEP device may be from 0.5 cm to 5 cm, or from 0.75 cm to 3 cm, or from 1 cm to 2.5 cm, or from 1 cm to 1.5 cm.

The region of the flow channel that is narrowed is wide enough so that at least two cells can fit in the narrowed portion side-by-side. For example, a typical bacterial cell is 1 μm in diameter; thus, the narrowed portion of the flow channel of the FTEP device used to transform such bacterial cells will be at least 2 μm wide. In another example, if a mammalian cell is approximately 50 μm in diameter, the narrowed portion of the flow channel of the FTEP device used to transform such mammalian cells will be at least 100 μm wide. That is, the narrowed portion of the FTEP device will not physically contort or “squeeze” the cells being transformed.

In embodiments of the FTEP device where reservoirs are used to introduce cells and exogenous material into the FTEP device, the reservoirs range in volume from 100 μL to 10 mL, or from 500 μL to 75 mL, or from 1 mL to 5 mL. The flow rate in the FTEP ranges from 0.1 mL to 5 mL per minute, or from 0.5 mL to 3 mL per minute, or from 1.0 mL to 2.5 mL per minute. The pressure in the FTEP device ranges from 1-30 psi, or from 2-10 psi, or from 3-5 psi.

To avoid different field intensities between the electrodes, the electrodes should be arranged in parallel. Furthermore, the surface of the electrodes should be as smooth as possible without pin holes or peaks. Electrodes having a roughness Rz of 1 to 10 μm are preferred. In another embodiment of the invention, the flow-through electroporation device comprises at least one additional electrode which applies a ground potential to the FTEP device.

FIGS. 3G-3I illustrate an alternative embodiment of the FTEP devices of the disclosure. FIG. 3G shows a top planar view of an FTEP device 380 having a first inlet 352 for introducing a fluid containing cells into FTEP device 380 and an outlet 354 for removing the transformed cells from the FTEP device 380 following electroporation. However, in this FTEP device, there is a second inlet 356 for introducing exogenous material to be electroporated to the cells. The electrodes 368 are introduced through channels (not shown). FIG. 3H shows a cutaway view from the top of the FTEP device 380, with the first inlet 352, second inlet 356, outlet 354, and the electrodes 368 positioned with respect to the flow channel 366. FIG. 3I shows a side cutaway view of FTEP device 380 with inlets 352, 356 and inlet channels 372, 378 and outlet 354 and outlet channel 374. The electrodes 368 are positioned in the electrode channels 376 so that they are in fluid communication with the flow channel 366, but not substantially in the path of the cells traveling through the flow channel 366. The electrodes 368 in this aspect of the FTEP device 380 are positioned in the electrode channels 376 where the electrode channels 376 are generally perpendicular to the flow channel 366 such that fluid containing the cells and fluid containing the exogenous materials flow from the inlets 352, 356 through the inlet channels 372, 378 into the flow channel 366 and through to the outlet channel 374, and in the process the cells and exogenous material in medium flow into the electrode channels 376 to be in contact with the electrodes 368. One of the two electrodes 368 and electrode channels 376 is positioned between inlets 352 and 356 and inlet channels 372 and 378 and the narrowed region (not shown) of flow channel 366, and the other electrode 368 and electrode channel 376 is positioned between the narrowed region (not shown) of flow channel 366 and the outlet channel 374 and outlet 354. In FIG. 3I, the inlet channel, outlet channel and electrode channels all originate from the same planar side of the device, although the electrodes (and inlets and outlet) can also be configured to originate from different sides of the FTEP device.

FIGS. 3J-3M illustrate another alternative embodiment of the devices of the disclosure. FIG. 3J shows a top planar view of an electroporation device 390 having an inlet 352 for introducing a fluid containing cells and exogenous material into the FTEP device 390 and an outlet 354 for removal of the transformed cells from the FTEP device 390 following electroporation. The electrodes 368 are introduced through channels (not shown) machined into the device. FIG. 3K shows a cutaway view from the top of the device 390, showing an inlet 352, an outlet 354, a filter 392 of substantially uniform density, and electrodes 368 positioned with respect to the flow channel 366. FIG. 3L shows a cutaway view from the top of an alternative configuration 395 of the FTEP device, with an inlet 352, an outlet 354, a filter 394 of increasing gradient density, and electrodes 368 positioned with respect to the flow channel 366. In FIGS. 3J-3L, like FIGS. 3G-3I, the first electrode is placed between the inlet and the narrowed region of the flow channel, and the second electrode is placed between the narrowed region of the flow channel and the outlet. In some embodiments such as those depicted in FIGS. 3J-3L, the FTEP devices comprise a filter disposed within the flow channel after the inlet channel and before the first electrode channel. The filter may be substantially homogeneous in porosity (e.g., have a uniform density as in FIG. 3K); alternatively, the filter may increase in gradient density where the end of the filter proximal to the inlet is less dense, and the end of the filter proximal to the outlet is more dense (as shown in FIG. 3L). The filter may be fashioned from any suitable and preferably inexpensive material, including porous plastics, hydrophobic polyethylene, cotton, glass fibers, or the filter may be integral with and fabricated as part of the FTEP device body.

FIG. 3M shows a side cutaway view of an FTEP device 397 with an inlet 352 and an inlet channel 372, and an outlet 354 and an outlet channel 374. The electrodes 368 are positioned in the electrode channels 376 so that they are in fluid communication with the flow channel 366, but not directly in the path of the cells traveling through flow channel 366. Note that filter 392/394 is positioned between inlet 352 and inlet channel 372 and electrodes 368 and electrode channels 376. The electrodes 368 are positioned in the electrode channels 376 and perpendicular to the flow channel 366 such that fluid containing the cells and exogenous material flows from the inlet channel 352 through the flow channel 366 to the outlet channel 374, and in the process fluid flows into the electrode channels 376 to be in contact with both electrodes 368.

FIG. 4A shows one embodiment of a rotating growth vial 400 for use with the cell growth device described herein. The rotating growth vial 400 is an optically-transparent container having an open end 404 for receiving liquid media and cells, a central vial region 406 that defines the primary container for growing cells, a tapered-to-constricted region 418 defining at least one light path 410, a closed end 416, and a drive engagement mechanism 412. The rotating growth vial 400 has a central longitudinal axis 420 around which the vial rotates, and the light path 410 is generally perpendicular to the longitudinal axis of the vial. The first light path 410 is positioned in the lower constricted portion of the tapered-to-constricted region 418. Optionally, some embodiments of the rotating growth vial 400 have a second light path 408 in the tapered region of the tapered-to-constricted region 418. Both light paths in this embodiment are positioned in a region of the rotating growth vial that is constantly filled with the cell culture (cells+growth media) and are not affected by the rotational speed of the growth vial. The first light path 410 is shorter than the second light path 408 allowing for sensitive measurement of OD values when the OD values of the cell culture in the vial are at a high level (e.g., later in the cell growth process), whereas the second light path 408 allows for sensitive measurement of OD values when the OD values of the cell culture in the vial are at a lower level (e.g., earlier in the cell growth process).

The drive engagement mechanism 412 engages with a motor (not shown) to rotate the vial. In some embodiments, the motor drives the drive engagement mechanism 412 such that the rotating growth vial 400 is rotated in one direction only, and in other embodiments, the rotating growth vial 400 is rotated in a first direction for a first amount of time or periodicity, rotated in a second direction (i.e., the opposite direction) for a second amount of time or periodicity, and this process may be repeated so that the rotating growth vial 400 (and the cell culture contents) are subjected to an oscillating motion. Further, the choice of whether the culture is subjected to oscillation and the periodicity therefor may be selected by the user. The first amount of time and the second amount of time may be the same or may be different. The amount of time may be 1, 2, 3, 4, 5, or more seconds, or may be 1, 2, 3, 4 or more minutes. In another embodiment, in an early stage of cell growth the rotating growth vial 400 may be oscillated at a first periodicity (e.g., every 60 seconds), and then a later stage of cell growth the rotating growth vial 400 may be oscillated at a second periodicity (e.g., every one second) different from the first periodicity.

The rotating growth vial 400 may be reusable or, preferably, the rotating growth vial is consumable. In some embodiments, the rotating growth vial is consumable and is presented to the user pre-filled with growth medium, where the vial is hermetically sealed at the open end 404 with a foil seal. A medium-filled rotating growth vial packaged in such a manner may be part of a kit for use with a stand-alone cell growth device or with a cell growth module that is part of an automated multi-module cell processing system. To introduce cells into the vial, a user need only pipette up a desired volume of cells and use the pipette tip to punch through the foil seal of the vial. Open end 404 may optionally include an extended lip 402 to overlap and engage with the cell growth device. In automated systems, the rotating growth vial 400 may be tagged with a barcode or other identifying means that can be read by a scanner or camera (not shown) that is part of the automated system.

The volume of the rotating growth vial 400 and the volume of the cell culture (including growth medium) may vary greatly, but the volume of the rotating growth vial 400 must be large enough to generate a specified total number of cells. In practice, the volume of the rotating growth vial 400 may range from 1-250 mL, 2-100 mL, from 5-80 mL, 10-50 mL, or from 12-35 mL. Likewise, the volume of the cell culture (cells+growth media) should be appropriate to allow proper aeration and mixing in the rotating growth vial 400. Proper aeration promotes uniform cellular respiration within the growth media. Thus, the volume of the cell culture should be approximately 5-85% of the volume of the growth vial or from 20-60% of the volume of the growth vial. For example, for a 30 mL growth vial, the volume of the cell culture would be from about 1.5 mL to about 26 mL, or from 6 mL to about 18 mL.

The rotating growth vial 400 preferably is fabricated from a bio-compatible optically transparent material—or at least the portion of the vial comprising the light path(s) is transparent. Additionally, material from which the rotating growth vial is fabricated should be able to be cooled to about 4° C. or lower and heated to about 55° C. or higher to accommodate both temperature-based cell assays and long-term storage at low temperatures. Further, the material that is used to fabricate the vial must be able to withstand temperatures up to 55° C. without deformation while spinning. Suitable materials include cyclic olefin copolymer (COC), glass, polyvinyl chloride, polyethylene, polyamide, polypropylene, polycarbonate, poly(methyl methacrylate (PMMA), polysulfone, polyurethane, and co-polymers of these and other polymers. Preferred materials include polypropylene, polycarbonate, or polystyrene. In some embodiments, the rotating growth vial is inexpensively fabricated by, e.g., injection molding or extrusion.

FIG. 4B is a perspective view of one embodiment of a cell growth device 430. FIG. 4C depicts a cut-away view of the cell growth device 430 from FIG. 4B. In both figures, the rotating growth vial 400 is seen positioned inside a main housing 436 with the extended lip 402 of the rotating growth vial 400 extending above the main housing 436. Additionally, end housings 452, a lower housing 432 and flanges 434 are indicated in both figures. Flanges 434 are used to attach the cell growth device 430 to heating/cooling means or other structure (not shown). FIG. 4C depicts additional detail. In FIG. 4C, upper bearing 442 and lower bearing 440 are shown positioned within main housing 436. Upper bearing 442 and lower bearing 440 support the vertical load of rotating growth vial 400. Lower housing 432 contains the drive motor 438. The cell growth device 430 of FIG. 4C comprises two light paths: a primary light path 444, and a secondary light path 450. Light path 444 corresponds to light path 410 positioned in the constricted portion of the tapered-to-constricted portion of the rotating growth vial 400, and light path 450 corresponds to light path 408 in the tapered portion of the tapered-to-constricted portion of the rotating growth via 416. Light paths 410 and 408 are not shown in FIG. 4C but may be seen in FIG. 4A. In addition to light paths 444 and 440, there is an emission board 448 to illuminate the light path(s), and detector board 446 to detect the light after the light travels through the cell culture liquid in the rotating growth vial 400.

The motor 438 engages with drive mechanism 412 and is used to rotate the rotating growth vial 400. In some embodiments, motor 438 is a brushless DC type drive motor with built-in drive controls that can be set to hold a constant revolution per minute (RPM) between 0 and about 3000 RPM. Alternatively, other motor types such as a stepper, servo, brushed DC, and the like can be used. Optionally, the motor 438 may also have direction control to allow reversing of the rotational direction, and a tachometer to sense and report actual RPM. The motor is controlled by a processor (not shown) according to, e.g., standard protocols programmed into the processor and/or user input, and the motor may be configured to vary RPM to cause axial precession of the cell culture thereby enhancing mixing, e.g., to prevent cell aggregation, increase aeration, and optimize cellular respiration.

Main housing 436, end housings 452 and lower housing 432 of the cell growth device 430 may be fabricated from any suitable, robust material including aluminum, stainless steel, and other thermally conductive materials, including plastics. These structures or portions thereof can be created through various techniques, e.g., metal fabrication, injection molding, creation of structural layers that are fused, etc. Whereas the rotating growth vial 400 is envisioned in some embodiments to be reusable, but preferably is consumable, the other components of the cell growth device 430 are preferably reusable and function as a stand-alone benchtop device or as a module in a multi-module cell processing system.

The processor (not shown) of the cell growth device 430 may be programmed with information to be used as a “blank” or control for the growing cell culture. A “blank” or control is a vessel containing cell growth medium only, which yields 100% transmittance and 0 OD, while the cell sample will deflect light rays and will have a lower percent transmittance and higher OD. As the cells grow in the media and become denser, transmittance will decrease and OD will increase. The processor (not shown) of the cell growth device 430 may be programmed to use wavelength values for blanks commensurate with the growth media typically used in cell culture (whether, e.g., mammalian cells, bacterial cells, animal cells, yeast cells, etc.). Alternatively, a second spectrophotometer and vessel may be included in the cell growth device 430, where the second spectrophotometer is used to read a blank at designated intervals.

FIG. 4D illustrates a cell growth device 430 as part of an assembly comprising the cell growth device 430 of FIG. 4B coupled to light source 490, detector 492, and thermal components 494. The rotating growth vial 400 is inserted into the cell growth device. Components of the light source 490 and detector 492 (e.g., such as a photodiode with gain control to cover 5-log) are coupled to the main housing of the cell growth device. The lower housing 432 that houses the motor that rotates the rotating growth vial 400 is illustrated, as is one of the flanges 434 that secures the cell growth device 430 to the assembly. Also, the thermal components 494 illustrated are a Peltier device or thermoelectric cooler. In this embodiment, thermal control is accomplished by attachment and electrical integration of the cell growth device 430 to the thermal components 494 via the flange 434 on the base of the lower housing 432. Thermoelectric coolers are capable of “pumping” heat to either side of a junction, either cooling a surface or heating a surface depending on the direction of current flow. In one embodiment, a thermistor is used to measure the temperature of the main housing and then, through a standard electronic proportional-integral-derivative (PID) controller loop, the rotating growth vial 400 is controlled to approximately +/−0.5° C.

In use, cells are inoculated (cells can be pipetted, e.g., from an automated liquid handling system or by a user) into pre-filled growth media of a rotating growth vial 400 by piercing though the foil seal or film. The programmed software of the cell growth device 430 sets the control temperature for growth, typically 30° C., then slowly starts the rotation of the rotating growth vial 400. The cell/growth media mixture slowly moves vertically up the wall due to centrifugal force allowing the rotating growth vial 400 to expose a large surface area of the mixture to a normal oxygen environment. The growth monitoring system takes either continuous readings of the OD or OD measurements at pre-set or pre-programmed time intervals. These measurements are stored in internal memory and if requested the software plots the measurements versus time to display a growth curve. If enhanced mixing is required, e.g., to optimize growth conditions, the speed of the vial rotation can be varied to cause an axial precession of the liquid, and/or a complete directional change can be performed at programmed intervals. The growth monitoring can be programmed to automatically terminate the growth stage at a pre-determined OD, and then quickly cool the mixture to a lower temperature to inhibit further growth.

One application for the cell growth device 430 is to constantly measure the optical density of a growing cell culture. One advantage of the described cell growth device is that optical density can be measured continuously (kinetic monitoring) or at specific time intervals; e.g., every 5, 10, 15, 20, 30 45, or 60 seconds, or every 1, 2, 3, 4, 5, 6, 7, 8, 9, or 10 minutes. While the cell growth device 430 has been described in the context of measuring the optical density (OD) of a growing cell culture, it should, however, be understood by a skilled artisan given the teachings of the present specification that other cell growth parameters can be measured in addition to or instead of cell culture OD. As with optional measure of cell growth in relation to the solid wall device or module described supra, spectroscopy using visible, UV, or near infrared (NIR) light allows monitoring the concentration of nutrients and/or wastes in the cell culture and other spectroscopic measurements may be made; that is, other spectral properties can be measured via, e.g., dielectric impedance spectroscopy, visible fluorescence, fluorescence polarization, or luminescence. Additionally, the cell growth device 430 may include additional sensors for measuring, e.g., dissolved oxygen, carbon dioxide, pH, conductivity, and the like.

Another module useful in multi-module cell processing is a solid wall isolation, incubation, and normalization (SWIIN) module. FIG. 5A depicts an embodiment of a SWIIN module 550 from an exploded top perspective view. The SWIIN module embodiment described in relation to FIGS. 5A-5J provides advantages over other singulation or isolation devices. For example, the positioning of the reservoirs and reservoir ports below the retentate and permeate serpentine channels minimizes instantaneous flow of fluid in the reservoirs through the reservoir ports and into channels that connect the reservoir ports to the retentate and permeate channels. Instead, flow is controlled by the application of pressure (positive or negative) and an appropriate time chosen by the user. In SWIIN module 550 the retentate member is formed on the bottom of a top of a SWIIN module component and the permeate member is formed on the top of the bottom of a SWIIN module component.

The SWIIN module 550 in FIG. 5A comprises from the top down, a reservoir gasket or cover 558, a retentate member 504 (where a retentate flow channel cannot be seen in this FIG. 5A), a perforated member 501 swaged with a filter (filter not seen in FIG. 5A), a permeate member 508 comprising integrated reservoirs (permeate reservoirs 552 and retentate reservoirs 554), and two reservoir seals 562, which seal the bottom of permeate reservoirs 552 and retentate reservoirs 554. A permeate channel 560a can be seen disposed on the top of permeate member 508, defined by a raised portion 576 of serpentine channel 560a, and ultrasonic tabs 564 can be seen disposed on the top of permeate member 508 as well. The perforations that form the wells on perforated member 501 are not seen in this FIG. 5A; however, through-holes 566 to accommodate the ultrasonic tabs 564 are seen. In addition, supports 570 are disposed at either end of SWIIN module 550 to support SWIIN module 550 and to elevate permeate member 508 and retentate member 504 above reservoirs 552 and 554 to minimize bubbles or air entering the fluid path from the permeate reservoir to serpentine channel 560a or the fluid path from the retentate reservoir to serpentine channel 560b (neither fluid path is seen in this FIG. 5A, but see FIG. 5H).

In this FIG. 5A, it can be seen that the serpentine channel 560a that is disposed on the top of permeate member 508 traverses permeate member 508 for most of the length of permeate member 508 except for the portion of permeate member 508 that comprises permeate reservoirs 552 and retentate reservoirs 554 and for most of the width of permeate member 508. As used herein with respect to the distribution channels in the retentate member or permeate member, “most of the length” means about 95% of the length of the retentate member or permeate member, or about 90%, 85%, 80%, 75%, or 70% of the length of the retentate member or permeate member. As used herein with respect to the distribution channels in the retentate member or permeate member, “most of the width” means about 95% of the width of the retentate member or permeate member, or about 90%, 85%, 80%, 75%, or 70% of the width of the retentate member or permeate member.

In this embodiment of a SWIIN module, the perforated member includes through-holes to accommodate ultrasonic tabs disposed on the permeate member. Thus, in this embodiment the perforated member is fabricated from 316 stainless steel, and the perforations form the walls of microwells while a filter or membrane is used to form the bottom of the microwells. Typically, the perforations (microwells) are approximately 150 μm-200 μm in diameter, and the perforated member is approximately 125 μm deep, resulting in microwells having a volume of approximately 2.5 nl, with a total of approximately 200,000 microwells. The distance between the microwells is approximately 279 μm center-to-center. Though here the microwells have a volume of approximately 2.5 nl, the volume of the microwells may be from 1 to 25 nl, or preferably from 2 to 10 nl, and even more preferably from 2 to 4 nl. As for the filter or membrane, like the filter described previously, filters appropriate for use are solvent resistant, contamination free during filtration, and are able to retain the types and sizes of cells of interest. For example, in order to retain small cell types such as bacterial cells, pore sizes can be as low as 0.10 μm, however for other cell types (e.g., such as for mammalian cells), the pore sizes can be as high as 10.0 μm-20.0 μm or more. Indeed, the pore sizes useful in the cell concentration device/module include filters with sizes from 0.10 μm, 0.11 μm, 0.12 μm, 0.13 μm, 0.14 μm, 0.15 μm, 0.16 μm, 0.17 μm, 0.18 μm, 0.19 μm, 0.20 μm, 0.21 μm, 0.22 μm, 0.23 μm, 0.24 μm, 0.25 μm, 0.26 μm, 0.27 μm, 0.28 μm, 0.29 μm, 0.30 μm, 0.31 μm, 0.32 μm, 0.33 μm, 0.34 μm, 0.35 μm, 0.36 μm, 0.37 μm, 0.38 μm, 0.39 μm, 0.40 μm, 0.41 μm, 0.42 μm, 0.43 μm, 0.44 μm, 0.45 μm, 0.46 μm, 0.47 μm, 0.48 μm, 0.49 μm, 0.50 μm and larger. The filters may be fabricated from any suitable material including cellulose mixed ester (cellulose nitrate and acetate) (CME), polycarbonate (PC), polyvinylidene fluoride (PVDF), polyethersulfone (PES), polytetrafluoroethylene (PTFE), nylon, or glass fiber.

FIG. 5B is a top-down view of permeate member 508, showing serpentine channel 560a (the portion of the serpentine channel disposed in permeate member 508) defined by raised portion 576 of serpentine channel 560a, permeate reservoirs 552, retentate reservoirs 554, reservoir ports 556 (two of the four of which are labeled), ultrasonic tabs 564 disposed at each end of permeate member 508 and on the raised portion 576 of serpentine channel 560a of permeate member 508, two permeate ports 511, and two retentate ports 507 are also seen.

FIG. 5C is a bottom-up view of retentate member 504, showing serpentine channel 560b (the portion of the serpentine channel disposed in retentate member 508) defined by the raised portion 576 of the serpentine channel 560b. Also seen is an integrated reservoir cover 578 for the permeate and retentate reservoirs that mate with permeate reservoirs 552 and retentate reservoirs 554 on the permeate member. The integrated reservoir cover 578 comprises reservoir access apertures 532a, 532b, 532c, and 532d, as well as pneumatic ports 533a, 533b, 533c and 533d. As with previous embodiments, the serpentine channel 560a of permeate member 508 and the serpentine channel 560b of retentate member 504 mate to form the top (retentate member) and bottom (permeate member) of a mated serpentine channel. The footprint length of the serpentine channel structure is from, e.g., from 80 mm to 500 mm, from 100 mm to 400 mm, or from 150 mm to 250 mm. In some aspects, the entire footprint width of the channel structure is from 50 mm to 200 mm, from 75 mm to 175 mm, or from 100 mm to 150 mm.

The cross-section configuration of the mated serpentine channel may be round, elliptical, oval, square, rectangular, trapezoidal, or irregular. If square, rectangular, or another shape with generally straight sides, the cross section may be from about 2 mm to 15 mm wide, or from 3 mm to 12 mm wide, or from 5 mm to 10 mm wide. If the cross section of the mated serpentine channel is generally round, oval or elliptical, the radius of the channel may be from about 3 mm to 20 mm in hydraulic radius, or from 5 mm to 15 mm in hydraulic radius, or from 8 mm to 12 mm in hydraulic radius.

As in previous embodiments, disposed between serpentine channels 560a and 560b is perforated member 501 (adjacent retentate member 504) and filter 503 (adjacent permeate member 508), where filter 503 is swaged with perforated member 501. Serpentine channels 560a and 560b can have approximately the same volume or a different volume. For example, each “side” or portion 560a, 560b of the serpentine channel may have a volume of, e.g., 2 mL, or serpentine channel 560a of permeate member 508 may have a volume of 2 mL, and the serpentine channel 560b of retentate member 504 may have a volume of, e.g., 3 mL. The volume of fluid in the serpentine channel may range from about 2 mL to about 80 mL, or about 4 mL to 60 mL, or from 5 mL to 40 mL, or from 6 mL to 20 mL (note these volumes apply to a SWIIN module comprising a, e.g., 50-500K perforation member). The volume of the reservoirs may range from 5 mL to 50 mL, or from 7 mL to 40 mL, or from 8 mL to 30 mL or from 10 mL to 20 mL, and the volumes of all reservoirs may be the same or the volumes of the reservoirs may differ (e.g., the volume of the permeate reservoirs is greater than that of the retentate reservoirs).

The serpentine channel portions 560a and 560b of the permeate member 508 and retentate member 504, respectively, are approximately 200 mm long, 130 mm wide, and 4 mm thick, though in other embodiments, the retentate and permeate members can be from 75 mm to 400 mm in length, or from 100 mm to 300 mm in length, or from 150 mm to 250 mm in length; from 50 mm to 250 mm in width, or from 75 mm to 200 mm in width, or from 100 mm to 150 mm in width; and from 2 mm to 15 mm in thickness, or from 4 mm to 10 mm in thickness, or from 5 mm to 8 mm in thickness. Embodiments the retentate (and permeate) members may be fabricated from PMMA (poly(methyl methacrylate) or other materials may be used, including polycarbonate, cyclic olefin co-polymer (COC), glass, polyvinyl chloride, polyethylene, polyamide, polypropylene, polysulfone, polyurethane, and co-polymers of these and other polymers. Preferably at least the retentate member is fabricated from a transparent material so that the cells can be visualized (see, e.g., FIG. 5I and the description thereof). For example, a video camera may be used to monitor cell growth by, e.g., density change measurements based on an image of an empty well, with phase contrast, or if, e.g., a chromogenic marker, such as a chromogenic protein, is used to add a distinguishable color to the cells. Chromogenic markers such as blitzen blue, dreidel teal, virginia violet, vixen purple, prancer purple, tinsel purple, maccabee purple, donner magenta, cupid pink, seraphina pink, scrooge orange, and leor orange (the Chromogenic Protein Paintbox, all available from ATUM (Newark, Calif.)) obviate the need to use fluorescence, although fluorescent cell markers, fluorescent proteins, and chemiluminescent cell markers may also be used.

Because the retentate member preferably is transparent, colony growth in the SWIIN module can be monitored by automated devices such as those sold by JoVE (ScanLag™ system, Cambridge, Mass.) (also see Levin-Reisman, et al., Nature Methods, 7:737-39 (2010)). Cell growth for, e.g., mammalian cells may be monitored by, e.g., the growth monitor sold by IncuCyte (Ann Arbor, Mich.) (see also, Choudhry, PLos One, 11(2):e0148469 (2016)). Further, automated colony pickers may be employed, such as those sold by, e.g., TECAN (Pickolo™ system, Mannedorf, Switzerland); Hudson Inc. (RapidPick™, Springfield, N.J.); Molecular Devices (QPix 400™ system, San Jose, Calif.); and Singer Instruments (PIXL™ system, Somerset, UK).

Due to the heating and cooling of the SWIIN module, condensation may accumulate on the retentate member which may interfere with accurate visualization of the growing cell colonies. Condensation of the SWIIN module 550 may be controlled by, e.g., moving heated air over the top of (e.g., retentate member) of the SWIIN module 550, or by applying a transparent heated lid over at least the serpentine channel portion 560b of the retentate member 504. See, e.g., FIG. 5I and the description thereof infra.

In SWIIN module 550 cells and medium—at a dilution appropriate for Poisson or substantial Poisson distribution of the cells in the microwells of the perforated member—are flowed into serpentine channel 560b from ports in retentate member 504, and the cells settle in the microwells while the medium passes through the filter into serpentine channel 560a in permeate member 508. The cells are retained in the microwells of perforated member 501 as the cells cannot travel through filter 503. Appropriate medium may be introduced into permeate member 508 through permeate ports 511. The medium flows upward through filter 503 to nourish the cells in the microwells (perforations) of perforated member 501. Additionally, buffer exchange can be effected by cycling medium through the retentate and permeate members. In operation, the cells are deposited into the microwells, are grown for an initial, e.g., 2-100 doublings, editing is induced by, e.g., raising the temperature of the SWIIN to 42° C. to induce a temperature inducible promoter or by removing growth medium from the permeate member and replacing the growth medium with a medium comprising a chemical component that induces an inducible promoter.

Once editing has taken place, the temperature of the SWIIN may be decreased, or the inducing medium may be removed and replaced with fresh medium lacking the chemical component thereby de-activating the inducible promoter. The cells then continue to grow in the SWIIN module 550 until the growth of the cell colonies in the microwells is normalized. For the normalization protocol, once the colonies are normalized, the colonies are flushed from the microwells by applying fluid or air pressure (or both) to the permeate member serpentine channel 560a and thus to filter 503 and pooled. Alternatively, if cherry picking is desired, the growth of the cell colonies in the microwells is monitored, and slow-growing colonies are directly selected; or, fast-growing colonies are eliminated.

FIG. 5D is a top perspective view of a SWIIN module with the retentate and perforated members in partial cross section. In this FIG. 5D, it can be seen that serpentine channel 560a is disposed on the top of permeate member 508 is defined by raised portions 576 and traverses permeate member 508 for most of the length and width of permeate member 508 except for the portion of permeate member 508 that comprises the permeate and retentate reservoirs (note only one retentate reservoir 552 can be seen). Moving from left to right, reservoir gasket 558 is disposed upon the integrated reservoir cover 578 (cover not seen in this FIG. 5D) of retentate member 504. Gasket 558 comprises reservoir access apertures 532a, 532b, 532c, and 532d, as well as pneumatic ports 533a, 533b, 533c and 533d. Also at the far left end is support 570. Disposed under permeate reservoir 552 can be seen one of two reservoir seals 562. In addition to the retentate member being in cross section, the perforated member 501 and filter 503 (filter 503 is not seen in this FIG. 5D) are in cross section. Note that there are a number of ultrasonic tabs 564 disposed at the right end of SWIIN module 550 and on raised portion 576 which defines the channel turns of serpentine channel 560a, including ultrasonic tabs 564 extending through through-holes 566 of perforated member 501. There is also a support 570 at the end distal reservoirs 552, 554 of permeate member 508.

FIG. 5E is a side perspective view of an assembled SWIIIN module 550, including, from right to left, reservoir gasket 558 disposed upon integrated reservoir cover 578 (not seen) of retentate member 504. Gasket 558 may be fabricated from rubber, silicone, nitrile rubber, polytetrafluoroethylene, a plastic polymer such as polychlorotrifluoroethylene, or other flexible, compressible material. Gasket 558 comprises reservoir access apertures 532a, 532b, 532c, and 532d, as well as pneumatic ports 533a, 533b, 533c and 533d. Also at the far-left end is support 570 of permeate member 508. In addition, permeate reservoir 552 can be seen, as well as one reservoir seal 562. At the far-right end is a second support 570.

FIG. 5F is a side perspective view of the reservoir portion of permeate member 508 and retentate member 504, including gasket 558. Seen are permeate reservoirs 552 as the outside reservoirs, and retentate reservoirs 554 between permeate reservoirs 552. It should be apparent to one of ordinary skill in the art given the present description, however, that this particular configuration of reservoirs may be changed with permeate 552 and retentate 554 reservoirs alternating in position; with both permeate reservoirs 552 on one side of SWIIN module 550 and both retentate reservoirs 554 on the other side of SWIIN module 550, or the retentate reservoirs 554 may be positioned at the two sides with the permeate reservoirs 552 between the retentate reservoirs. Again, gasket 558 comprises reservoir access apertures 532a, 532b, 532c, and 532d, as well as pneumatic ports 533a, 533b, 533c and 533d. In addition, two reservoir seals 562 can be seen, each sealing one permeate reservoir 552 and one retentate reservoir 554. Also seen is support 570 at the “reservoir end” of permeate member 508.

FIG. 5G is a side perspective cross sectional view of permeate reservoir 552 of permeate member 508 and retentate member 504 and gasket 558. Reservoir access aperture 532c and pneumatic aperture 533c can be seen, as well as support 570. Also seen is perforated member 501 and filter 503 (filter 503 is not seen clearly in this FIG. 5G but is sandwiched in between perforated member 501 and permeate member 508). A fluid path 572 from permeate reservoir 552 to serpentine channel 560a in permeate member 508 can be seen, as can reservoir seal 562.

FIG. 5H is a small segment of a cross section of SWIIN module 550, showing the retentate member 504, perforated member 501, filter 503, and retentate member 508. FIG. 5H also shows a fluid path 572 from a permeate reservoir to the serpentine channel 560a disposed in permeate member 508, and a fluid path 574 from a retentate reservoir to the serpentine channel 560b disposed in permeate member 504. As mentioned previously, the reservoir architecture of this embodiment is particularly advantageous as bubbling is minimized. That is, because the reservoirs and reservoir ports are positioned below the retentate and permeate serpentine channels, there is no instantaneous flow of fluid in the reservoirs into channels that connect the reservoir ports to the retentate and permeate channels. Instead, flow is controlled by the application of pressure (positive or negative) and an appropriate time chosen by the user.

Imaging of cell colonies growing in the wells of the SWIIN is desired in most implementations for, e.g., monitoring both cell growth and device performance and imaging is necessary for cherry-picking implementations. Real-time monitoring of cell growth in the SWIIN requires backlighting, retentate plate (top plate) condensation management and a system-level approach to temperature control, air flow, and thermal management. In some implementations, imaging employs a camera or CCD device with sufficient resolution to be able to image individual wells. For example, in some configurations a camera with a 9-pixel pitch is used (that is, there are 9 pixels center-to-center for each well). Processing the images may, in some implementations, utilize reading the images in grayscale, rating each pixel from low to high, where wells with no cells will be brightest (due to full or nearly-full light transmission from the backlight) and wells with cells will be dim (due to cells blocking light transmission from the backlight). After processing the images, thresholding is performed to determine which pixels will be called “bright” or “dim”, spot finding is performed to find bright pixels and arrange them into blocks, and then the spots are arranged on a hexagonal grid of pixels that correspond to the spots. Once arranged, the measure of intensity of each well is extracted, by, e.g., looking at one or more pixels in the middle of the spot, looking at several to many pixels at random or pre-set positions, or averaging X number of pixels in the spot. In addition, background intensity may be subtracted. Thresholding is again used to call each well positive (e.g., containing cells) or negative (e.g., no cells in the well). The imaging information may be used in several ways, including taking images at time points for monitoring cell growth. Monitoring cell growth can be used to, e.g., remove the “muffin tops” of fast-growing cells followed by removal of all cells or removal of cells in “rounds” as described above, or recover cells from specific wells (e.g., slow-growing cell colonies); alternatively, wells containing fast-growing cells can be identified and areas of UV light covering the fast-growing cell colonies can be projected (or rastered with shutters) onto the SWIIN to irradiate or inhibit growth of those cells. Imaging may also be used to assure proper fluid flow in the serpentine channel 560.

FIG. 5I depicts the embodiment of the SWIIN module in FIGS. 5A-5H further comprising a heat management system including a heater and a heated cover. The heater cover facilitates the condensation management that is required for imaging. Assembly 598 comprises a SWIIN module 550 seen lengthwise in cross section, where one permeate reservoir 552 is seen. Disposed immediately upon SWIIN module 550 is cover 594 and disposed immediately below SWIIN module 550 is backlight 580, which allows for imaging. Beneath and adjacent to the backlight and SWIIN module is insulation 582, which is disposed over a heatsink 584. In this FIG. 5I, the fins of the heatsink would be in-out of the page. In addition there is also axial fan 586 and heat sink 588, as well as two thermoelectric coolers 592, and a controller 590 to control the pneumatics, thermoelectric coolers, fan, solenoid valves, etc. The arrows denote cool air coming into the unit and hot air being removed from the unit. It should be noted that control of heating allows for growth of many different types of cells (prokaryotic and eukaryotic) as well as strains of cells that are, e.g., temperature sensitive, etc., and allows use of temperature-sensitive promoters. Temperature control allows for protocols to be adjusted to account for differences in transformation efficiency, cell growth and viability.

FIG. 5J is an exemplary pneumatic block diagram suitable for the SWIIN module depicted in FIGS. 5A-5I. In this configuration, there are two manifold arms that are controlled independently and there are two proportional valves instead of one, one each for the manifold arms. Tables 4-6 relate to the pneumatic diagram in FIG. 5J. Table 4 lists, for each step 1-32, the manifold arm status (open=arm open, closed=arm closed, motor engaged for pressurization); pump status (1: on, 0: off); energy status (1: energized, 0: de-energized) for each solenoid valve 1-4; and the pressure in psi for each proportional valve. Table 5 lists, for each step 1-32, the detection and threshold status for flow meters 1 and 2 as well as the duration of each step. When a change in pressure precedes a valve event, there is a delay of 1 second after reaching the set point before energizing the valves to avoid applying over- and under-shoots to the system. FALL=monitor for a falling signal, RISE=monitor for a rising signal. “Requires pLLD”=requires pressure-driven liquid level detection, such as, e.g., via air-displacement pipettor. Table 6 lists, for each step 1-32, the volumes for each reservoir, permeate reservoirs 1 and 2, and retentate reservoirs 1 and 2; the temperature of the SWIIN; and notes for operation. For details regarding SWIIN modules and methods see, e.g., U.S. Ser. No. 62/718,449, filed 14 Aug. 2018; 62/735,365, filed 24 Sep. 2018; 62/781,112, filed 13 Dec. 2018; 62/779,119, filed 13 Dec. 2018; 62/841,213, filed 30 Apr. 2019; Ser. No. 16/399,988, filed 30 Apr. 2019; Ser. No. 16/454,865, filed 26 Jun. 2019; and Ser. No. 16/540,606, filed 14 Aug. 2019.

### Use of the TFF Module in Exemplary Automated Multi-Module Cell Processing Instruments

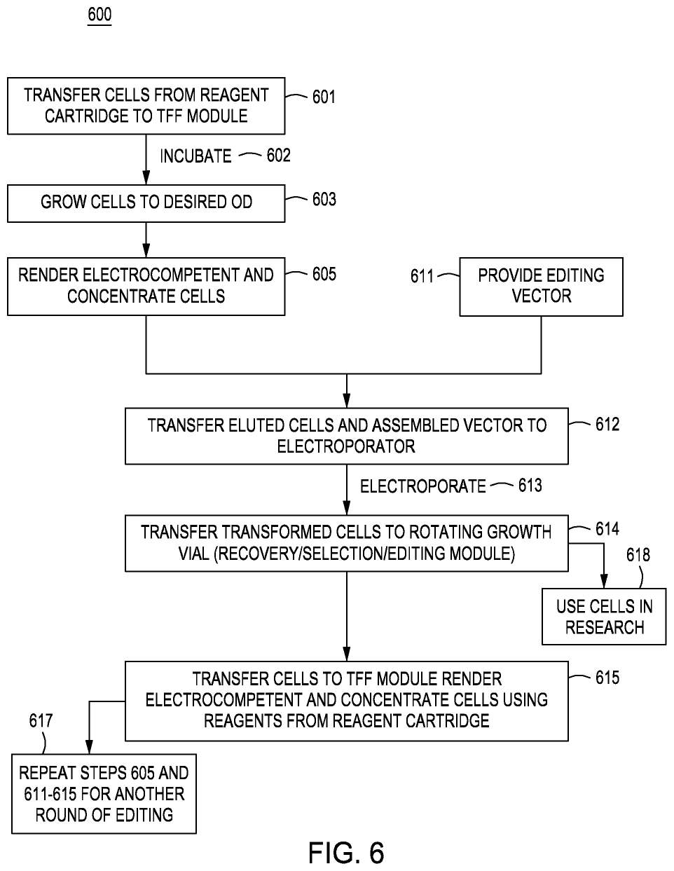

FIG. 6 is a block diagram of one embodiment of a method 600 for using the automated multi-module cell processing instrument 200 of FIG. 2, including the TFF modules described in relation to FIGS. 1B-1EE. In a first step, cells are transferred 601 from reagent cartridge 210 to TFF module 222. (Please see FIG. 2 in relation to element numbers in the two hundreds.) The cells are incubated or grown 602, e.g., until they grow to a desired OD 603. The cells are then concentrated and medium or buffer exchange is performed to, e.g., render the cells competent (e.g., electrocompetent) while also reducing the volume of the cell sample to a volume appropriate for electroporation, as well as to remove unwanted components, e.g., salts, from the cell sample. Once the cells have been rendered competent and suspended in an appropriate volume for transformation, the cell sample is transferred 612 to flow-through electroporation device 230 in reagent cartridge 210.

While cells are being processed for electroporation, pre-assembled vector backbones+expression/editing cassettes (e.g., editing vectors, including libraries or editing vectors) are provided 611 and are transferred to the flow-through electroporation device.

After electroporation 613, the transformed cells optionally are transferred 614 to a growth vial 220 to, e.g., recover from the transformation process and be submitted to selection and editing. Once the transformed cells have recovered, been selected (e.g., by an antibiotic or other reagent added from the reagent cartridge) and/or genome editing has taken place, the transformed cells may be removed from the instrument and used in further research 618, or transferred 615 into the TFF module 222 for buffer or medium exchange and/or to be concentrated and rendered competent for another round of transformation. The competent cells may then be collected in an empty vessel 206 in the wash cartridge. All or some of steps 601-605 and 611-615 may be repeated for recursive rounds of genome editing 617.

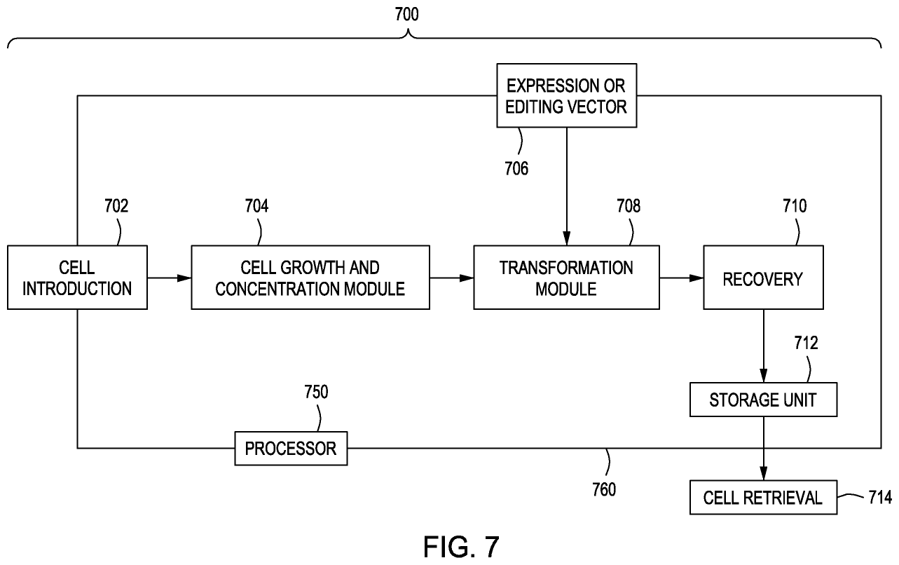

As described above, the reagent cartridges are used as components in an automated multi-module processing instrument. A general exemplary embodiment of a multi-module cell processing diagram is shown in FIG. 7. In some embodiments, the cell processing instrument 700 may include a housing 760, a receptacle for introducing cells to be transformed or transfected 702, and a TFF module 704. The cells to be transformed are transferred from a reagent cartridge or tube to the TFF module to be grown until the cells hit a target OD. Once the cells hit the target OD, the TFF module optionally may cool the cells for later processing and then concentrate (i.e., the volume of the cells is reduced to a volume appropriate for transformation) and render the cells competent (perform buffer exchange). The TFF module 704 performs cell growth to a desired OD, medium exchange to make the cells competent, and reduction of the volume of the competent cells. In one example of buffer exchange and cell concentration, 20 ml of cells+growth media is concentrated to 400 μl cells in 10% glycerol. Once the competent cells have been concentrated, the cells are transferred to, e.g., an electroporation device (a transformation module 708) to be transformed with a desired nucleic acid(s). In addition to the receptacle for receiving cells, the multi-module cell processing instrument may include a receptacle located in the reagent cartridge for storing the nucleic acids to be electroporated into the cells 706. The nucleic acids are transferred to, e.g., the transformation module 708—such as a flow-through electroporation device—which already contains the concentrated competent cells grown to the specified OD, and the nucleic acids are introduced into the cells. Following electroporation, the transformed cells are transferred into, e.g., a recovery module 710. Here, the cells are allowed to recover from the electroporation procedure.

In some embodiments, after recovery the cells are transferred to a storage module 712 to be stored at, e.g., 4° C. or frozen. The cells can then be retrieved from a retrieval module 714 and, e.g., used for protein expression or other studies performed off-line. The automated multi-module cell processing instrument is controlled by a processor 750 configured to operate the instrument based on user input and/or one or more scripts, which may be associated with the reagent cartridge or other module. The processor 750 may control the timing, duration, temperature, and other operations (including, e.g., dispensing reagents) of the various modules of the instrument 700 as specified by one or more scripts. In addition to or as an alternative to the one or more scripts, the processor may be programmed with standard protocol parameters from which a user may select; alternatively, a user may select one or all parameters manually. The script may specify, e.g., the wavelength at which OD is read in the TFF module, the target OD to which the cells are grown, the target time at which the cells will reach the target OD, and/or the volume to which the cells should be concentrated. The processor may update the user (e.g., via an application to a smart phone or other device) as to the progress of the cells in the cell growth module, electroporation device, filtration module, recovery module, etc. in the automated multi-module cell processing instrument.

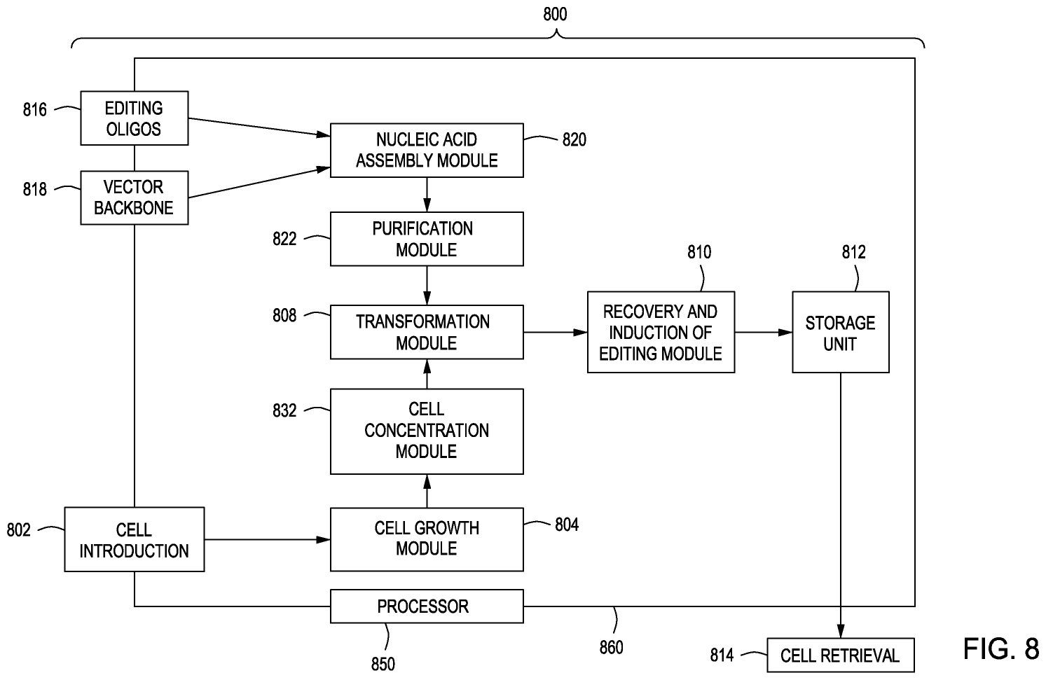

A second embodiment of an automated multi-module cell processing instrument is shown in FIG. 8, where this embodiment is drawn to nucleic acid-guided nuclease editing. As with the embodiment shown in FIG. 7, the cell processing instrument 800 may include a housing 860, a reservoir of cells in, e.g., the reagent cartridge to be transformed or transfected 802, and a cell growth module 804, separate from the cell concentration module (TFF) 824. The cells to be processed are transferred from, e.g., a reservoir in the reagent cartridge to the cell growth module 804 to be cultured until the cells hit a target OD. In this embodiment, the cells are grown in a, e.g., rotating growth vial in a cell growth module separate from the TFF. Once the cells hit the target OD, the cell growth module may cool or freeze the cells for later processing. After growth, the cells may be transferred to the TFF 832, in this instance, a separate module from the cell growth module 804, where buffer or medium exchange is performed, the cells are rendered competent, and the volume of the cells is reduced to a volume optimal for cell transformation in a TFF. Once concentrated, the cells are then transferred to the transformation module in the reagent cartridge 808 (e.g., an electroporation device).

In addition to the reservoir for storing the cells, the reagent cartridge may include a reservoir for storing editing cassettes 816 and a reservoir for storing a vector backbone 818. Both the editing cassettes and the vector backbone are transferred from the reagent cartridge to a nucleic acid assembly module 820, where the editing cassettes are inserted into the vector backbone. The assembled nucleic acids may be transferred into an optional purification module 822 for desalting and/or other purification procedures needed to prepare the assembled nucleic acids for transformation. Once the processes carried out by the assembly/purification module 822 are complete, the assembled nucleic acids are transferred to a transformation module 808, which already contains the cell culture grown to a target OD, rendered competent and concentrated. In the transformation module 808, the nucleic acids are introduced into the cells. Following transformation, the cells are transferred into a combined recovery and editing module 812. As described above, in some embodiments the automated multi-module cell processing instrument 800 is a system that performs gene editing such as an RNA-direct nuclease editing system. For examples of multi-module cell editing instruments, see U.S. Pat. No. 10,253,316, issued 9 Apr. 2019; U.S. Pat. No. 10,329,559, issued 25 Jun. 2019; and U.S. Pat. No. 10,323,242, issued 18 Jun. 2019; and U.S. Ser. No. 16/412,175, filed 14 May 2019; Ser. No. 16/412,195, filed 14 May 2019; and Ser. No. 16/423,289, filed 28 May 2019, all of which are herein incorporated by reference in their entirety. In the recovery and editing module 810, the cells are allowed to recover post-transformation, and the cells express the nuclease and editing oligonucleotides to effect editing in desired genes in the cells.

Following editing, the cells are transferred to a storage module 814, where the cells can be stored at, e.g., 4° C. until the cells are retrieved for further study. The multi-module cell processing instrument is controlled by a processor 850 configured to operate the instrument based on user input, as directed by one or more scripts, or as a combination of user input or a script. The processor 850 may control the timing, duration, temperature, and operations of the various modules of the instrument 800 and the dispensing of reagents from the reagent cartridge. The processor may be programmed with standard protocol parameters from which a user may select, a user may specify one or more parameters manually or one or more scripts associated with the reagent cartridge may specify one or more operations and/or reaction parameters. In addition, the processor may notify the user (e.g., via an application to a smart phone or other device) that the cells have reached a target OD, been rendered competent and concentrated, and/or update the user as to the progress of the cells in the various modules in the multi-module instrument.

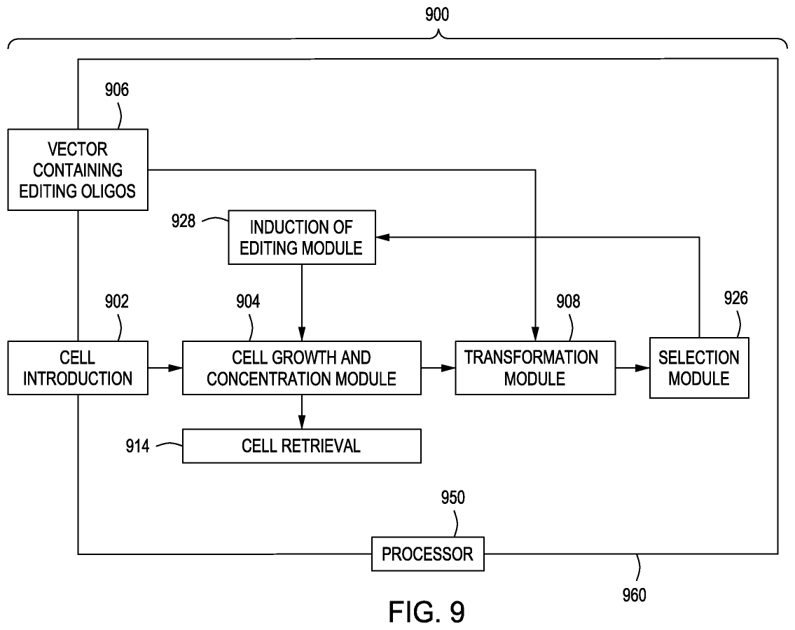

As described above, in one embodiment the automated multi-module cell processing instrument 800 is a nucleic acid-guided nuclease editing system. Multiple nuclease-based systems exist for providing edits into a cell and each can be used in either single editing systems as could be performed in the automated instrument 700 of FIG. 7 or 800 of FIG. 8; and/or sequential editing systems as could be performed in the automated instrument 900 of FIG. 9 described below, e.g., using different nucleic acid-guided nuclease systems sequentially to provide two or more genome edits in a cell; and/or recursive editing systems as could be performed in the automated instrument 900 of FIG. 9, e.g. utilizing a single nuclease-directed system to introduce two or more genome edits in a cell. Automated nuclease-directed processing instruments use the nucleases to cleave the cell's genome, to introduce one or more edits into a target region of the cell's genome, or both. Nuclease-directed genome editing mechanisms may include zinc-finger editing mechanisms (see Urnov et al., Nature Reviews Genetics, 11:636-64 (2010)), meganuclease editing mechanisms (see Epinat et al., Nucleic Acids Research, 31(11):2952-62 (2003); and Arnould et al., Journal of Molecular Biology, 371(1):49-65 (2007)), and RNA-guided editing mechanisms (see Jinek et al., Science, 337:816-21 (2012); and Mali et al, Science, 339:823-26 (2013)). In particular embodiments, the nucleic acid-guided nuclease system is an inducible system that allows control of the timing of the editing (see U.S. Ser. No. 16/454,865, filed 26 Jun. 2019). That is, when the cell or population of cells comprising a nucleic acid-guided nuclease encoding DNA is in the presence of the inducer molecule, expression of the nuclease can occur. The ability to modulate nuclease activity can reduce off-target cleavage and facilitate precise genome engineering.

A third embodiment of a multi-module cell processing instrument is shown in FIG. 9. This embodiment depicts an exemplary system that performs recursive gene editing on a cell population. As with the embodiment shown in FIGS. 7 and 8, the cell processing system 900 may include a housing 960, a reservoir in a reagent cartridge for storing cells to be transformed or transfected 902, and a TFF module 904. The cells to be transformed are transferred from a reservoir in the reagent cartridge to the TFF module 904 to be cultured until the cells hit a target OD. Once the cells hit the target OD, the TFF module (cell growth and concentration module) 904 renders the cells competent and reduces the volume of the cells. Once the cells have been concentrated to an appropriate volume, the cells are transferred to a transformation module 908. In addition, the assembled nucleic acids are transferred to the transformation module 908, which already contains the cell culture grown to a target OD. In the transformation module 908, the nucleic acids are introduced into the cells. Following transformation, the cells are transferred into a selection module 926.

After selection, the cells may be transferred to an editing module 928 where providing conditions for the cells to edit, e.g., if editing is driven by an inducible promoter. After editing, the cells are transferred back to a TFF module 904 where the edited cells are allowed to grow, and then buffer or medium exchange is performed once again and the cells are rendered competent once again in preparation for transfer to the transformation module 908. Note that in the case of a SWIIN, for example, selection, editing and growth all take place in the same module.

In transformation module 908, the cells are transformed by a second set of editing cassettes (or other type of cassette) and the cycle is repeated until the cells have been transformed and edited by a desired number of, e.g., editing cassettes. As discussed above in relation to FIGS. 7 and 8, the exemplary multi-module cell processing instrument is controlled by a processor 950 configured to operate the instrument based on user input, or is controlled by one or more scripts, for example, one or more scripts associated with the reagent cartridge. The processor 950 may control the timing, duration, and temperature of various processes, the dispensing of reagents, and other operations of the various modules of the instrument 900. For example, a script or the processor may control the dispensing of cells, reagents, vectors, and editing cassettes; which editing cassettes are used for cell editing and in what order; the time, temperature and other conditions used in the recovery and expression module, the wavelength at which OD is read in the TFF module, the target OD to which the cells are grown, the target time at which the cells will reach the target OD, and/or the volume to which the cells are concentrated. In addition, the processor may be programmed to notify a user (e.g., via an application) as to the progress of the cells in the automated multi-module cell processing instrument.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Other equivalent methods, steps and compositions are intended to be included in the scope of the invention. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric.

### Example 1: Cell Culture

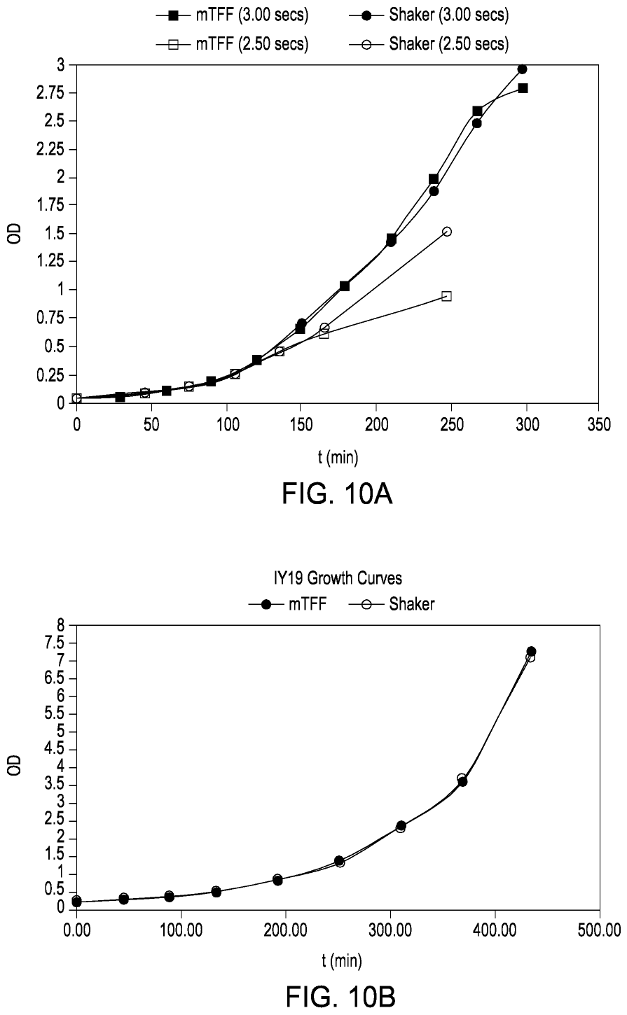

Both bacterial (E. coli) and yeast (S. cerevisae) cells were cultures in the TFF device. E. coli cells were grown on LB medium with 25 μg/mL chloramphenicol, and S. cerevisae were grown in YDP medium with 100 μg/mL carbenicol. For both E. coli and S. cerevisae, starter culture was grown overnight, and a 1/100 dilution of the starter cultures were grown in 30 mL of the appropriate medium in the device. The initial culture was loaded into one of the retentate reservoirs. Bubbling of the culture at 20 psi was performed while the cell cultures resided in the reservoirs. The cultures were then transferred through the flow channel at 30 psi to the other retentate reservoir where bubbling (aeration) of the cultures at 20 psi effected mixing. The transfer and bubbling processes were repeated several times. Fresh medium was added to the cell cultures as old medium was removed from the cultures during the transfer of the cell culture through the flow channel. The results of culturing E. coli in the TFF device vs. a traditional shaker culture is shown in FIG. 10A. Note that although the shaker protocol produced faster growth with only 2.5 seconds bubbling in the device, the TFF and shaker protocols were virtually identical with 300 second of bubbling in the device. FIG. 10B shows the results of cell growth (measured by optical density) of S. cerevisae using both TFF and shake protocols, where cell growth in these methods was virtually identical.

### Example 2: Cell Concentration

The TFF module as described above in relation to FIGS. 1B-1EE has been used successfully to process and perform buffer exchange on both E. coli and yeast cultures. In concentrating an E. coli culture, the following steps were performed:

First, a 20 ml culture of E. coli in LB grown to OD 0.5-0.62 was passed through the TFF device in one direction, then passed through the TFF device in the opposite direction. At this point the cells were concentrated to a volume of approximately 5 ml. Next, 50 ml of 10% glycerol was added to the concentrated cells, and the cells were passed through the TFF device in one direction, in the opposite direction, and back in the first direction for a total of three passes. Again the cells were concentrated to a volume of approximately 5 ml. Again, 50 ml of 10% glycerol was added to the 5 ml of cells and the cells were passed through the TFF device for three passes. This process was repeated; that is, again 50 ml 10% glycerol was added to cells concentrated to 5 ml, and the cells were passed three times through the TFF device. At the end of the third pass of the three 50 ml 10% glycerol washes, the cells were again concentrated to approximately 5 ml of 10% glycerol. The cells were then passed in alternating directions through the TFF device three more times, wherein the cells were concentrated into a volume of approximately 400 μl.

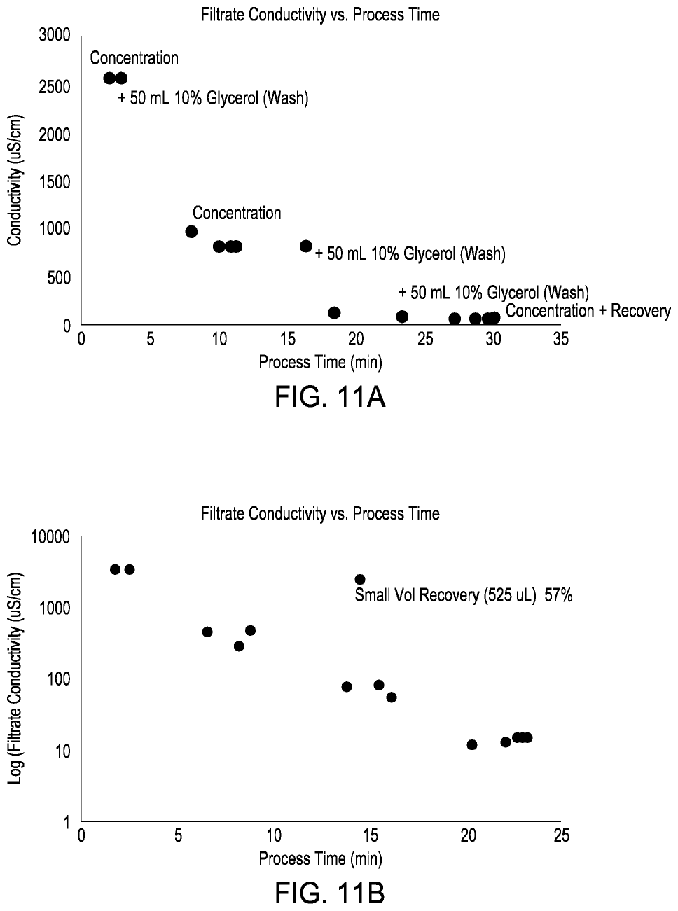

FIG. 11A presents a graph showing filter buffer exchange performance for E. coli determined by measuring filtrate conductivity and filter processing time. Filter performance is quantified by measuring the time and number of filter passes required to obtain a target solution electrical conductivity. Cell retention is determined by comparing the optical density (OD600) of the cell culture both before and after filtration. Filter health is monitored by measuring the transmembrane flow rate during each filter pass. As seen in FIG. 11A, target conductivity (˜16 μS/cm) was achieved in approximately 30 minutes utilizing three 50 ml 10% glycerol washes and three passes of the cells through the device for each wash. The volume of the cells was reduced from 20 ml to 400 μl, and recovery of approximately 90% of the cells has been achieved.

The same process was repeated with yeast cell cultures. A yeast culture was initially concentrated to approximately 5 ml using two passes through the TFF device in opposite directions. The cells were washed with 50 ml of 1M sorbitol three times, with three passes through the TFF device after each wash. After the third pass of the cells following the last wash with 1M sorbitol, the cells were passed through the TFF device two times, wherein the yeast cell culture was concentrated to approximately 525 μl. FIG. 11B presents the filter buffer exchange performance for yeast cells determined by measuring filtrate conductivity and filter processing time. Target conductivity (˜10 μS/cm) was achieved in approximately 23 minutes utilizing three 50 ml 1M sorbitol washes and three passes through the TFF device for each wash. The volume of the cells was reduced from 20 ml to 525 μl. Recovery of approximately 90% of the cells has been achieved.

### Example 3: Fully-Automated Singleplex RGN-Directed Editing Run

Singleplex automated genomic editing using MAD7 nuclease was successfully performed with an automated multi-module instrument such as that shown in FIGS. 2A-2D. See U.S. Pat. No. 9,982,279, issued 29 May 2018 and Ser. No. 10/240,167, issued 9 Apr. 2019.

An ampR plasmid backbone and a lacZ_F172* editing cassette were assembled via Gibson Assembly® into an “editing vector” in an isothermal nucleic acid assembly module included in the automated instrument. lacZ_F172 functionally knocks out the lacZ gene. “lacZ_F172*” indicates that the edit happens at the 172nd residue in the lacZ amino acid sequence. Following assembly, the product was de-salted in the isothermal nucleic acid assembly module using AMPure beads, washed with 80% ethanol, and eluted in buffer. The assembled editing vector and recombineering-ready, electrocompetent E. Coli cells were transferred into a transformation module for electroporation. The cells and nucleic acids were combined and allowed to mix for 1 minute, and electroporation was performed for 30 seconds. The parameters for the poring pulse were: voltage, 2400 V; length, 5 ms; interval, 50 ms; number of pulses, 1; polarity, +. The parameters for the transfer pulses were: Voltage, 150 V; length, 50 ms; interval, 50 ms; number of pulses, 20; polarity, +/−. Following electroporation, the cells were transferred to a recovery module (another growth module) and allowed to recover in SOC medium containing chloramphenicol. Carbenicillin was added to the medium after 1 hour, and the cells were allowed to recover for another 2 hours. After recovery, the cells were held at 4° C. until recovered by the user.

After the automated process and recovery, an aliquot of cells was plated on MacConkey agar base supplemented with lactose (as the sugar substrate), chloramphenicol and carbenicillin and grown until colonies appeared. White colonies represented functionally edited cells, purple colonies represented un-edited cells. All liquid transfers were performed by the automated liquid handling device of the automated multi-module cell processing instrument.

The result of the automated processing was that approximately 1.0E⁻⁰³ total cells were transformed (comparable to conventional benchtop results), and the editing efficiency was 83.5%. The lacZ_172 edit in the white colonies was confirmed by sequencing of the edited region of the genome of the cells. Further, steps of the automated cell processing were observed remotely by webcam and text messages were sent to update the status of the automated processing procedure.

### Example 4: Fully-Automated Recursive Editing Run

Recursive editing was successfully achieved using the automated multi-module cell processing system. An amp^{R} plasmid backbone and a lacZ_V10* editing cassette were assembled via Gibson Assembly® into an “editing vector” in an isothermal nucleic acid assembly module included in the automated system. Similar to the lacZ_F172 edit, the lacZ_V10 edit functionally knocks out the lacZ gene. “lacZ_V10” indicates that the edit happens at amino acid position 10 in the lacZ amino acid sequence. Following assembly, the product was de-salted in the isothermal nucleic acid assembly module using AMPure beads, washed with 80% ethanol, and eluted in buffer. The first assembled editing vector and the recombineering-ready electrocompetent E. Coli cells were transferred into a transformation module for electroporation. The cells and nucleic acids were combined and allowed to mix for 1 minute, and electroporation was performed for 30 seconds. The parameters for the poring pulse were: voltage, 2400 V; length, 5 ms; interval, 50 ms; number of pulses, 1; polarity, +. The parameters for the transfer pulses were: Voltage, 150 V; length, 50 ms; interval, 50 ms; number of pulses, 20; polarity, +/−. Following electroporation, the cells were transferred to a recovery module (another growth module) allowed to recover in SOC medium containing chloramphenicol. Carbenicillin was added to the medium after 1 hour, and the cells were grown for another 2 hours. The cells were then transferred to a centrifuge module and a media exchange was then performed. Cells were resuspended in TB containing chloramphenicol and carbenicillin where the cells were grown to OD600 of 2.7, then concentrated and rendered electrocompetent.

During cell growth, a second editing vector was prepared in the isothermal nucleic acid assembly module. The second editing vector comprised a kanamycin resistance gene, and the editing cassette comprised a galK Y145* edit. If successful, the galK Y145* edit confers on the cells the ability to uptake and metabolize galactose. The edit generated by the galK Y154* cassette introduces a stop codon at the 154th amino acid residue, changing the tyrosine amino acid to a stop codon. This edit makes the galK gene product non-functional and inhibits the cells from being able to metabolize galactose. Following assembly, the second editing vector product was de-salted in the isothermal nucleic acid assembly module using AMPure beads, washed with 80% ethanol, and eluted in buffer. The assembled second editing vector and the electrocompetent E. Coli cells (that were transformed with and selected for the first editing vector) were transferred into a transformation module for electroporation, using the same parameters as detailed above. Following electroporation, the cells were transferred to a recovery module (another growth module), allowed to recover in SOC medium containing carbenicillin. After recovery, the cells were held at 4° C. until retrieved, after which an aliquot of cells were plated on LB agar supplemented with chloramphenicol, and kanamycin. To quantify both lacZ and galK edits, replica patch plates were generated on two media types: 1) MacConkey agar base supplemented with lactose (as the sugar substrate), chloramphenicol, and kanamycin, and 2) MacConkey agar base supplemented with galactose (as the sugar substrate), chloramphenicol, and kanamycin. All liquid transfers were performed by the automated liquid handling device of the automated multi-module cell processing system.

In this recursive editing experiment, 41% of the colonies screened had both the lacZ and galK edits, the results of which were comparable to the double editing efficiencies obtained using a “benchtop” or manual approach.

While this invention is satisfied by embodiments in many different forms, as described in detail in connection with preferred embodiments of the invention, it is understood that the present disclosure is to be considered as exemplary of the principles of the invention and is not intended to limit the invention to the specific embodiments illustrated and described herein. Numerous variations may be made by persons skilled in the art without departure from the spirit of the invention. The scope of the invention will be measured by the appended claims and their equivalents. The abstract and the title are not to be construed as limiting the scope of the present invention, as their purpose is to enable the appropriate authorities, as well as the general public, to quickly determine the general nature of the invention. In the claims that follow, unless the term “means” is used, none of the features or elements recited therein should be construed as means-plus-function limitations pursuant to 35 U.S.C. § 112, 916.

TABLE 1
Valve Status and Pressure

| | | | | Valve 1 | Valve 2 | Valve 3 | Valve 4 | Valve 5 | Press 1 | Press 2 | Detect |
|---|---|---|---|---|---|---|---|---|---|---|---|
| Description of step | Step | Manifold | pump | (RR1) | (RR2) | (EBR) | (pump) | (pump) | (psi) | (psi) | Spike? |
| Load TFF cartridge | 1 | open | 0 | 0 | 0 | 0 | 0 | 0 | 0 | 0 | NA |
| on instrument | | | | | | | | | | | |
| Transfer 20 mL cell | 2 | open | 0 | 0 | 0 | 0 | 0 | 0 | 0 | 0 | NA |
| culture to RR1 | | | | | | | | | | | |
| Close manifold | 3 | closed | 0 | 0 | 0 | 0 | 0 | 0 | 0 | 0 | NA |
| Concentration pass | 4 | closed | 1 | 1 | 0 | 0 | 0 | 0 | 24 | 24 | YES |
| Concentration pass | 5 | closed | 1 | 0 | 1 | 0 | 0 | 0 | 24 | 24 | YES |
| Concentration pass | 6 | closed | 1 | 1 | 0 | 0 | 0 | 0 | 24 | 24 | YES |
| Concentration pass | 7 | closed | 1 | 0 | 1 | 0 | 0 | 0 | 20 | 20 | YES |
| Concentration pass | 8 | closed | 1 | 1 | 0 | 0 | 0 | 0 | 10 | 10 | YES |
| Open manifold and | 9 | open | 0 | 0 | 0 | 0 | 0 | | 0 | 0 | NA |
| transfer treatment | | | | | | | | | | | |
| buffer to RR2 | | | | | | | | | | | |
| Close manifold | 10 | closed | 0 | 0 | 0 | 0 | 0 | 0 | 0 | 0 | NA |
| Transfer to RR1 | 11 | closed | 1 | 1 | 0 | 1 | 1 | 1 | −10 | 0 | YES |
| Bubble 30 min | 12 | closed | 1 | 0 | 1 | 0 | 0 | 0 | 20 | 0 | NO |
| Concentration pass | 13 | closed | 1 | 1 | 0 | 0 | 0 | 0 | 24 | 24 | YES |
| Concentration pass | 14 | closed | 1 | 0 | 1 | 0 | 0 | 0 | 24 | 24 | YES |
| Concentration pass | 15 | closed | 1 | 1 | 0 | 0 | 0 | 0 | 24 | 24 | YES |
| Concentration pass | 16 | closed | 1 | 0 | 1 | 0 | 0 | 0 | 20 | 20 | YES |
| Concentration pass | 17 | closed | 1 | 1 | 0 | 0 | 0 | 0 | 10 | 10 | YES |
| Transfer wash fluid | 18 | closed | 1 | 0 | 0 | 1 | 0 | 0 | 10 | 0 | NO |
| from EBR to RR2 | | | | | | | | | | | |
| Transfer to RR1and | 19 | closed | 1 | 1 | 0 | 1 | 1 | 1 | −10 | 0 | YES |
| bubble 2.5 secs | | | | | | | | | | | |
| Concentration pass | 20 | closed | 1 | 1 | 0 | 0 | 0 | 0 | 24 | 24 | YES |
| Concentration pass | 21 | closed | 1 | 0 | 1 | 0 | 0 | 0 | 24 | 24 | YES |
| Concentration pass | 22 | closed | 1 | 1 | 0 | 0 | 0 | 0 | 24 | 24 | YES |
| Concentration pass | 23 | closed | 1 | 0 | 1 | 0 | 0 | 0 | 20 | 20 | YES |
| Concentration pass | 24 | closed | 1 | 1 | 0 | 0 | 0 | 0 | 10 | 10 | YES |
| Transfer wash fluid | 25 | closed | 1 | 0 | 0 | 1 | 0 | 0 | 10 | 0 | NO |
| from EBR to RR2 | | | | | | | | | | | |
| Transfer to RR1and | 26 | closed | 1 | 1 | 0 | 1 | 1 | 1 | −10 | 0 | YES |
| bubble 2.5 secs 0 | | | | | | | | | | | |
| Concentration pass | 27 | closed | 1 | 1 | 0 | 0 | 0 | 0 | 24 | 24 | YES |
| Concentration pass | 28 | closed | 1 | 0 | 1 | 0 | 0 | 0 | 24 | 24 | YES |
| Concentration pass | 29 | closed | 1 | 1 | 0 | 0 | 0 | 0 | 24 | 24 | YES |
| Concentration pass | 30 | closed | 1 | 0 | 1 | 0 | 0 | 0 | 20 | 20 | YES |
| Concentration pass | 31 | closed | 1 | 1 | 0 | 0 | 0 | 0 | 10 | 10 | YES |
| Transfer wash fluid | 32 | closed | 1 | 0 | 0 | 1 | 0 | | 10 | 0 | NO |
| from EBR to RR2 | | | | | | | | | | | |
| Transfer to RR1and | 33 | closed | 1 | 1 | 0 | 1 | 1 | 1 | −10 | 0 | YES |
| bubble 2.5 secs | | | | | | | | | | | |
| Concentration pass | 34 | closed | 1 | 1 | 0 | 0 | 0 | 0 | 24 | 24 | YES |
| Concentration pass | 35 | closed | 1 | 0 | 1 | 0 | 0 | 0 | 24 | 24 | YES |
| Concentration pass | 36 | closed | 1 | 1 | 0 | 0 | 0 | 0 | 24 | 24 | YES |
| Concentration pass | 37 | closed | 1 | 0 | 1 | 0 | 0 | 0 | 20 | 20 | YES |
| Concentration pass | 38 | closed | 1 | 1 | 0 | 0 | 0 | 0 | 10 | 10 | YES |
| Elution from | 39 | closed | 1 | 1 | 1 | 1 | 1 | 1 | −10 | −10 | NO |
| permeate side | | | | | | | | | | | |
| Sweep to RR1 | 40 | closed | 1 | 1 | 0 | 1 | 1 | 1 | −5 | 0 | NO |
| Open manifold and | 41 | open | 0 | 0 | 0 | 0 | 0 | 0 | 0 | 0 | NA |
| recover culture | | | | | | | | | | | |
| from RR1 | | | | | | | | | | | |

TABLE 2
Reservoir Volumes

| Description of step | Step | RR1 I | RR1 F | RR2 I | RR2 F | EBR I | EBR F | PR I | PR F | Temp (° C.) |
|---|---|---|---|---|---|---|---|---|---|---|
| Load TFF cartridge | 1 | 0 | 0 | 0 | 0 | 75 | 75 | 0.0 | 0.0 | TBD |
| on instrument | | | | | | | | | | |
| Transfer 20 mL cell | 2 | 0 | 20 | 0 | 0 | 75 | 75 | 0.0 | 0.0 | 30 |
| culture to RR1 | | | | | | | | | | |
| Close manifold | 3 | 20 | 20 | 0 | 0 | 75 | 75 | 0.0 | 0.0 | 30 |
| Concentration pass | 4 | 20 | 0 | 0 | 7 | 75 | 75 | 0.0 | 13.0 | 30 |
| Concentration pass | 5 | 0 | 3 | 7 | 0 | 75 | 75 | 13.0 | 17.0 | 30 |
| Concentration pass | 6 | 3 | 0 | 0 | 1 | 75 | 75 | 17.0 | 19.0 | 30 |
| Concentration pass | 7 | 0 | 0.4 | 1 | 0 | 75 | 75 | 19.0 | 19.6 | 30 |
| Concentration pass | 8 | 0.4 | 0 | 0 | 0.3 | 75 | 75 | 19.6 | 19.7 | 30 |
| Open manifold and | 9 | 0 | 0 | 0.3 | 10.3 | 75 | 75 | 19.7 | 19.7 | 30 |
| transfer treatment | | | | | | | | | | |
| buffer to RR2 | | | | | | | | | | |
| Close manifold | 10 | 0 | 0 | 10.3 | 10.3 | 75 | 75 | 19.7 | 19.7 | 4 |
| Transfer to RR1 | 11 | 0 | 10.3 | 10.3 | 0 | 75 | 75 | 19.7 | 19.7 | 4 |
| Bubble 30 min | 12 | 10.3 | 10.3 | 0 | 0 | 75 | 75 | 19.7 | 19.7 | 4 |
| Concentration pass | 13 | 10.3 | 0 | 0 | 4 | 75 | 75 | 19.7 | 26.0 | 4 |
| Concentration pass | 14 | 0 | 1.5 | 4 | 0 | 75 | 75 | 26.0 | 28.5 | 4 |
| Concentration pass | 15 | 1.5 | 0 | 0 | 0.6 | 75 | 75 | 28.5 | 29.4 | 4 |
| Concentration pass | 16 | 0 | 0.4 | 0.6 | 0 | 75 | 75 | 29.4 | 29.6 | 4 |
| Concentration pass | 17 | 0.4 | 0 | 0 | 0.3 | 75 | 75 | 29.6 | 29.7 | 4 |
| Transfer wash fluid | 18 | 0 | 0 | 0.3 | 20.3 | 75 | 55 | 29.7 | 29.7 | 4 |
| from EBR to RR2 | | | | | | | | | | |
| Transfer to RR1and | 19 | 0 | 0.3 | 20.3 | 0 | 55 | 55 | 29.7 | 29.7 | 4 |
| bubble 2.5 secs | | | | | | | | | | |
| Concentration pass | 20 | 20.3 | 0 | 0 | 7 | 55 | 55 | 29.7 | 43.0 | 4 |
| Concentration pass | 21 | 0 | 3 | 7 | 0 | 55 | 55 | 43.0 | 47.0 | 4 |
| Concentration pass | 22 | 3 | 0 | 0 | 1 | 55 | 55 | 47.0 | 49.0 | 4 |
| Concentration pass | 23 | 0 | 0.4 | 1 | 0 | 55 | 55 | 49.0 | 49.6 | 4 |
| Concentration pass | 24 | 0.4 | 0 | 0 | 0.3 | 55 | 55 | 49.6 | 49.7 | 4 |
| Transfer wash fluid | 25 | 0 | 0 | 0.3 | 20.3 | 55 | 35 | 49.7 | 49.7 | 4 |
| from EBR to RR2 | | | | | | | | | | |
| Transfer to RR1and | 26 | 0 | 20.3 | 20.3 | 0 | 35 | 35 | 49.7 | 49.7 | 4 |
| bubble 2.5 secs 0 | | | | | | | | | | |
| Concentration pass | 27 | 20.3 | 0 | 0 | 7 | 35 | 35 | 49.7 | 63.0 | 4 |
| Concentration pass | 28 | 0 | 3 | 7 | 0 | 35 | 35 | 63.0 | 67.0 | 4 |
| Concentration pass | 29 | 3 | 0 | 0 | 1 | 35 | 35 | 67.0 | 69.0 | 4 |
| Concentration pass | 30 | 0 | 0.4 | 1 | 0 | 35 | 35 | 69.0 | 69.6 | 4 |
| Concentration pass | 31 | 0.4 | 0 | 0 | 0.3 | 35 | 35 | 69.6 | 69.7 | 4 |
| Transfer wash fluid | 32 | 0 | 0 | 0.3 | 20.3 | 35 | 15 | 69.7 | 69.7 | 4 |
| from EBR to RR2 | | | | | | | | | | |
| Transfer to RR1and | 33 | 0 | 20.3 | 20.3 | 0 | 15 | 15 | 69.7 | 69.7 | 4 |
| bubble 2.5 secs | | | | | | | | | | |
| Concentration pass | 34 | 20.3 | 0 | 0 | 7 | 15 | 15 | 69.7 | 83.0 | 4 |
| Concentration pass | 35 | 0 | 3 | 7 | 0 | 15 | 15 | 83.0 | 87.0 | 4 |
| Concentration pass | 36 | 3 | 0 | 0 | 1 | 15 | 15 | 87.0 | 89.0 | 4 |
| Concentration pass | 37 | 0 | 0.4 | 1 | 0 | 15 | 15 | 89.0 | 89.6 | 4 |
| Concentration pass | 38 | 0.4 | 0 | 0 | 0.3 | 15 | 15 | 89.6 | 89.7 | 4 |
| Elution from | 39 | | | | | 15 | 15 | 89.7 | 89.7 | 4 |
| permeate side | | | | | | | | | | |
| Sweep to RR1 | 40 | | 0.5 | | | 15 | 15 | 89.7 | 89.7 | 4 |
| Open manifold and | 41 | 0.5 | 0 | 0 | 0 | 15 | 15 | 89.7 | 89.7 | 4 |
| recover culture | | | | | | | | | | |
| from RR1 | | | | | | | | | | |

TABLE 3
Protocol System State E. coli

| | | | | | | | | | | | RR1 | RR1 | RR2 | RR2 | |
|---|---|---|---|---|---|---|---|---|---|---|---|---|---|---|---|
| | Manifold/ | | | | | | | | FM1 | FM2 | Initial | Final | Initial | Final | |
| Description of step | arm | pump | SV1 | SV2 | SV3 | SV4 | P1 | P2 | Spike? | Spike? | vol | vol | vol | vol | Temp |
| Load TFF cartridge on instrument | O | 0 | 0 | 0 | 0 | 0 | 0 | 0 | NA | NA | 0 | 0 | 0 | 0 | RT |
| Transfer 20 mL cell culture to RR1 | O | 0 | 0 | 0 | 0 | 0 | 0 | 0 | NA | NA | 0 | 20 | 0 | 0 | 4 |
| Close manifold | C | 0 | 0 | 0 | 0 | 0 | 0 | 0 | NA | NA | 20 | 20 | 0 | 0 | 4 |
| Concentration pass 1.1 | C | 1 | 1 | 0 | 0 | 0 | 24 | 24 | NO | YES | 20 | 0 | 0 | 12.2 | 4 |
| Concentration pass 1.2 | C | 1 | 0 | 1 | 0 | 0 | 24 | 24 | YES | NO | 0 | 8 | 12.2 | 0 | 4 |
| Concentration pass 1.3 | C | 1 | 1 | 0 | 0 | 0 | 24 | 24 | NO | YES | 8 | 0 | 0 | 5.5 | 4 |
| Concentration pass 1.4 | C | 1 | 0 | 1 | 0 | 0 | 15 | 15 | YES | NO | 0 | 3.6 | 5.5 | 0 | 4 |
| Concentration pass 1.5 | C | 1 | 1 | 0 | 0 | 0 | 15 | 15 | NO | YES | 3.6 | 0 | 0 | 2.6 | 4 |
| [Continue concentration passes 1.6, | C | 1 | 1 | 1 | 0 | 0 | 6 | 7 | YES | NO | 0 | 0 | 2.6 | 0 | 4 |
| 1.8, 1.10 . . . until time <2] | | | | | | | | | | | | | | | |
| [Continue concentration passes 1.7, | C | 1 | 1 | 1 | 0 | 0 | 7 | 6 | NO | YES | TBD | 0 | 0 | 0 | 4 |
| 1.9, 1.11 . . . until time <2] | | | | | | | | | | | | | | | |
| Open manifold and transfer 3 mL | O | 0 | 0 | 0 | 0 | 0 | 0 | 0 | NA | NA | 0 | 3 | 0 | 0 | 4 |
| buffer to RR1 | | | | | | | | | | | | | | | |
| Open manifold and transfer 15 mL | O | 0 | 0 | 0 | 0 | 0 | 0 | 0 | NA | NA | 0 | 3 | 0 | 15 | 4 |
| buffer to RR2 | | | | | | | | | | | | | | | |
| Close manifold | C | 0 | 0 | 0 | 0 | 0 | 0 | 0 | NA | NA | 3 | 3 | 15 | 15 | 4 |
| Concentrate RR1 to flush permeate | C | 1 | 1 | 0 | 0 | 0 | 24 | 0 | NO | YES | 3 | 0 | 15 | 17.1 | 4 |
| Lift cells from permeate | C | 1 | 1 | 1 | 1 | 1 | −7 | −7 | NO | NO | 0 | 0.1 | 17.1 | 17.2 | 4 |
| Transfer cells to RR1 | C | 1 | 1 | 0 | 1 | 1 | −7 | 0 | NO | YES | 0 | 17.3 | 17.2 | 0 | |
| Concentration pass 2.1 | C | 1 | 1 | 0 | 0 | 0 | 24 | 24 | NO | YES | 17.3 | 0 | 0 | 12.1 | 4 |
| Concentration pass 2.2 | C | 1 | 0 | 1 | 0 | 0 | 24 | 24 | YES | NO | 0 | 8.5 | 12.1 | 0 | 4 |
| Concentration pass 2.3 | C | 1 | 1 | 0 | 0 | 0 | 24 | 24 | NO | YES | 8.5 | 0 | 0 | 5.9 | 4 |
| Concentration pass 2.4 | C | 1 | 0 | 1 | 0 | 0 | 15 | 15 | YES | NO | 0 | 4.2 | 5.9 | 0 | 4 |
| Concentration pass 2.5 | C | 1 | 1 | 0 | 0 | 0 | 15 | 15 | NO | YES | 4.2 | 0 | 0 | 2.9 | 4 |
| [Continue concentration passes 2.6, | C | 1 | 1 | 1 | 0 | 0 | 6 | 7 | YES | NO | 0 | TBD | 2.9 | 0 | 4 |
| 2.8, 2.10 . . . until time <2] | | | | | | | | | | | | | | | |
| [Continue concentration passes 2.7, | C | 1 | 1 | 1 | 0 | 0 | 7 | 6 | NO | YES | TBD | 0 | 0 | 0 | 4 |
| 2.9, 2.11 . . . until time <2] | | | | | | | | | | | | | | | |
| Open manifold and transfer 3 mL | O | 0 | 0 | 0 | 0 | 0 | 0 | 0 | NA | NA | 0 | 3 | 0 | 0 | 4 |
| buffer to RR1 | | | | | | | | | | | | | | | |
| Open manifold and transfer 15 mL | O | 0 | 0 | 0 | 0 | 0 | 0 | 0 | NA | NA | 3 | 3 | 0 | 15 | 4 |
| buffer to RR2 | | | | | | | | | | | | | | | |
| Close manifold | C | 0 | 0 | 0 | 0 | 0 | 0 | 0 | NA | NA | 3 | 3 | 15 | 15 | 4 |
| Concentrate RR1 to flush permeate | C | 1 | 1 | 0 | 0 | 0 | 24 | 0 | NO | YES | 3 | 0 | 15 | 17.1 | 4 |
| Lift cells from permeate | C | 1 | 1 | 1 | 1 | 1 | −7 | −7 | NO | NO | 0 | 0.1 | 17.1 | 17.2 | 4 |
| Transfer cells to RR1 | | 1 | 1 | 0 | 1 | 1 | −7 | 0 | NO | YES | 0 | 17.3 | 17.2 | 0 | |
| Concentration pass 3.1 | C | 1 | 1 | 0 | 0 | 0 | 24 | 24 | NO | YES | 17.3 | 0 | 0 | 12.1 | 4 |
| Concentration pass 3.2 | C | 1 | 0 | 1 | 0 | 0 | 24 | 24 | YES | NO | 0 | 8.5 | 12.1 | 0 | 4 |
| Concentration pass 3.3 | C | 1 | 1 | 0 | 0 | 0 | 24 | 24 | NO | YES | 8.5 | 0 | 0 | 5.9 | 4 |
| Concentration pass 3.4 | C | 1 | 0 | 1 | 0 | 0 | 15 | 15 | YES | NO | 0 | 4.2 | 5.9 | 0 | 4 |
| Concentration pass 3.5 | C | 1 | 1 | 0 | 0 | 0 | 15 | 15 | NO | YES | 4.2 | 0 | 0 | 2.9 | 4 |
| [Continue concentration passes 3.6, | C | 1 | 1 | 1 | 0 | 0 | 6 | 7 | YES | NO | 0 | TBD | 2.9 | 0 | 4 |
| 3.8, 3.10 . . . until time <2] | | | | | | | | | | | | | | | |
| [Continue concentration passes 3.7, | C | 1 | 1 | 1 | 0 | 0 | 7 | 6 | NO | YES | TBD | 0 | 0 | 0 | 4 |
| 3.9, 3.11 . . . until time <2] | | | | | | | | | | | | | | | |
| Open manifold and transfer 3 mL | O | 0 | 0 | 0 | 0 | 0 | 0 | 0 | NA | NA | 0 | 3 | 0 | 0 | 4 |
| buffer to RR1 | | | | | | | | | | | | | | | |
| Open manifold and transfer 15 mL | O | 0 | 0 | 0 | 0 | 0 | 0 | 0 | NA | NA | 3 | 3 | 0 | 15 | 4 |
| buffer to RR2 | | | | | | | | | | | | | | | |
| Close manifold | C | 0 | 0 | 0 | 0 | 0 | 0 | 0 | NA | NA | 3 | 3 | 15 | 15 | 4 |
| Concentrate RR1 to flush permeate | C | 1 | 1 | 0 | 0 | 0 | 24 | 0 | NO | YES | 3 | 0 | 15 | 17.1 | 4 |
| Lift cells from permeate | C | 1 | 1 | 1 | 1 | 1 | −7 | −7 | NO | NO | 0 | 0.1 | 17.1 | 17.2 | 4 |
| Transfer cells to RR1 | C | 1 | 1 | 0 | 1 | 1 | −7 | 0 | NO | YES | 0 | 17.3 | 17.2 | 0 | 4 |
| Concentration pass E1 | C | 1 | 1 | 0 | 0 | 0 | 24 | 0 | NO | YES | 17.3 | 0 | 0 | 12.3 | 4 |
| Lift cells from permeate | C | 1 | 1 | 1 | 1 | 1 | −7 | −7 | NO | NO | 0 | 0.1 | 12.3 | 12.2 | 4 |
| Transfer cells to RR2 | C | 1 | 0 | 1 | 1 | 1 | 0 | −7 | YES | NO | 0 | 0 | 12.2 | 12.1 | 4 |
| Concentration pass E2 | C | 1 | 0 | 1 | 0 | 0 | 0 | 24 | YES | NO | 0 | 8.5 | 12.1 | 0 | 4 |
| Lift cells from permeate | C | 1 | 1 | 1 | 1 | 1 | −7 | −7 | NO | NO | 8.5 | 8.6 | 0 | 0.1 | 4 |
| Transfer cells to RR1 | C | 1 | 1 | 0 | 1 | 1 | −7 | 0 | NO | YES | 8.6 | 8.7 | 0.1 | 0 | 4 |
| Concentration pass E3 | C | 1 | 1 | 0 | 0 | 0 | 24 | 0 | NO | YES | 8.7 | 0 | 0 | 6.1 | 4 |
| Lift cells from permeate | C | 1 | 1 | 1 | 1 | 1 | −7 | −7 | NO | NO | 0 | 0.1 | 6.1 | 6.2 | 4 |
| Transfer cells to RR2 | C | 1 | 0 | 1 | 1 | 1 | 0 | −7 | YES | NO | 0 | 0 | 6.2 | 6.3 | 4 |
| Concentration pass E4 | C | 1 | 0 | 1 | 0 | 0 | 0 | 15 | YES | NO | 0 | 4.3 | 6.3 | 0 | 4 |
| Lift cells from permeate | C | 1 | 1 | 1 | 1 | 1 | −7 | −7 | NO | NO | 4.3 | 4.4 | 0 | 0.1 | 4 |
| Transfer cells to RR1 | C | 1 | 1 | 0 | 1 | 1 | −7 | 0 | NO | YES | 4.4 | 4.5 | 0.1 | 0 | 4 |
| Concentration pass E5 | C | 1 | 1 | 0 | 0 | 0 | 15 | 0 | NO | YES | 4.5 | 0 | 0 | 3.1 | 4 |
| Lift cells from permeate | C | 1 | 1 | 1 | 1 | 1 | −7 | −7 | NO | NO | 0 | 0.1 | 3.1 | 3.2 | 4 |
| Transfer cells to RR2 | C | 1 | 0 | 1 | 1 | 1 | 0 | −7 | YES | NO | 0 | 0 | 3.2 | 3.2 | 4 |
| [Continue concentration passes | C | 1 | 1 | 1 | 0 | 0 | 6 | 7 | NA | NA | 0 | TBD | 3.3 | 0 | 4 |
| until volume <3] | | | | | | | | | | | | | | | |
| Lift cells from permeate | C | 1 | 1 | 1 | 1 | 1 | −7 | −7 | NO | NO | TBD | TBD | 0 | 0.1 | 4 |
| Transfer cells to RR1 | C | 1 | 1 | 0 | 1 | 1 | −7 | 0 | YES | NO | TBD | TBD | 0.1 | 0 | 4 |
| [Continue concentration passes | C | 1 | 1 | 1 | 0 | 0 | 7 | 6 | NO | YES | TBD | 0 | 0 | 0 | 4 |
| until volume <3] | | | | | | | | | | | | | | | |
| Lift cells from permeate | C | 1 | 1 | 1 | 1 | 1 | −7 | −7 | NO | NO | 0 | 0.1 | 0 | 0.1 | 4 |
| Transfer cells to RR2 | C | 1 | 0 | 1 | 1 | 1 | 0 | −7 | YES | NO | 0 | 0 | 0.1 | 0.2 | 4 |
| Lift cells from permeate and pull | O | 0 | 0 | 0 | 0 | 0 | 0 | 0 | NA | NA | 0.5 | 0 | 0 | 0 | 4 |
| cell suspension one final time | | | | | | | | | | | | | | | |

TABLE 4
SWIIN Design Valve Status and Prop Valve PSI

| | | | | | | | | | | | Prop | Prop |
|---|---|---|---|---|---|---|---|---|---|---|---|---|
| | | Manifold | Manifold | | SV- | SV- | SV1 | SV2 | SV3 | SV4 | Valve 1 | Valve 2 |
| Description of step | Step | ARM 1 | ARM 2 | Pump | POS | NEG | (RR1) | (PR1) | (PR2) | (RR2) | (psi) | (psi) |
| Load SWIIN cartridge | 1 | open | open | 0 | 0 | 0 | 0 | 0 | 0 | 0 | 0 | 0 |
| on the instrument | | | | | | | | | | | | |
| Transfer 10 mL PBS- | 2 | open | closed | 0 | 0 | 0 | 0 | 0 | 0 | 0 | 0 | 0 |
| 0.01% Tween80 from | | | | | | | | | | | | |
| Reagent Strip to PR1 | | | | | | | | | | | | |
| Load PBS- | 3 | closed | sealed | 1 | 1 | 0 | 0 | 1 | 0 | 0 | 0.5 | 0.5 |
| 0.01% Tween80 into | | | | | | | | | | | | |
| Permeate channel - | | | | | | | | | | | | |
| Bubble Flush | | | | | | | | | | | | |
| Load PBS- | 4 | closed | sealed | 1 | 1 | 0 | 1 | 1 | 0 | 1 | 0.5 | 0.5 |
| 0.01% Tween80 into | | | | | | | | | | | | |
| Permeate channel - | | | | | | | | | | | | |
| Fill Channel | | | | | | | | | | | | |
| Flood Retentate - | 5 | sealed | sealed | 1 | 0 | 1 | 1 | 0 | 0 | 1 | −0.7 | −0.7 |
| Symmetrically Apply | | | | | | | | | | | | |
| Vacuum to Retentate | | | | | | | | | | | | |
| Flood Retentate - | 6 | sealed | sealed | 1 | 0 | 1 | 0 | 0 | 0 | 1 | 0 | −0.7 |
| Sweep to RR2 | | | | | | | | | | | | |
| Aspirate liquid out of | 7 | open | open | 1 | 0 | 0 | 0 | 0 | 0 | 0 | 0 | 0 |
| RR1 & RR2 | | | | | | | | | | | | |
| Transfer 9.5 mL of | 8 | open | closed | 1 | 0 | 0 | 0 | 0 | 0 | 0 | 0 | 0 |
| PBS-0.01% Tween80 | | | | | | | | | | | | |
| from Reagent Strip | | | | | | | | | | | | |
| to RR1 | | | | | | | | | | | | |
| Transfer 0.5 mL cell | 9 | open | closed | 1 | 0 | 0 | 0 | 0 | 0 | 0 | 0 | 0 |
| Solution from FTEP | | | | | | | | | | | | |
| to RR1 | | | | | | | | | | | | |
| Pipette cell solution | 10 | open | closed | 1 | 0 | 0 | 0 | 0 | 0 | 0 | 0 | 0 |
| up/down in RR1 | | | | | | | | | | | | |
| Pull cell solution | 11 | open | sealed | 1 | 0 | 1 | 0 | 0 | 0 | 1 | 0 | −0.7 |
| from RR1 into | | | | | | | | | | | | |
| Retentate Channel | | | | | | | | | | | | |
| Pull retentate | 12 | sealed | sealed | 1 | 0 | 1 | 0 | 1 | 1 | 0 | −0.7 | −0.7 |
| through membrane | | | | | | | | | | | | |
| (low vac) | | | | | | | | | | | | |
| Pull retentate | 13 | sealed | sealed | 1 | 0 | 1 | 0 | 1 | 1 | 0 | −1 | −1 |
| through membrane | | | | | | | | | | | | |
| (high vac) | | | | | | | | | | | | |
| Sweep all fluid to PR1 | 14 | sealed | sealed | 1 | 1 | 0 | 1 | 0 | 1 | 1 | 0.5 | .05 |
| Aspirate liquid out of | 15 | open | open | 1 | 0 | 0 | 0 | 0 | 0 | 0 | 0 | 0 |
| PR1 & PR2 | | | | | | | | | | | | |
| Transfer 10 mL media | 16 | open | closed | 1 | 0 | 0 | 0 | 0 | 0 | 0 | 0 | 0 |
| from Reagent Strip to | | | | | | | | | | | | |
| PR1 | | | | | | | | | | | | |
| Load media from PR1 | 17 | open | closed | 1 | 0 | 1 | 0 | 0 | 1 | 0 | 0 | −0.7 |
| into Permeate | | | | | | | | | | | | |
| channel | | | | | | | | | | | | |
| Aspirate liquid out of | 18 | open | open | 0 | 0 | 0 | 0 | 0 | 0 | 0 | 0 | 0 |
| PR1 & PR2 | | | | | | | | | | | | |
| INCUBATE SWIIN 30 C. | 19 | closed | closed | 0 | 0 | 0 | 0 | 0 | 0 | 0 | 0 | 0 |
| #1 - may require | | | | | | | | | | | | |
| intermittent airflow, | | | | | | | | | | | | |
| media rinses | | | | | | | | | | | | |
| Ramp up (30 C. to | 20 | closed | closed | 0 | 0 | 0 | 0 | 0 | 0 | 0 | 0 | 0 |
| 42 C.) | | | | | | | | | | | | |
| INCUBATE SWIIN | 21 | closed | closed | 0 | 0 | 0 | 0 | 0 | 0 | 0 | 0 | 0 |
| 42 C. - may require | | | | | | | | | | | | |
| intermittent airflow, | | | | | | | | | | | | |
| media rinses | | | | | | | | | | | | |
| Ramp down (42 C. to | 22 | closed | closed | 0 | 0 | 0 | 0 | 0 | 0 | 0 | 0 | 0 |
| 30 C.) | | | | | | | | | | | | |
| INCUBATE SWIIN 30 C. | 23 | closed | closed | 0 | 0 | 0 | 0 | 0 | 0 | 0 | 0 | 0 |
| #2 - may require | | | | | | | | | | | | |
| intermittent airflow, | | | | | | | | | | | | |
| media rinses | | | | | | | | | | | | |
| Pull media out of | 24 | sealed | sealed | 1 | 0 | 1 | 0 | 0 | 1 | 0 | 0 | −0.7 |
| Permeate channel | | | | | | | | | | | | |
| into PR2 | | | | | | | | | | | | |
| Aspirate liquid out of | 25 | open | open | 1 | 0 | 0 | 0 | 0 | 0 | 0 | 0 | 0 |
| PR2 | | | | | | | | | | | | |
| Transfer 10 mL | 26 | open | closed | 1 | 0 | 0 | 0 | 0 | 0 | 0 | 0 | 0 |
| media + 10% glycerol | | | | | | | | | | | | |
| from Reagent Strip to | | | | | | | | | | | | |
| PR1 | | | | | | | | | | | | |
| Pull | 27 | open | sealed | 1 | 0 | 1 | 0 | 0 | 1 | 0 | 0 | −0.7 |
| media + 10% glycerol | | | | | | | | | | | | |
| from PR1 into | | | | | | | | | | | | |
| Permeate channel | | | | | | | | | | | | |
| Flood Retentate - | 28 | sealed | sealed | 1 | 1 | 0 | 0 | 1 | 1 | 0 | 1 | 1 |
| Dislodge Cells | | | | | | | | | | | | |
| Sweep all fluid to RR2 | 29 | sealed | sealed | 1 | 0 | 1 | 0 | 0 | 0 | 1 | 0 | −0.7 |
| Aspirate 5 mL cell | 30 | closed | open | 0 | 0 | 0 | 0 | 0 | 0 | 0 | 0 | 0 |
| solution from RR2 | | | | | | | | | | | | |
| into final vial | | | | | | | | | | | | |
| Aspirate liquid out of | 31 | open | open | 0 | 0 | 0 | 0 | 0 | 0 | 0 | 0 | 0 |
| RR1 & RR2 | | | | | | | | | | | | |
| Unload SWIIN | 32 | open | open | 0 | 0 | 0 | 0 | 0 | 0 | 0 | 0 | 0 |

TABLE 5
SWIIN Design Flow Meter Status

| | FM1 (PR2) | FM1 (PR2) | FM2 (RR2) | FM2 (RR2) | Valve delay | Requires | |
|---|---|---|---|---|---|---|---|
| Description of step | Detection | Threshold | Detection | Threshold | after spike(s) | pLLD | Duration (sec) |
| Load SWIIN cartridge on the | NA | NA | NA | NA | NA | 0 | N/A |
| instrument | | | | | | | |
| Transfer 10 mL PBS- | NA | NA | NA | NA | NA | 0 | as needed |
| 0.01% Tween80 from | | | | | | | |
| Reagent Strip to PR1 | | | | | | | |
| Load PBS-0.01% Tween80 into | NA | NA | NA | NA | NA | 0 | 0.5 |
| Permeate channel - Bubble Flush | | | | | | | |
| Load PBS-0.01% Tween80 into | NA | NA | FALL | 10 | 5 | 0 | until FM trigger |
| Permeate channel - Fill Channel | | | | | | | |
| Flood Retentate - Symmetrically Apply | NA | NA | NA | NA | NA | 0 | 30 |
| Vacuum to Retentate | | | | | | | |
| Flood Retentate - Sweep to RR2 | NA | NA | NA | NA | NA | 0 | 60 |
| Aspirate liquid out of RR1 & RR2 | NA | NA | NA | NA | NA | 0 | determined by |
| | | | | | | | ADP |
| Transfer 9.5 mL of PBS-0.01% Tween80 | NA | NA | NA | NA | NA | 0 | determined by |
| from Reagent Strip to RR1 | | | | | | | ADP |
| Transfer 0.5 mL cell Solution from FTEP | NA | NA | NA | NA | NA | 0 | determined by |
| to RR1 | | | | | | | ADP |
| Pipette cell solution up/down in RR1 | NA | NA | NA | NA | NA | 0 | 10 |
| Pull cell solution from RR1 into | NA | NA | NA | NA | NA | 1 | until RR1 & RR2 |
| Retentate Channel | | | | | | | are equal volume |
| Pull retentate through membrane | NA | NA | NA | NA | NA | 0 | 90 |
| (low vac) | | | | | | | |
| Pull retentate through membrane | NA | NA | NA | NA | NA | 0 | 30 |
| (high vac) | | | | | | | |
| Sweep all fluid to PR1 | RISE | 50 | NA | NA | 0 | 0 | until FM trigger |
| Aspirate liquid out of PR1 & PR2 | NA | NA | NA | NA | NA | 0 | determined by |
| | | | | | | | ADP |
| Transfer 10 mL media from Reagent | NA | NA | NA | NA | NA | 0 | determined by |
| Strip to PR1 | | | | | | | ADP |
| Load media from PR1 into Permeate | NA | NA | NA | NA | NA | 1 | until PR1 is |
| channel | | | | | | | nearly exhausted |
| Aspirate liquid out of PR1 & PR2 | NA | NA | NA | NA | NA | 0 | determined by |
| | | | | | | | ADP |
| INCUBATE SWIIN 30 C. #1 - may require | NA | NA | NA | NA | NA | 0 | 32400 |
| intermittent airflow, media rinses | | | | | | | |
| Ramp up (30 C. to 42 C.) | NA | NA | NA | NA | NA | 0 | 900 |
| INCUBATE SWIIN 42 C. - may require | NA | NA | NA | NA | NA | 0 | 7200 |
| intermittent airflow, media rinses | | | | | | | |
| Ramp down (42 C. to 30 C.) | NA | NA | NA | NA | NA | 0 | 900 |
| INCUBATE SWIIN 30 C. #2 - may require | NA | NA | NA | NA | NA | 0 | 32400 |
| intermittent airflow, media rinses | | | | | | | |
| Pull media out of Permeate channel | RISE | 50 | NA | NA | 0 | 0 | until FM trigger |
| into PR2 | | | | | | | |
| Aspirate liquid out of PR2 | NA | NA | NA | NA | NA | 0 | determined by |
| | | | | | | | ADP |
| Transfer 10 mL media + 10% glycerol | NA | NA | NA | NA | NA | 0 | determined by |
| from Reagent Strip to PR1 | | | | | | | ADP |
| Pull media + 10% glycerol from PR1 into | NA | NA | NA | NA | NA | 1 | until PR1 and |
| Permeate channel | | | | | | | PR2 are equal |
| | | | | | | | volume |
| Flood Retentate - Dislodge Cells | NA | NA | NA | NA | NA | 0 | 30 |
| Sweep all fluid to RR2 | NA | NA | RISE | 50 | 0 | 0 | until FM trigger |
| Aspirate 5 mL cell solution from RR2 | NA | NA | NA | NA | NA | 0 | determined by |
| into final vial | | | | | | | ADP |
| Aspirate liquid out of RR1 & RR2 | NA | NA | NA | NA | NA | 0 | determined by |
| | | | | | | | ADP |
| Unload SWIIN | NA | NA | NA | NA | NA | 0 | N/A |

TABLE 6
SWIIN Design Reservoir Volumes
Volumes (Assume: SWIIN Volume = 5 mL)

| | RR1 | RR1 | PR1 | PR1 | PR2 | PR2 | RR2 | RR2 | Temperature | |
|---|---|---|---|---|---|---|---|---|---|---|
| Description of step | Initial | Final | Initial 2 | Final 2 | Initial 3 | Final 3 | Initial 4 | Final 4 | (° C.) | Notes |
| Load SWIIN cartridge | 0.0 | 0.0 | 0.0 | 0.0 | 0.0 | 0.0 | 0.0 | 0.0 | Continue Ramp Up | |
| on the instrument | | | | | | | | | (RT to 30) | |
| Transfer 10 mL PBS- | 0.0 | 0.0 | 10.0 | 10.0 | 0.0 | 0.0 | 0.0 | 0.0 | Continue Ramp Up | |
| 0.01% Tween80 from | | | | | | | | | (RT to 30) | |
| Reagent Strip to PR1 | | | | | | | | | | |
| Load PBS- | 0.0 | 0.0 | 10.0 | 9.8 | 0.0 | 0.0 | 0.0 | 0.0 | Continue Ramp Up | This 0.5 s step consumes |
| 0.01% Tween80 into | | | | | | | | | (RT to 30) | very little liquid |
| Permeate channel - | | | | | | | | | | |
| Bubble Flush | | | | | | | | | | |
| Load PBS- | 0.0 | 0.0 | 9.8 | 2.5 | 0.0 | 2.5 | 0.0 | 0.0 | Continue Ramp Up | Requires debounce |
| 0.01% Tween80 into | | | | | | | | | (RT to 30) | delay for flow sensor to |
| Permeate channel - | | | | | | | | | | reach high, trigger |
| Fill Channel | | | | | | | | | | threshold untested |
| Flood Retentate - | 0.0 | 2.5 | 2.5 | 0.0 | 2.5 | 0.0 | 0.0 | 2.5 | Continue Ramp Up | |
| Symmetrically Apply | | | | | | | | | (RT to 30) | |
| Vacuum to Retentate | | | | | | | | | | |
| Flood Retentate - | 2.5 | 0.0 | 0.0 | 0.0 | 0.0 | 0.0 | 2.5 | 10.0 | Continue Ramp Up | |
| Sweep to RR2 | | | | | | | | | (RT to 30) | |
| Aspirate liquid out of | 0.0 | 0.0 | 0.0 | 0.0 | 0.0 | 0.0 | 10.0 | 0.0 | Continue Ramp Up | |
| RR1 & RR2 | | | | | | | | | (RT to 30) | |
| Transfer 9.5 mL of | 0.0 | 9.5 | 0.0 | 0.0 | 0.0 | 0.0 | 0.0 | 0.0 | Continue Ramp Up | |
| PBS-0.01% Tween80 | | | | | | | | | (RT to 30) | |
| from Reagent Strip | | | | | | | | | | |
| to RR1 | | | | | | | | | | |
| Transfer 0.5 mL cell | 0.0 | 10.0 | 0.0 | 0.0 | 0.0 | 0.0 | 0.0 | 0.0 | Continue Ramp Up | |
| Solution from FTEP | | | | | | | | | (RT to 30) | |
| to RR1 | | | | | | | | | | |
| Pipette cell solution | 0.0 | 10.0 | 0.0 | 0.0 | 0.0 | 0.0 | 0.0 | 0.0 | Continue Ramp Up | |
| up/down in RR1 | | | | | | | | | (RT to 30) | |
| Pull cell solution | 10.0 | 2.5 | 0.0 | 0.0 | 0.0 | 0.0 | 0.0 | 2.5 | Continue Ramp Up | |
| from RR1 into | | | | | | | | | (RT to 30) | |
| Retentate Channel | | | | | | | | | | |
| Pull retentate | 2.5 | 0.0 | 0.0 | 2.5 | 0.0 | 2.5 | 2.5 | 0.0 | Continue Ramp Up | |
| through membrane | | | | | | | | | (RT to 30) | |
| (low vac) | | | | | | | | | | |
| Pull retentate | 0.0 | 0.0 | 2.5 | 2.5 | 2.5 | 2.5 | 0.0 | 0.0 | Continue Ramp Up | This step is only here as a |
| through membrane | | | | | | | | | (RT to 30) | safeguard, all liquid |
| (high vac) | | | | | | | | | | should have transferred |
| | | | | | | | | | | in prev step |
| Sweep all fluid to PR1 | 0.0 | 0.0 | 2.5 | 10.0 | 2.5 | 0.0 | 0.0 | 0.0 | Continue Ramp Up | |
| | | | | | | | | | (RT to 30) | |
| Aspirate liquid out of | 0.0 | 0.0 | 10.0 | 0.0 | 0.0 | 0.0 | 0.0 | 0.0 | Continue Ramp Up | |
| PR1 & PR2 | | | | | | | | | (RT to 30) | |
| Transfer 10 mL media | 0.0 | 0.0 | 0.0 | 10.0 | 0.0 | 0.0 | 0.0 | 0.0 | Continue Ramp Up | |
| from Reagent Strip to | | | | | | | | | (RT to 30) | |
| PR1 | | | | | | | | | | |
| Load media from PR1 | 0.0 | 0.0 | 10.0 | 0.5 | 0.0 | 4.5 | 0.0 | 0.0 | Continue Ramp Up | |
| into Permeate channel | | | | | | | | | (RT to 30) | |
| Aspirate liquid out of | 0.0 | 0.0 | 0.5 | 0.0 | 4.5 | 0.0 | 0.0 | 0.0 | Continue Ramp Up | May keep some media in |
| PR1 & PR2 | | | | | | | | | (RT to 30) | PR1/PR2 reservoirs |
| | | | | | | | | | | during incubation |
| INCUBATE SWIIN 30 C. | 0.0 | 0.0 | 0.0 | 0.0 | 0.0 | 0.0 | 0.0 | 0.0 | 30 | 9 hours; may |
| #1 - may require | | | | | | | | | | intermittently seal |
| intermittent airflow, | | | | | | | | | | manifold arms for |
| media rinses | | | | | | | | | | airflow, media rinses |
| Ramp up (30 C. to | 0.0 | 0.0 | 0.0 | 0.0 | 0.0 | 0.0 | 0.0 | 0.0 | Ramp Up | 15 minutes; Ramp rate |
| 42 C.) | | | | | | | | | (30 to 42) | still being worked by G8 |
| INCUBATE SWIIN | 0.0. | 0.0 | 0.0 | 0.0 | 0.0 | 0.0 | 0.0 | 0.0 | 42 | 2 hours; may |
| 42 C. - may require | | | | | | | | | | intermittently seal |
| intermittent airflow, | | | | | | | | | | manifold arms for |
| media rinses | | | | | | | | | | airflow, media rinses |
| Ramp down (42 C. to | 0.0 | 0.0 | 0.0 | 0.0 | 0.0 | 0.0 | 0.0 | 0.0 | Ramp Down | 15 minutes; Ramp rate |
| 30 C.) | | | | | | | | | (42 to 30) | still being worked on by G8 |
| INCUBATE SWIIN 30 C. | 0.0 | 0.0 | 0.0 | 0.0 | 0.0 | 0.0 | 0.0 | 0.0 | 30 | 9 hours; may |
| #2 - may require | | | | | | | | | | intermittently seal |
| intermittent airflow, | | | | | | | | | | manifold arms for |
| media rinses | | | | | | | | | | airflow, media rinses |
| Pull media out of | 0.0 | 0.0 | 0.0 | 0.0 | 0.0 | 10.0 | 0.0 | 0.0 | Ramp Down | |
| Permeate channel | | | | | | | | | (30 to RT) | |
| into PR2 | | | | | | | | | | |
| Aspirate liquid out of | 0.0 | 0.0 | 0.0 | 0.0 | 0.0 | 0.0 | 0.0 | 0.0 | Continue Ramp | |
| PR2 | | | | | | | | | Down (30 to RT) | |
| Transfer 10 mL | 0.0 | 0.0 | 0.0 | 10.0 | 0.0 | 0.0 | 0.0 | 0.0 | Continue Ramp | |
| media + 10% glycerol | | | | | | | | | Down (30 to RT) | |
| from Reagent Strip to | | | | | | | | | | |
| PR1 | | | | | | | | | | |
| Pull | 0.0 | 0.0 | 10.0 | 2.5 | 0.0 | 2.5 | 0.0 | 0.0 | Continue Ramp | |
| media + 10% glycerol | | | | | | | | | Down (30 to RT) | |
| from PR1 into | | | | | | | | | | |
| Permeate channel | | | | | | | | | | |
| Flood Retentate - | 0.0 | 2.5 | 2.5 | 0.0 | 2.5 | 0.0 | 0.0 | 2.5 | Continue Ramp | |
| Dislodge Cells | | | | | | | | | Down (30 to RT) | |
| Sweep all fluid to RR2 | 2.5 | 0.0 | 0.0 | 0.0 | 0.0 | 0.0 | 0.0 | 10.0 | Continue Ramp | |
| | | | | | | | | | Down (30 to RT) | |
| Aspirate 5 mL cell | 0.0 | 0.0 | 0.0 | 0.0 | 0.0 | 0.0 | 0.0 | 5.0 | Continue Ramp | |
| solution from RR2 | | | | | | | | | Down (30 to RT) | |
| into final vial | | | | | | | | | | |
| Aspirate liquid out of | 0.0 | 0.0 | 0.0 | 0.0 | 0.0 | 0.0 | 0.0 | 0.0 | Continue Ramp | |
| RR1 & RR2 | | | | | | | | | Down (30 to RT) | |
| Unload SWIIN | 0.0 | 0.0 | 0.0 | 0.0 | 0.0 | 0.0 | 0.0 | 0.0 | Continue Ramp | |
| | | | | | | | | | Down (30 to RT) | |

1. A method for concentrating a cell sample, comprising the steps of:
providing a tangential flow filtration (TFF) device comprising:
a tangential flow assembly comprising:
a retentate member comprising an upper surface and a lower surface with a retentate channel structure disposed on the lower surface of the retentate member and first and second retentate ports wherein the first retentate port is disposed at a first end of the retentate channel structure and the second retentate port is disposed at a second end of the retentate channel structure, and wherein the first and second retentate ports traverse the first member from the lower surface to the upper surface;
a permeate member comprising an upper surface and a lower surface with a permeate channel structure disposed on the upper surface of the permeate member and at least one permeate port, wherein the at least one permeate port is disposed at a first end of the permeate channel structure, wherein the at least one permeate port traverses the permeate member from the lower surface to the upper surface, and wherein the retentate and permeate channel structures mate to form a single flow channel; and
a membrane disposed between the retentate and permeate members thereby bifurcating the single flow channel into upper and lower portions;
a reservoir assembly comprising a first retentate reservoir fluidically coupled to the first retentate port, a second retentate reservoir fluidically coupled to the second retentate port and a reservoir top disposed over the first and second retentate reservoirs;
a pneumatic assembly configured to apply pressure to move liquid through the single flow channel via negative and positive pressure applied to the first and second retentate reservoirs, to monitor pressure in the retentate reservoirs, and to monitor flow in the single flow channel;
an interface between the pneumatic assembly and the reservoir top; and
means to couple the tangential flow assembly and the reservoir assembly;
providing a cell sample in a first medium;
placing the cell sample into the first retentate reservoir;
passing the cell sample from the first retentate reservoir through the retentate channel structure for a length of the single flow channel until the cell sample is transported into and retained within the second retentate reservoir;
removing filtrate through the permeate port;
passing the cell sample from the second retentate reservoir through the retentate channel structure for the length of the single flow channel until the cell sample is transported into and retained within the first retentate reservoir;
removing filtrate through the permeate port; and
repeating the passing and collecting steps until the cell sample is concentrated to a desired volume.

2. The method of claim 1, wherein the single flow channel of the TFF device has a serpentine configuration.

3. The method of claim 2, wherein the single flow channel of the TFF device has an undulating geometry.

4. The method of claim 1, wherein the single flow channel of the TFF device has an undulating geometry.

5. The method of claim 1, wherein the length of the single flow channel of the TFF device is from 50 mm to 600 mm.

6. The method of claim 5, wherein the length of the single flow channel of the TFF device is from 100 mm to 500 mm.

7. The method of claim 6, wherein the length of single flow channel of the TFF device is from 200 mm to 400 mm.

8. The method of claim 1, wherein the reservoir assembly of the TFF device further comprises a first permeate reservoir fluidically coupled to the at least one permeate port.

9. (canceled)

10. The method of claim 1, wherein the TFF device further comprises a second permeate port disposed at a second end of the permeate channel structure.

11. The method of claim 1, wherein the reservoir assembly of the TFF device further comprises a buffer reservoir fluidically coupled to at least one of the first and second retentate reservoirs.

12. The method of claim 1, wherein a cross section of the single flow channel of the TFF device is rectangular or trapezoidal.

13. The method of claim 12, wherein the cross section of the single flow channel of the TFF device is 300 μm to 700 μm wide and 300 μm to 700 μm high.

14. The method of claim 1, wherein a cross section of the single flow channel of the TFF device is generally circular.

15. The method of claim 14, wherein the cross section of the single flow channel of the TFF device is 300 μm to 700 μm in radius.

16. The method of claim 1, wherein the reservoir assembly of the TFF device further comprises a gasket disposed on the reservoir top of the reservoir assembly and wherein the gasket comprises a pneumatic port and a fluid transfer port for each of the first and second retentate reservoirs.

17. A method for concentrating a cell sample, comprising the steps of:
providing a tangential flow filtration (TFF) device comprising:
a tangential flow assembly comprising:
a retentate member comprising an upper surface and a lower surface with a retentate channel structure disposed on the lower surface of the retentate member and first and second retentate ports wherein the first retentate port is disposed at a first end of the retentate channel structure and the second retentate port is disposed at a second end of the retentate channel structure, and wherein the first and second retentate ports traverse the first member from the lower surface to the upper surface;
a permeate member comprising an upper surface and a lower surface with a permeate channel structure disposed on the upper surface of the permeate member and at least one permeate port, wherein the at least one permeate port is disposed at a first end of the permeate channel structure, wherein the at least one permeate port traverses the permeate member from the lower surface to the upper surface, and wherein the retentate and permeate channel structures mate to form a single flow channel; and
a membrane disposed between the retentate and permeate members thereby bifurcating the single flow channel into upper and lower portions;
a reservoir assembly comprising a first retentate reservoir fluidically coupled to the first retentate port, a second retentate reservoir fluidically coupled to the second retentate port, a permeate reservoir fluidically coupled to the at least one permeate port, and a reservoir top disposed over the first and second retentate reservoirs;
a pneumatic assembly configured to apply pressure to move liquid through the single flow channel via negative and positive pressure applied to the first and second retentate reservoirs, to monitor pressure in the retentate reservoirs, and to monitor flow in the single flow channel;
an interface between the pneumatic assembly and the reservoir top; and
means to couple the tangential flow assembly and the reservoir assembly;
providing a cell sample in a first medium;
placing the cell sample into the first retentate reservoir;
passing the cell sample from the first retentate reservoir through the retentate channel structure for a length of the single flow channel until the cell sample is transported into and retained within the second retentate reservoir;
removing filtrate through the permeate port;
passing the cell sample from the second retentate reservoir through the retentate channel structure for the length of the single flow channel until the cell sample is transported into and retained within the first retentate reservoir;
removing filtrate through the permeate port; and
repeating the passing and collecting steps until the cell sample is concentrated to a desired volume.

18. The method of claim 17, wherein the single flow channel of the TFF device has a serpentine configuration and an undulating geometry.

19. (canceled)

20. (canceled)

21. The method of claim 1, further comprising the steps of adding a second medium to the cells in the first and/or second reservoirs wherein the second medium is different from the first medium, and repeating the passing and collecting steps until the cell sample is suspended in the second medium.

22. The method of claim 17, further comprising the steps of adding a second medium to the cells in the first and/or second reservoirs wherein the second medium is different from the first medium, and repeating the passing and collecting steps until the cell sample is suspended in the second medium.

23. The method of claim 17, wherein the reservoir assembly of the TFF device further comprises a buffer reservoir fluidically coupled to at least one of the first and second retentate reservoirs.
